(19) United States
(12) Patent Application Publication
(10) Pub. No.: US 2010/0092470 A1
(43) Pub. Date: Apr. 15, 2010

(54) ANTIBODIES, ANALOGS AND USES THEREOF

(75) Inventors: Ram S. Bhatt, San Diego, CA (US); Rishi S. Bhatt, San Diego, CA (US); Yu Zhang, San Diego, CA (US)

Correspondence Address: FOLEY AND LARDNER LLP;SUITE 500, 3000 K STREET NW, WASHINGTON, DC, 20007, US

(73) Assignee: ICB International, Inc.

(21) Appl. No.: 12/563,330

(22) Filed: Sep. 21, 2009

Related U.S. Application Data
(60) Provisional application No. 61/192,732, filed on Sep. 22, 2008.

(51) Int. Cl. A61K 39/395; C07K 16/00; C07H 21/00; C12P 21/00; G01N 33/566; G01N 33/574; A61P 35/00

(52) U.S. Cl. 424/133.1; 530/387.3; 530/391.1; 536/23.53; 435/69.6; 436/501; 435/7.23

(57) ABSTRACT

Camelid and shark heavy chain only antibodies and their analogs are disclosed. Methods of making such antibodies and their analogs are also provided. Also provided are kits, and methods of using such antibodies and their analogs in diagnostics, prognostics, therapy, and simultaneous diagnosis and therapy.

### CROSS-REFERENCE TO RELATED APPLICATIONS

This application claims the benefit of U.S. Provisional Application 61/192,732, filed Sep. 22, 2008 which is hereby incorporated by reference in its entirety.

### FIELD OF THE INVENTION

The invention relates to camelid and shark heavy chain only antibodies, their analogs and uses thereof.

### BACKGROUND OF THE INVENTION

As early as 1983 it was suspected that the sera of camelid comprised of two different kinds of immunoglobulin: conventional heterodimeric IgGs composed of heavy and light chains, and an unconventional IgGs without the light chains [Grover Y P, et al., Indian Journal of Biochemistry and Biophysics, 20, 238 (1983)]. Grover et al. demonstrated the presence of three bands which were designated as IgM, IgA, and a broad heterogeneous band containing a mixture of IgG complexes. One can speculate that the broad band these authors observed was due to the presence of mixture of normal IgG and heavy-chain IgG without the light chain but since a proper sizing marker had not been used, coupled with the poor resolution of the bands, these authors could not satisfactorily characterize the broad IgGs band.

Ungar-Waron et al. disclosed a SDS-PAGE analysis of camelid IgGs mixture treated with and without 2-mercaptoethanol (2ME) [Israel J. Vet. Medicine, 43 (3), 198 (1987)]. In the absence of 2-ME, IgG-complex, obtained from camel serum, dissociated into two components with approximate molecular weight (MW) of 155 KDa (Conventional IgG) and 100 KDa (New IgG) on SDS-PAGE. However, in the presence of 2-ME, three bands of MW 55 KDa (gamma-like heavy chain), 22 KDa (Light chain) and an additional protein band of 43 KDa (now known as heavy-chain only camel antibody band without the light chains) were seen.

Subsequently, Azwai et al. from University of Liverpool, UK, independently confirmed the presence of an additional IgG band in camel serums with a molecular weight of 42 K Da by SDS-PAGE electrophoresis under reducing conditions [Azwai, S. M., et al., J. Comp. Path., 109, 187 (1993)].

Hamers-Casterman et al. reported similar findings, confirming independently the presence of 42 KDa IgG subclass in the sera of camels upon SDS-PAGE analysis under reducing conditions. [Nature, 363, 446 (1993) and U.S. Pat. No. 6,005,079].

Thus, two types of antibodies exist in camels, dromedaries and llamas: one a conventional hetero-tetramer having two heavy and two light chains (MW ˜150 K Da), and the other consisting of only two heavy chains, devoid of light chains (MW ˜90 to 100 K Da).

In addition to camelid antibodies having only two heavy chains and devoid of light chains, distinctly unconventional antibody isotype was identified in the serum of nurse sharks (Ginglymostoma cirratum) and wobbegong sharks (Orectolobus maculatus). The antibody was called the Ig new antigen receptors (IgNARs). They are disulfide-bonded homodimers consisting of five constant domains (CNAR) and one variable domain (VNAR). There is no light chain, and the individual variable domains are independent in solution and do not appear to associate across a hydrophobic interface (Greenberg, A. S., Avila, D., Hughes, M., Hughes, A., McKinney, E. & Flajnik, M. F. (1995) Nature 374, 168-173; Nuttall, S. D., Krishnan, U. V., Hattarki, M., De Gori, R., Irving, R. A. & Hudson P. J. (2001) Mol. Immunol. 38, 313-326, Comp. Biochem. Physiol. B., 15, 225 (1973)). There are three different types of IgNARs characterized by their time of appearance in shark development, and by their disulfide bond pattern (Diaz, M., Stanfield, R. L., Greenberg, A. S. & Flajnik, M. F. (2002) Immunogenetics 54, 501-512; Nuttall, S. D., Krishnan, U. V., Doughty, L., Pearson, K., Ryan, M. T., Hoogenraad, N. J., Hattarki, M., Carmichael, J. A., Irving, R. A. & Hudson, P. J. (2003) Eur. J. Biochem. 270, 3543-3554).

### RELEVANT REFERENCES

### Relevant Foreign and US Patents

| | |
|---|---|
| U.S. Pat. No. 7,371,849 | Methods of constructing camel antibody |
| (May, 2008) | libraries. |
| U.S. Pat. No. 6,838,254 B1 | Production of antibodies or |
| (January, 2005) | fragments thereof derived from |
| | heavy-chain immunoglobulins of |
| | camelidae. |
| U.S. Pat. No. 6,765,087 | Immunoglobulins devoid of light |
| (July, 2004) | chains. |
| U.S. Pat. No. 6,005,079 | Immunoglobulins devoid of light |
| (December, 1999) | chains. |
| U.S. Pat. No. 5,800,988 | Immunoglobulins devoid of light |
| (September, 1998) | chains. |
| WO/2002/048193 (June, 2002) | Camelidae Antibody Arrays. |
| EP 1264885 (December, 2002) | Antibody library. |
| WO/2001/090190 | Single-domain antigen-binding antibody |
| (November, 2001) | fragments derived from llama |
| | antibodies. |
| WO/2000/043507 (July, 2000) | Methods for producing antibody |
| | fragments. |
| EP 1024191 (August, 2000) | Production of chimeric antibodies from |
| | segment repertoires and displayed on |
| | phage. |
| WO/1999/042077 | Recognition molecules interacting |
| (August, 1999) | specifically with the active site or cleft |
| | of a target molecule. |

### Other References

Azwai S M et al, Serology of orthopoxvirus Camel Infection in Dromedary camels, Comp. Immun. Microbiol. Infect. Dis., 19, 65 (1996).
Kelly P J, et al, Isolation and characterization of immunoglobulin g and IgG Subclasses of the African elephant, Comp. Immun. Microbiol. Infect. Dis., 21, 65 (1998).
Linden Richard van der et al., Induction of immune responses and molecular cloning of the heavy-chain antibody repertoire of Lama glama, J. Immun. Methods, 240, 185 (2000).
Rivera H et al., Serological survey of viral antibodies in the Peruvian alpaca., Am. J. Vet. Res., 48, 189 (1987).
Kumar M et al., Biochemical studies on Indian Camels: Blood proteins and lipids, J. Sci. Industrial Res., 20c, 236 (1961).
Azwai S M et al., The isolation and characterization of camel immunoglobulin classes and subclasses, J. Comp. Path., 109, 187 (1993).
Zhang J, Tanha J. et al., Pentamerization of single-domain antibodies from phage libraries: a novel strategy for the rapid generation of high-avidity antibody reagents, J. Mol. Biol., 335, 49 (2004).
Vranken W et al., Solution structure of a llama single-domain antibody with hydrophilic residues, Biochemistry, 41, 8570 (2002).
Omidfar K et al, Production of a novel camel single-domain antibody specific for the Type iii mutant EGFR, Tumor Biology, 25, 296 (2004).
Frenken Leon G J et al., Isolation of antigen specific Llama VHH antibody fragments and their high level secretion by Saccharomyces cerevisiae, J. Biotechnology, 78, 11 (2000).
Holliger P., Hudson P, Engineered antibody fragments and the rise of single domains, Nature Biotechnology, 23, 1126 (2005).
Stanfield Robyn et al., Crystal Structure of a Shark-Single-Domain Antibody V Region in Complex with Lysozyme, Science, 305, 1770 (2004).
Lee V., et al., The Evolution of Multiple Isotype IgM Heavy Chain Genes in the Shark, J. Immunology, 180, 7461 (2008).

### SUMMARY OF THE INVENTION

In one aspect, the invention provides a polypeptide having all or a portion of at least one variable antigen-binding Vab domain of camelid and or shark heavy chain only antibody, at least ten contiguous amino acids derived from a source other than camelid and/or shark single-domain heavy chain antibodies lacking light-chains in which the polypeptide includes at least one binding site for an antigen. In one embodiment, the polypeptide includes at least two variable antigen-binding (Vab) domains of camelid and or shark heavy chain only antibody. In another embodiment, the polypeptide includes at least three, at least four or more variable (Vab) domains of camelid and or shark heavy chain only antibody. In some embodiments, the polypeptide may include domains from at least two different species such as camelid and shark, or two different camelid species such as llama, camel, alpaca and dromedaries. In some embodiments, the polypeptide may include one or more substitutions or deletions of the native amino acids.

In some embodiments, the polypeptide has composition and structures 1a-1g, 2-14, 20-24, 25-45, 50-79, 81-84, 87-91, 93-97, in which “CHX” represents at least ten contiguous amino acids derived from a source other than camelid and/or shark single-domain heavy chain antibodies lacking light-chains; “S” represents a linker; “Rn” represents all or a portion of at least one camelid or shark hinge region of single domain heavy chain antibody; L represents an entity linked to the polypeptide, and Vab represents camelid or shark variable region of single domain heavy chain antibody, “D” represents at least two amino acids comprising at least one charged amino acid, VNAR represents shark variable region of single domain heavy chain antibody, CH2 and CH3 represent constant domains 2 and 3 respectively of camelid and or shark single domain antibody lacking light chains, CH4 and CH5 represent constant domains 4 and 5 respectively of shark single domain heavy chain antibody lacking light chains.

In one embodiment, the generic composition of the polypeptide is represented by: [Vab]ₘ-S—R9 in which

Vab=Variable antigen-binding domain of camelid and/or shark single domain heavy chain antibodies;
m=1 to 10, preferably 2 to 5 such that the MW is approximately between 32 to 65 KDa for optimal biodistribution and retention in the body;
S is selected from the group consisting of groups I and II in which group I includes 1-20 amino acids of the hinge region of camelid and/or shark single domain heavy chain antibodies comprising at least one lysine and for cysteine, and group II includes hetrobifunctional linker with one end being capable of covalent binding with amino- or aldehyde group of single-domain antibodies, and the other end with an entity “R9”;
R9 represents an entity linked to Vab domain. “R9” can be detectable label, enzyme or protein (for example, horse radish peroxidase, alkaline phosphatase, luciferase, beta-galactosidase, and streptavidin), antibody, nucleic acid (for example, DNA, Modified DNA, Locked-DNA, PNA (Peptide Nucleic Acids), RNA, Si-RNA, Micro-RNA, mRNA, RNA-Conjugates/Modifications), radionucleotides (for example, Fluorine-18, Gallium-67, Krypton-81m, Rubidium-82, Technetium-99m, Indium-111, Iodine-123, Xenon-133, and Thallium-201, Yttrium-90, and Iodine-131), toxins (for example, Immunotoxins, Ricin, Saporin, Maytansinoid, and Calicheamicin), solid support (for example, Microchannels, Microfluidic Device, Micrarrays, Biosensors, Glass Slides, Glass Chambers, Magnetic Beads, and Gold Nanoparticles), and therapeutic agents (for example, nucleolytic enzymes, antibiotics, and chemotherapeutic agents such as Paclitaxel its derivatives).

In one embodiment, the generic composition of “S” is

S═X—P—Y in which X can be of NHS (N-Hydroxy-succinimide), sulfo-NHS, CHO, COOH, CN, SCN, epoxide, phosphate and other moieties capable of forming covalent bond with NH2 groups of single-domain antibodies;
Y can be maleimido, NHS, sulfo-NHS, SH, COOH, SCN, NH2, and epoxide, capable of forming a covalent bond with the thiol group of the detectable label;
P can be (CH₂CH₂O)n, wherein n=1-500; (CH₂)n1, wherein n1=1-15; (CH₂—R—NHCO)n2, wherein n2=1-100; nucleic acids; Nylon, polystyrene; polypropylene; protein; and chimeric protein-nucleic acids.

In another aspect, the invention provides a polypeptide having all or a portion of at least two variable antigen-binding (Vab) domains of camelid and or shark single domain heavy chain antibody lacking light chains, and all or a portion of at least one hinge region of camelid and or shark single domain heavy chain antibody lacking light chains, in a single polypeptide chain in which the polypeptide includes at least one binding site for an antigen. In one embodiment, the polypeptide includes at least three, at least four or more variable (Vab) domains of camelid and or shark single domain heavy chain antibody lacking light chain. In some embodiments, the polypeptide may include one or more substitutions or deletions of the native amino acids.

In one aspect, the invention provides a polypeptide comprising all or a portion of at least one variable (Vab) domain of camelid and or shark single domain heavy chain antibody lacking light chains, and all or a portion of at least one hinge region of camelid and or shark single domain heavy chain antibody. In some embodiments, the polypeptide may include one or more substitutions or deletions of the native amino acids.

In another aspect, the invention provides a composition having at least two polypeptides, in which each of the polypeptides includes all or a portion of at least one variable (Vab) domain of camelid and or shark single domain heavy chain antibody lacking light chain, all or a portion of at least one hinge region of camelid and or shark single domain heavy chain antibody lacking light chain in which at least one of the polypeptide includes at least one binding site for an antigen, and the polypeptides are linked to each other through at least one linker. The composition has improved biodistribution and retention. In one embodiment, at least one linker is a peptide bond. In another embodiment, at least one linker is other than a peptide bond. In one embodiment, the polypeptides of the composition include at least three, at least four, at least five or more variable antigen-binding (Vab) domains of camelid and or shark single domain heavy chain antibody. In some embodiments, the polypeptide may include one or more substitutions or deletions of the native amino acids.

In one embodiment of the above aspect, the composition is represented by structure 1d in which Vab represents variable antigen binding domain of camelid and or shark single domain heavy chain antibody lacking light chain, “Rn” represents all of portion of hinge region of camelid and/or shark heavy chain only antibody, “Man” represents maleic anhydride.

In one embodiment of the above aspect, the composition is represented by structures 21, 23, and 24 in which at least one of the variable antigen-binding domains is capable of binding to a biomarker associated with a disease. In the structures 21, 23, and 24, Vab represents variable antigen binding domain of camelid and/or shark single domain heavy chain antibody lacking light chain, “A” represents carbon or nitrogen atom, L represents a linker, “Rn” represents all of portion of hinge region of camelid and/or shark single domain heavy chain antibody lacking light chain.

In one embodiment of the above aspect, composition comprises four variable antigen-binding domains (Vab) of camelid and or shark single domain heavy chain antibody lacking light chain in which the structure of the composition is represented by structures 79 in which at least one of the Vab domains is capable of binding to a biomarker associated with a disease. In the structure 79, CHX may be of all or portion of CH1 region of human IgG or cysteine capable of forming s-s bonds, CHY represents CHX—R, where R represents [Vab-CHX]_{Z} and Z=1-6; S1 represents a linker such as structure 15, cysteine-s-s-cysteine; and R represents all of portion of hinge region of camelid and/or shark heavy chain only antibody, N represents at least two amino acids.

In another aspect the invention provides a polypeptide comprising all or a portion of at least two variable antigen-binding (Vab) domains of camelid and or shark single domain heavy chain antibody lacking light chain, at least ten contiguous amino acids derived from a source other than camelid and/or shark single-domain heavy chain antibodies lacking light-chains, all or a portion of at least one hinge region of camelid and or shark single domain heavy chain antibody lacking light chain in a single polypeptide chain in which at least two Vab domains bind to at least two different antigens, and the polypeptide has improved biodistribution and retention.

In one embodiment of all of the above aspects of the invention, the polypeptide includes all or a portion of at least one hinge region of camelid and or shark single domain heavy chain antibody lacking light chain. In one embodiment of all of the above aspects of the invention, the polypeptide includes all or a portion of at least one camelid and or shark single domain heavy chain constant domain 2 (CH2). In one embodiment of all of the above aspects of the invention, the polypeptide includes all or a portion of at least one camelid and or shark single domain heavy chain constant domain 3 (CH3). In one embodiment of all of the above aspects of the invention, at least one amino acid at positions 37, 44, 45, and 47 of the Vab region is selected from the group consisting of serine, glutamine, tyrosine, histidine, asparagine, threonine, aspartic acid, glutamic acid, lysine and arginine. In some embodiments, the polypeptide may include one or more substitutions or deletions of the native amino acids.

In some embodiments, the polypeptide may include domains from at least two different species such as camelid and shark, or two different camelid species such as llama, camel, alpaca and dromedaries.

In one embodiment of all of the above aspects of the invention, the polypeptide or the composition is capable of binding specifically to one or more antigens. Exemplary antigens include but not limited to AMACR; TMPRSS2-ERG; EPCA2; PSMA; PSA; HAAH; APP; ALZAS; Tau; gamma secretase; beta secretase; APO-A1; Apo-H; alfa-Synuclein; PV-1 PEDF; BDNF; Cystatin C; VGF nerve growth factor inducible; APO-E; GSK-3 binding protein; TEM1; PGD2; EGFR; EGFRT790M; Notch-4; ALDH-1; ESR-1; EGFRT790M; HER-2/neu; P53; RAS; KLKB1; SMAD4; Smad7; TNF-alfa; HPV; tPA; PCA-3; Mucin; Cadherin-2; FcRn alpha chain; cytokerratin 1-20; .Apo-H; Celuloplasmin; Apo AII; VGF; Vif; LEDGF/p75; TS101; gp120; CXCR4; CCR5; HIV protease; HIV integrase; OST-577; H1N1; CD3; CD11a; CD20; CD25; CD52; CD133; CD34; CD14; CD1-340; Protein C5; VEGF; VEGF-A; alfa-4-integrin; Glycoprotein IIb/IIIA; AP-1; IgG-E; NadD; STDs; TB; Bacillus anthracis protein; Plasmodium falciparum; cGMP directed phosphodiestrase; chain B of Clostridium botulinum neurotroxin type E protein; Borrelia VlsE protein; ACE2 receptor; SFRS4; SAMP; GPA; CD71; biomarkers for: lung cancer; bladder cancer; gastric cancer; brain cancer; breast cancer; prostate cancer; cervical cancer; colorectal cancer; oral cancer; leukemia; childhood neuroblastoma; Non-Hodgkin lymphoma; Alzheimer's disease; Parkinson's disease; AID; and protein markers for Down syndrome: TTHY, AMPB, SAMP, AIAT, AFMN, APOE, and SFRF4.

In one embodiment of all of the above aspects of the invention, the polypeptide is linked to at least one entity other than an antibody. In one embodiment, the entity can be detectable label, enzyme or protein (for example, horse radish peroxidase, alkaline phosphatase, luciferase, beta-galactosidase, and streptavidin), antibody, nucleic acid (for example, DNA, Modified DNA, Locked-DNA, PNA (Peptide Nucleic Acids), RNA, Si-RNA, Micro-RNA, mRNA, RNA-Conjugates/Modifications), radionucleotides (for example, Fluorine-18, Gallium-67, Krypton-81m, Rubidium-82, Technetium-99m, Indium-111, Iodine-123, Xenon-133, and Thallium-201, Yttrium-90, and Iodine-131), toxins (for example, Immunotoxins, Ricin, Saporin, Maytansinoid, and Calicheamicin), solid support (for example, beads, Microchannels, Microfluidic Device, Micrarrays, Biosensors, Glass Slides, Glass Chambers, Magnetic Beads, and Gold Nanoparticles), and therapeutic agents (for example, nucleolytic enzymes, antibiotics, and chemotherapeutic agents such as Paclitaxel its derivatives).

In some embodiments of the above aspects of the invention at least ten contiguous amino acids derived from a source other than camelid and/or shark single-domain heavy chain antibodies lacking light-chains can be all or portions of all or a portion constant domain 1 (CH1) of human heavy chain immunoglobulin G. In other embodiments, at least ten contiguous amino acids derived from a source other than camelid and/or shark single-domain heavy chain antibodies lacking light-chains can be CH1 domain of any immunoglobulin. In another embodiment, at least ten contiguous amino acids can be derived from peptide hormones, signal peptides, interleukins, interferon beta and gamma, growth factors. In some embodiments at least ten amino acids can be derived from the amino acid sequence of SEQ ID NO: 48-97. Exemplary peptide hormones include but not limited luteinizing hormone, follicle-stimulating hormone, prolactin, adrenocorticotrophic hormone, glucocorticoids, and growth hormone. Exemplary growth factors include but not limited to basic fibroblast growth factor, platelet derived growth factor, epidermal growth factor, pigment epithelium derived factor. Exemplary signal peptides include but not limited to peroxisomal targeting signals, nuclear localization signal.

In some embodiments, the invention provides a method for diagnosing an individual with one or more diseases. The method includes a) obtaining a sample of bodily fluid from the individual, b) detecting the presence or absence of one or more pathological biomarkers for the disease in which the detection includes utilizing the polypeptide or the composition of the above aspects of the invention such that the polypeptide or at least one of the polypeptide in the composition binds specifically to the biomarker, c) determining the level of one or more biomarkers if present in the individual's sample, d) comparing the level of one or more biomarkers to a reference values, and e) identifying the individual as having one or more diseases when the level of one or more biomarkers in the individual's sample is higher than the reference values. In some embodiments, the reference values are the levels of the biomarkers in an individual without such one or more diseases.

In some embodiments the invention provides a method of preventing, treating, and/or alleviating symptoms associated with one or more diseases by administering to a subject in need thereof one or more polypeptides or the compositions of the above aspects of the invention. In some embodiments, one or more diseases can be Parkinson's disease, Alzheimer's disease, AIDS, Lyme disease, malaria, SARS, Down syndrome, anthrax, bacterial botulism. In some embodiments, one or more polypeptides or the compositions of the above aspects may further include one or more entities selected from the group consisting of therapeutic agent, toxin, and radionucleotide.

In some embodiments, the invention provides a method of simultaneously diagnosing, preventing, treating, and/or alleviating symptoms associated with an individual. The method includes a) administering to the individual in need thereof the polypeptide or the composition of the above aspects of the invention, b) detecting the presence or absence of a biomarker for the disease in which the detection includes utilizing the polypeptide or the composition of the above aspects of the invention in which the polypeptide or composition binds specifically to a biomarker associated with the disease, c) determining the level of the biomarker if present in the individual's sample, d) comparing the level to a reference value, e) identifying the individual as having the disease when the level of the biomarker in the individual's sample is higher than the reference value, and f) preventing, treating, and/or alleviating symptoms associated with the disease in the individual when the polypeptide or the composition of the above aspects of the invention specifically binds to the biomarker. In one embodiment, the polypeptide or the composition may further include an entity selected from the group consisting of therapeutic agent, toxin, and radionucleotide. In some embodiments, the reference value is the level of the biomarker in an individual without such disease. In some embodiments, the reference value is the level of the biomarker in the same individual measured at a different time. In some embodiments, the reference value is the level of the biomarker from a collected pool of samples from different individuals.

In some embodiments, the disease may be cancer, Parkinson's disease, Alzheimer's disease, AIDS, Lyme disease, malaria, SARS, Down syndrome, anthrax, salmonella or bacterial botulism, staphylococcus aureus. In some embodiments, the cancer can be lung cancer, bladder cancer, gastric cancer, ovarian cancer, brain cancer, breast cancer, prostate cancer, cervical cancer, ovarian cancer, oral cancer, colorectal cancer, leukemia, childhood neuroblastoma, or Non-Hodgkin's lymphoma.

In some embodiments, the biomarker can be AMACR, TMPRSS2-ERG, HAAH, APP, A1342, ALZAS, Tau, gamma secretase, beta secretase, PEDF, BDNF, Cystatin C, VGF nerve growth factor inducible, APO-E, GSK-3 binding protein, TEM1, PGD2, EGFR, ESR-1, HER-2/neu, P53, RAS, SMAD4, Smad7, TNF-alfa, HPV, tPA, PCA-3, Mucin, Cadherin-2, FcRn alpha chain, cytokerratin 1-20, Apo-H, Celuloplasmin, Apo AII, VGF, Vif, LEDGF/p75, TS101, gp120, CCR5, HIV protease, HIV integrase, Bacillus anthracis protein, NadD (Nicotinate Mononucletide Adenyltransferase), Plasmodium falciparum cGMP directed phosphodiestrase, chain B of Clostridium botulinum neurotroxin type E protein, Borrelia VlsE protein, ACE2 receptor, SFRS4, or SAMP.

In some embodiments, the biomarkers for Alzheimer's disease may be Amyloid-bet, ALZAS, Tau, DJ-1, Bax-1, PEDF, HPX, Cystatin-C, Beta-2-Microglobulin, BDNF, Tau-Kinase, gamma-Sercretase, beta-Secretase, Apo-E4, and VGF-Peptide.

In some embodiments, the biomarkers associated with Parkinson's Disease may be Apo-H, Cerulopasmin, Chromogranin-B, VDBP, Apo-E, Apo-AII, and alaf-Synuclein.

In some embodiments, the biomarkers for Brain Cancer may be TEM1, Plasmalemmal Vesicle (PV-1), Prostaglandin D Synthetase, and (PGD-S).

In some embodiments, the biomarkers for HIV/AIDS, wherein said biomarkers for HIV/AIDS may be gp120, Vif, LEDGF/p75, TS101, HIV-Integrase, HIV-Reverse Transcriptase, HIV-Protease, CCR5, and CXCR4.

In some embodiments, the biomarkers for Lung Cancer may be KRAS, Ki67, EGFR, KLKB1, EpCAM, CYFRA21-1, tPA, ProGRP, Neuron-specific Enolase (NSE), and hnRNP.

In some embodiments, the biomarkers for Prostate Cancer may be AMACR, PCA3, TMPRSS2-ERG, HEPSIN, B7-H3, SSeCKs, EPCA-2; PSMA, BAG-1, PSA, MUC6, hK2, PCA-1, PCNA, RKIP, and c-HGK.

In some embodiments, the biomarkers for Breast Cancer may be EGFR, EGFRT790M, HER-2, Notch-4, ALDH-1, ESR1, SBEM, HSP70, hK-10, MSA, p53, MMP-2, PTEN, Pepsinigen-C, Sigma-S, Topo-11-alfauKPA, BRCA-1, BRCA-2, SCGB2A1, and SCGB1D2.

In some embodiments, the biomarkers for Colorectal Cancer may be SMAD4, EGFR, KRAS, p53, TS, MSI-H, REGIA, EXTL3, p1K3CA, VEGF, HAAH, EpCAM, TEM8, TK1, STAT-3, SMAD-7, beta-Catenin, CK20, MMP-1, MMP-2, MMP-7, 9, 11, and VEGF-D.

In some embodiments, the biomarkers for Ovarian Cancer may be CD24, CD34, EpCAM, hK8, 10, 13, CKB, Cathesin B, M-CAM, c-ETS1, and EMMPRIN.

In some embodiments, the biomarkers for Cervical Cancer may be HPV, CD34, ERCC1, Beta-CF, Id-1, UGF, SCC, p16, p21WAF1, PP-4, and TPS.

In some embodiments, the biomarkers for Bladder Cancer may be CK18, CK20, BLCa-1, BLCA-4, CYFRA21-1, TFT, BTA, Survivin, UCA1, UPII, FAS, and DD23.

In some embodiments, the bacteria or biomarkers associated with a disease causing bacteria can be Clostridium Botulinum (Bacterial Botulism), Bacillus Anthracis (Anthrax), Salmonella Typhi (Typhoid Fever), Treponema Pallidum (Syphilis), Plasmodinum (Malaria), Chlamadyia (STDs), Borrelia B (Lyme disease), Staphyloccus Aureus, Tetanus, Meningococcal Meningitis (Bacterial Meningitis), and Mycobacterium tuberculosis (Tuberculosis, TB), and NadD (Nicotinate Mononucleotide Adenyltransferase, an enzyme involved in inducing resistance to antibiotics);

In some embodiments, disease causing virus or biomarkers associated may be Pandemic Flu Virus H1N1 strain, Influenza virus H5N1 strain, Hepatitis B virus (HBV) antigen OSt-577, HBV core antigen HBcAg (HBV), HBV antigen Wnt-1, Hepatitis C Virus (HCV) antigen Wnt-1, and HCV RNA (HCV).

In some embodiments, a nucleic acid encodes all or portion of a polypeptide having all or a portion of at least one variable (Vab) domain of camelid and or shark single domain heavy chain antibody, all or a portion of at least one constant domain 1 (CH1) of human heavy chain immunoglobulin G in which the polypeptide includes at least one binding site for an antigen. In one embodiment, the polypeptide includes at least two variable (Vab) domains of camelid and or shark single domain heavy chain antibody. In another embodiment, the polypeptide includes at least three, at least four or more variable (Vab) domains of camelid and or shark single domain heavy chain antibody.

In one embodiment, the nucleic acid is operably linked to one or more expression regulatory element which is capable of modulating the expression of the nucleic acid. Exemplary expression regulatory elements include but are not limited to a promoter, enhancer, 5′- and 3′-untranslated regions, polyadenylation signal.

In some embodiments, the invention provides a method for producing a polypeptide of the above aspects of the invention. The method includes transforming a host cell with a recombinant nucleic acid encoding the polypeptide of the above aspects of the invention, and expressing the polypeptide in the host cell. In one embodiment, the host cell is eukaryotic. In another embodiment, the host cell is prokaryotic.

In another embodiment, the invention provides a method for producing a polypeptide of the above aspects of the invention. The method includes a chemical synthesis of a polypeptide comprising one, two, or more variable antigen-binding (Vab) domains using the parent antibody produced from camelid and/or shark as a starting material for generating the polypeptide with one or more Vab domains. Still in another embodiment, the invention provides a method for generating polypeptides comprising multivalent variable antigen-binding domains improving binding affinity between antibody and its antigen, and to improve it biodistribution and retention. Biological molecules with molecular weight between 15 to 17 KDa, though can enter a cell or cross blood brain barrier (BBB), they are not retained inside the cell to be therapeutically efficacious [Nature Biotechnology, 23, 1126 (2005)]. Conversely, biologicals, such as, conventional mouse monoclonal antibodies are too big (MW ˜150 KDa) to enter a cell or cross BBB efficiently. Ideal tumor targeting reagents are intermediate-sized multivalent molecules with molecular weight of ˜55 KDa [Nature Biotechnology, 23, 1126 (2005)]. In one embodiment, the invention encompasses the synthesis of a polypeptide with two or more variable antigen-binding domains to generate the polypeptide with a MW ˜30 to 60 KDa, more preferably 40 to 60 KDa, but ideally ˜55 KDa. The polypeptide comprises camelid Vab domains and/or shark V-NAR domains, in which such constructions/preparations are performed either chemically and/or via recombinant DNA methods.

In some embodiments of the above aspects, the invention provides a method for detecting the presence or absence of an antigen associated with a disease in a sample. The method includes a) obtaining a sample suspected of having the antigen, b) detecting the level of the antigen in the sample utilizing the polypeptide or composition of the above aspects of the invention in which the polypeptide or composition binds specifically to the antigen. The level of the antigen in the sample is indicative of the presence or absence of the antigen.

In some embodiments of the above aspects, the invention provides a method for detecting the presence or absence of circulating tumor cells in a sample. The method includes a) obtaining a sample suspected of having circulating tumor cells, b) detecting the level of one or more tumor cell surface receptors utilizing the polypeptide or composition of the above aspects of the invention in which the polypeptide or composition binds specifically to the tumor cell surface receptors. The level of the tumor cell surface receptors in the sample is indicative of the presence or absence of the circulating tumor cell. Exemplary tumor cell surface receptors include but not limited to MUC-1, VCAM-1, EpCAm-1, CD44, CD133, E-Cadherin, VEGF, bFGF, sFASL, CD95, p53, Bcl-2 CyclinD1, Cyclin E, TNF-alfa, TGF-beta1, Her-2, EGFR, IGF-1 and IGF-1R, IL-2R, Ras, and cMyc.

In some embodiments of the above aspects, the invention provides a method for detecting the presence or absence of circulating fetal cells in a sample. The method includes a) obtaining a sample suspected of having circulating fetal cells, b) detecting the level of one or more fetal cell surface receptors utilizing the polypeptide or composition of the above aspects of the invention in which the polypeptide or composition binds specifically to the fetal cell surface receptors. The level of the fetal cell surface receptors in the sample is indicative of the presence or absence of the circulating fetal cell. Exemplary tumor cell surface receptors include but not limited to GPA, CD71, CD133, CD34, CD44, ITCAM, ITGB1 (Integrin beta-1), Trop-1, Trop-2, HLA-G233, and 6B5.

In some embodiments, the invention provides a method for detecting an organism or a cell. The method includes obtaining a sample, detecting the presence or absence of one or biomarkers associated with the organism or a cell utilizing the polypeptides or compositions of the above aspects of the invention. The presence of one or more biomarkers in the sample is indicative of the presence of the organism or a cell. In some embodiments, the organism is a pathogenic organism such as bacteria or virus. In some embodiments, the pathogenic organism is selected from the group consisting of Bacillus anthracis, Borrelia burgdorferi, Salmonella typhi, Plasmodium falciparum, Human immune deficiency virus (HIV), Hepatitis B virus (HBV), and severe acute respiratory syndrome virus (SARS). In some embodiments, the cell is selected from the group consisting of circulating fetal cell and circulating tumor cell.

The term “antibody” as used herein refers to immunoglobulin G (IgG) having only heavy chains without the heavy chain constant domain 1 (CH1) and also lacking the light chain such as in shark IgNAR and camelids IgG2 and IgG3. Antibody can be monoclonal or polyclonal.

The term “analog” within the scope of the term “antibody” include those produced by digestion with various proteases, those produced by chemical cleavage, chemical coupling, chemical conjugation, and those produced recombinantly, so long as the fragment remains capable of specific binding to a target molecule. Analogs within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

The terms “heavy chain only antibody” and “single domain heavy chain antibody” has been used herein interchangeably in the context of camelid and shark antibodies and refer to camelid immunoglobulin G (IgG) and shark IgNAR having only heavy chains without the heavy chain constant domain 1 (CH1) and further lacking the light chain such as camelids IgG2 and IgG3 and shark IgNAR. Heavy chain only antibody can be monoclonal or polyclonal.

The term “improved biodistribution and retention” as used herein in the context of polypeptides, antibodies and its analogs refers to polypeptides, antibodies and its analogs that can cross cell membrane and blood brain barrier (BBB) and have greater thermal and chemical stability than conventional immunoglobulin G with heavy and light chains. Typically such polypeptides, antibodies and its analogs have molecular weight between 25 to 90 KDa, preferably between 30 to 60 KDa. In some embodiments, the molecular weight is at least 25 KDa, 30 KDa, 35 KDa, 40 KDa, 45 KDa, 50 KDa, 55 KDa, 60 KDa, 65 KDa, 70 KDa, 75 KDa, 80 KDa, 85 KDa, or 90 KDa. Although larger and smaller molecular weights are possible.

The term “specifically binds to” as used herein in the context of an antibody or its analogs refers to binding of an antibody or its analogs specifically to an epitope such that the antibody or its analog can distinguish between two proteins with and without such epitope.

The terms “biomarker” and antigen is used interchangeably and refer to a molecule or group of molecules comprised of nucleic acids, carbohydrates, lipids, proteins, peptides, enzymes and antibodies which is associated with a disease, physiological condition, or an organism. An organism can be pathogenic or nonpathogenic. A biomarker may not necessarily be the reason for a disease or a physiological condition. An amount of a biomarker may be increased or decreased in disease or a physiological condition.

The term “camelid” as used herein refers to members of the biological family Camelidae in the Order: Artiodactyla, Suborder: Tylopoda. Exemplary members of this group include camels, dromedaries, llamas, alpacas, vicuñas, and guanacos.

The term “shark” as used herein refers to members that belong to the super order Selachimorpha in the subclass Elasmobranchii in the class Chondrichthyes. There are more than 400 species of sharks known. Exemplary members of the class Chondrichthyes include great white sharks, houndsharks, cat sharks, hammerhead sharks, blue, tiger, bull, grey reef, blacktip reef, Caribbean reef, blacktail reef, whitetip reef, oceanic whitetip sharks, zebra sharks, nurse sharks, wobbegongs, bramble sharks, dogfish, roughsharks, and prickly sharks.

The term “a portion of” in the context of antibodies such as camelid and shark heavy chain only antibodies and their analogs, or human antibodies means at least 2, 5, 10, 15, 20, 25, 30, 35, 40, 45, 50, 75, 100, 125, 150, 200, 250, 300, 350, 400 or more amino acids.

The term “a portion of” in the context of hinge region of camelid and shark single domain heavy chain antibodies means at least 1, 2, 5, 15, 20, 25, 30, 35, 40, 45, 50, 75, 100, 125, 150, 200, 250, 300, 350, 400 or more amino acids of the hinge region.

The terms “diagnose” or “diagnosis” as used herein refers to the act or process of identifying or determining a disease or condition in an organism or the cause of a disease or condition by the evaluation of the signs and symptoms of the disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease.

The term “reference value” as used herein means a value which can be used for comparison with a biomarker under investigation. In one case, a reference value may be the level of a biomarker under investigation from one or more individuals without any known disease. In another case, a reference value may be the level of the biomarker in an individual's sample collected at a different time.

The terms “treatment,” “treating,” or “treat” as used herein refers to care by procedures or application that are intended to relieve illness or injury. Although it is preferred that treating a condition or disease will result in an improvement of the condition, the term treating as used herein does not indicate, imply, or require that the procedures or applications are at all successful in ameliorating symptoms associated with any particular condition. Treating a patient may result in adverse side effects or even a worsening of the condition which the treatment was intended to improve.

“Sample” or “patient sample” as used herein includes biological samples such as cells, tissues, bodily fluids, and stool. “Bodily fluids” may include, but are not limited to, blood, serum, plasma, saliva, cerebral spinal fluid, pleural fluid, tears, lactal duct fluid, lymph, sputum, urine, amniotic fluid, and semen. A sample may include a bodily fluid that is “acellular”. An “acellular bodily fluid” includes less than about 1% (w/w) whole cellular material. Plasma or serum are examples of acellular bodily fluids. A sample may include a specimen of natural or synthetic origin.

The term “body fluid” or “bodily fluid” as used herein refers to any fluid from the body of an animal. Examples of body fluids include, but are not limited to, plasma, serum, blood, lymphatic fluid, cerebrospinal fluid, synovial fluid, urine, saliva, mucous, phlegm and sputum. A body fluid sample may be collected by any suitable method. The body fluid sample may be used immediately or may be stored for later use. Any suitable storage method known in the art may be used to store the body fluid sample; for example, the sample may be frozen at about −20° C. to about −70° C. Suitable body fluids are acellular fluids. “Acellular” fluids include body fluid samples in which cells are absent or are present in such low amounts that the peptidase activity level determined reflects its level in the liquid portion of the sample, rather than in the cellular portion. Typically, an acellular body fluid contains no intact cells. Examples of acellular fluids include plasma or serum, or body fluids from which cells have been removed.

The term “enzyme linked immunosorbent assay” (ELISA) as used herein refers to an antibody-based assay in which detection of the antigen of interest is accomplished via an enzymatic reaction producing a detectable signal. ELISA can be run as a competitive or non-competitive format. ELISA also includes a 2-site or “sandwich” assay in which two antibodies to the antigen are used, one antibody to capture the antigen and one labeled with an enzyme or other detectable label to detect captured antibody-antigen complex. In a typical 2-site ELISA, the antigen has at least one epitope to which unlabeled antibody and an enzyme-linked antibody can bind with high affinity. An antigen can thus be affinity captured and detected using an enzyme-linked antibody. Typical enzymes of choice include alkaline phosphatase or horseradish peroxidase, both of which generated a detectable product upon digestion of appropriate substrates.

The term “label” as used herein, refers to any physical molecule directly or indirectly associated with a specific binding agent or antigen which provides a means for detection for that antibody or antigen. A “detectable label” as used herein refers any moiety used to achieve signal to measure the amount of complex formation between a target and a binding agent. These labels are detectable by spectroscopic, photochemical, biochemical, immunochemical, electromagnetic, radiochemical, or chemical means, such as fluorescence, chemifluoresence, or chemiluminescence, electro-chemiluminescence or any other appropriate means. Suitable detectable labels include fluorescent dye molecules or fluorophores.

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

The term “detectable label” as used herein in the context of antibody or its analogs refers to a molecule or a compound or a group of molecules or a group of compounds associated with a binding agent such as an antibody or its analogs, secondary antibody and is used to identify the binding agent bound to its target such as an antigen, primary antibody. A detectable label can also be used in to detect nucleic acids. In such cases a detectable label may be incorporated into a nucleic acid during amplification reactions or a detectable label may be associated a probe to detect the nucleic acid.

“Detecting” as used herein in context of detecting a signal from a detectable label to indicate the presence of a nucleic acid of interest in the sample (or the presence or absence of a protein of interest in the sample) does not require the method to provide 100% sensitivity and/or 100% specificity. As is well known, “sensitivity” is the probability that a test is positive, given that the person has a genomic nucleic acid sequence, while “specificity” is the probability that a test is negative, given that the person does not have the genomic nucleic acid sequence. A sensitivity of at least 50% is preferred, although sensitivities of at least 60%, at least 70%, at least 80%, at least 90% and at least 99% are clearly more preferred. A specificity of at least 50% is preferred, although specificity of at least 60%, at least 70%, at least 80%, at least 90% and at least 99% are clearly more preferred. Detecting also encompasses assays with false positives and false negatives. False negative rates may be 1%, 5%, 10%, 15%, 20% or even higher. False positive rates may be 1%, 5%, 10%, 15%, 20% or even higher.

The term “about” as used herein in reference to quantitative measurements or values, refers to the indicated value plus or minus 10%.

“Nucleic acid” as used herein refers to an oligonucleotide, nucleotide or polynucleotide, and fragments or portions thereof, which may be single or double stranded, and represent the sense or antisense strand. A nucleic acid may include DNA or RNA, and may be of natural or synthetic origin and may contain deoxyribonucleotides, ribonucleotides, or nucleotide analogs in any combination.

Non-limiting examples of polynucleotides include a gene or gene fragment, genomic DNA, exons, introns, mRNA, tRNA, rRNA, ribozymes, cDNA, recombinant polynucleotides, branched polynucleotides, plasmids, vectors, isolated DNA of any sequence, isolated RNA of any sequence, synthetic nucleic acid, nucleic acid probes and primers. Polynucleotides may be natural or synthetic. Polynucleotide may comprise modified nucleotides, such as methylated nucleotides and nucleotide analogs, uracyl, other sugars and linking groups such as fluororibose and thiolate, and nucleotide branches. A nucleic acid may be modified such as by conjugation, with a labeling component. Other types of modifications included in this definition are caps, substitution of one or more of the naturally occurring nucleotides with an analog, and introduction of chemical entities for attaching the polynucleotide to other molecules such as proteins, metal ions, labeling components, other polynucleotides or a solid support. Nucleic acid may include nucleic acid that has been amplified (e.g., using polymerase chain reaction).

A fragment of a nucleic acid generally contains at least about 15, 20, 25, 30, 35, 40, 45, 50, 75, 100, 200, 300, 400, 500, 1000 nucleotides or more. Larger fragments are possible and may include about 2,000, 2,500, 3,000, 3,500, 4,000, 5,000 7,500, or 10,000 bases.

“Gene” as used herein refers to a DNA sequence that comprises control and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained.

“cDNA” as used herein refers to complementary or copy polynucleotide produced from an RNA template by the action of RNA-dependent DNA polymerase activity (e.g., reverse transcriptase). cDNA can be single stranded, double stranded or partially double stranded. cDNA may contain unnatural nucleotides. cDNA can be modified after being synthesized. cDNA may comprise a detectable label.

As used herein, “subject” or “individual” is meant a human or any other animal that has cells. A subject can be a patient, which refers to a human presenting to a medical provider for diagnosis or treatment of a disease. A human includes pre and post natal forms.

The term “patient” as used herein, refers to one who receives medical care, attention or treatment. As used herein, the term is meant to encompass a person diagnosed with a disease as well as a person who may be symptomatic for a disease but who has not yet been diagnosed.

The term “vector” as used herein refers to a recombinant DNA or RNA plasmid or virus that comprises a heterologous polynucleotide capable of being delivered to a target cell, either in vitro, in vivo or ex-vivo. The heterologous polynucleotide can comprise a sequence of interest and can be operably linked to another nucleic acid sequence such as promoter or enhancer and may control the transcription of the nucleic acid sequence of interest. As used herein, a vector need not be capable of replication in the ultimate target cell or subject. The term vector may include expression vector and cloning vector.

Suitable expression vectors are well-known in the art, and include vectors capable of expressing a polynucleotide operatively linked to a regulatory sequence, such as a promoter region that is capable of regulating expression of such DNA. Thus, an expression vector refers to a recombinant DNA or RNA construct, such as a plasmid, a phage, recombinant virus or other vector that, upon introduction into an appropriate host cell, results in expression of the inserted DNA. Appropriate expression vectors include those that are replicable in eukaryotic cells and/or prokaryotic cells and those that remain episomal or those which integrate into the host cell genome.

The term “promoter” as used herein refers to a segment of DNA that controls transcription of polynucleotide to which it is operatively linked. Promoters, depending upon the nature of the regulation, may be constitutive or regulated. Exemplary eukaryotic promoters contemplated for use in the practice of the present invention include the SV40 early promoter, the cytomegalovirus (CMV) promoter, the mouse mammary tumor virus (MMTV) steroid-inducible promoter, Moloney murine leukemia virus (MMLV) promoter. Exemplary promoters suitable for use with prokaryotic hosts include T7 promoter, beta-lactamase promoter, lactose promoter systems, alkaline phosphatase promoter, a tryptophan (trp) promoter system, and hybrid promoters such as the lac promoter.

### BRIEF DESCRIPTION OF THE FIGURES

FIG. 1 shows structural differences between camel, shark, and mouse immunoglobulins (IgGs). The notations CH2, CH3, CH4, CH5 represent constant domain 2, 3, 4 of single domain heavy chain antibody of the respective species. The notations Vab and VNAR represent variable domain of camelid and shark single domain heavy chain antibodies respectively.

FIG. 2 shows an exemplary nucleic acid sequence of variable antigen-binding region (Vab) of camel heavy chain only antibody without the light chains (SEQ ID NO: 3).

FIG. 3 shows exemplary chemical and/or protease digests of camelid and shark heavy chain only antibodies. The notation “Rn” represents all or portion of the hinge region of camelid or shark single domain antibodies.

FIGS. 4 A and B shows the structure of exemplary analogs of camelid heavy chain only antibodies: Micro-, Sub-nano-, Nano-antibodies 3-6, and Multimeric Constructs: Bivalent, 7, Trivalent, 8, Multivalent 8, Rabbit Ear-Like Bivalent Construct 9 and Bivalent Construct 10 with and without Val37, Gly44, Leu45, and Trp47 of Vab changed to polar hydrophilic amino acids, namely, Ser, Gln, Tyr., His, Asn, Thr, Asp, Cys, Glu, Lys, and Arg. The notation “Rn” represents all or portion of the hinge region of camelid or shark single domain antibodies.

FIG. 5 shows an exemplary scheme of cloning and expression of camelid heavy chain only antibodies and their analogs.

FIG. 6 shows an exemplary nucleic acid sequence of constant domain 1 (CH1) of human IgG (SEQ ID NO: 7).

FIG. 7 shows an exemplary scheme of making recombinant bivalent analog of camelid heavy chain only antibodies.

FIG. 8 shows an exemplary scheme of chemical synthesis of bivalent analog of camelid and shark heavy chain only antibodies. The notation “Rn” represents all or portion of the hinge region of camelid or shark single domain antibodies.

FIG. 9 shows an alternative exemplary scheme of chemical synthesis of bivalent analog of camelid heavy chain only antibodies. The notation “Rn” represents all or portion of the hinge region of camelid or shark single domain antibodies.

FIG. 10 shows an exemplary scheme of chemical synthesis of multivalent analog of camelid and shark heavy chain only antibodies. The notation “Rn” represents all or portion of the hinge region of camelid or shark single domain antibodies.

FIG. 11 shows an exemplary scheme of chemical synthesis of trivalent analog of camelid and shark heavy chain only antibodies. The notation “Rn” represents all or portion of the hinge region of camelid or shark single domain antibodies.

FIGS. 12 A and B shows exemplary camelid heavy-chain only antibodies and its analogs conjugated to various entities for diagnostic and therapeutic applications. Man stands for maleic anhydride. The notation “Rn” represents all or portion of the hinge region of camelid or shark single domain antibodies.

FIG. 13 shows the exemplary pegylation scheme of camelid heavy-chain only antibodies and its analogs to yield the corresponding pegylated products. The notation “Rn” represents all or portion of the hinge region of camelid or shark single domain antibodies.

FIG. 14 shows an exemplary conjugation scheme of nucleic acid to camelid and shark heavy chain only antibodies and their analogs. The notation “Rn” represents all or portion of the hinge region of camelid or shark single domain antibodies.

FIG. 15 outlines the steps involved in immobilization of camelid and shark heavy chain only antibodies and their analogs to solid surfaces.

FIG. 16 shows the comparison of amino acid sequences of VH domains of conventional monoclonal antibody (mAbVH) with Vab (cVab) region of camel IgG3, Vab region of camel IgG2 having shorter hinge region (1Vab) and shark V-NAR.

FIG. 17 shows shark heavy chain only antibody (Structure 2), exemplary analogs (structures 52, 53, 54, 55, 56) and exemplary means of making such analogs. The notations “Rn” represents all or portion of the hinge region of camelid or shark single domain antibodies, “D” represents at least two amino acids comprising at least one charged amino acid.

FIG. 18 shows schematics of chemically making exemplary shark heavy chain only antibody analogs (structures 52-56, 67-73) and exemplary means of making conjugates with various entities. The notations “Rn” represents all or portion of the hinge region of camelid or shark single domain antibodies, “D” represents at least two amino acids comprising at least one charged amino acid.

FIG. 19 shows schematics of cloning strategy of shark heavy chain only antibody and its analogs.

FIG. 20 shows an exemplary amino acid sequence of shark IgNAR (SEQ ID NO: 16). Amino acid sequence of the various regions of the protein are underlined and indicated.

FIG. 21 shows an exemplary nucleic acid sequence of shark IgNAR (SEQ ID NO: 17).

FIG. 22 shows schematics of chemically linking exemplary hydrophilic linker to shark heavy chain only antibody analog with structure 74. The notation “Rn” represents all or portion of the hinge region of camelid or shark single domain antibodies.

FIG. 23 shows an exemplary method of making bi-valent analogs of shark heavy chain only antibody. The notation “Rn” represents all or portion of the hinge region of camelid or shark single domain antibodies.

FIG. 24 shows an exemplary method of making tetra-valent analogs of shark heavy chain only antibody. The notation “Rn” represents all or portion of the hinge region of camelid or shark single domain antibodies.

FIG. 25 shows an exemplary scheme of capturing and detecting antigens/biomarkers associated with a disease using camelid and shark heavy chain only antibodies and their analogs.

FIG. 26 shows an exemplary scheme of capturing and detecting antigens/biomarkers associated with a disease using camelid and shark heavy chain only antibodies and their analogs using immuno-PCR.

FIG. 27 shows an exemplary scheme of capturing and detecting rare cells associated with a disease using camelid and shark heavy chain only antibodies and their analogs.

FIG. 28 shows an exemplary scheme of detecting chromosomal translocation using captured circulating tumor cells using camelid and shark heavy chain only antibodies and their analogs.

FIG. 29 shows an exemplary scheme of detecting prenatal genetic disorder using captured circulating fetal cells using camelid and shark heavy chain only antibodies and their analogs.

FIG. 30 shows exemplary amino acid sequences of different biomarkers associated with a disease or pathogen (SEQ ID NOs: 48-97).

### DETAILED DESCRIPTION OF THE INVENTION

Unless otherwise specified, the terms “a” or “an” mean “one or more” throughout this application. The present invention teaches composition of camelid and/or shark single-domain heavy-chain only antibodies and their analogs for efficient cell and blood brain barrier (BBB) permeability for optimal biodistribution and retention for diagnosing and/or treating human diseases, methods for the development of nano-biomedical technology platforms utilizing camelid and/or shark heavy-chain only antibodies and their analogs for in-vitro and in-vivo diagnosis and treatment of human and animal diseases with such antibodies.

### Camelid and Shark Antibodies

The hetero-tetrameric structure exists in humans and most animals but the heavy-chain dimer structure is considered characteristic of camelids and sharks [Holliger P, Hudson P J, Nature Biotechnology, 23, 1126 (2005)]. These antibodies are relatively simple molecules but with unique characteristics. Their size is about ⅔^{rd} the size of traditional antibodies, hence a lower molecular weight (About 90 K Da), with similar antigen binding affinity, but with water solubility 100 to 1000 folds higher than the conventional antibodies. Because of the lower molecular weight, the authors of this application call these antibodies as “Heavy-Chain Mini-Antibodies” (mnHCAbs) or simply “Mini-Antibodies” (mnAbs).

Another characteristic of heavy-chain antibodies derived from sharks and camelids, for example, is that they have very high thermal stability compared to the conventional mAbs. For example, camel antibodies can maintain their antigen binding ability even at 90° C. [Biochim. Biophys. Acta., 141, 7 (1999)]. Furthermore, complementary determining region 3 (CDR3) of camel Vab region is longer, comprising of 16-21 amino acids, than the CDR3 of mouse VH region comprising of 9 amino acids [Protein Engineering, 7, 1129 (1994)]. The larger length of CDR3 of camel.Vab region is responsible for higher diversity of antibody repertoire of camel antibodies.

In addition to being devoid of light chains, the camel heavy-chain antibodies also lack the first domain of the constant region called CH1, though the shark antibodies do have CH1 domain and two additional constant domains CH4 and CH5 [Nature Biotech. 23, 1126 (2005)]. Furthermore, the hinge regions of camel and shark antibodies have an amino acid sequence different from that of normal heterotetrameric conventional antibodies [(S. Muyldermans, Reviews in Mol. Biotech., 74, 277 (2001)]. Without the light chain, these heavy-chain antibodies bind to their antigens by one single domain, the variable antigen-binding domain of the heavy-chain immunoglobulin, is referred to as Vab by the authors of this application (VHH in the literature), to distinguish it from the variable domain VH of the conventional antibodies. The single domain Vab is amazingly stable by itself without having to be attached to the heavy-chain. This smallest intact and independently functional antigen-binding fragment Vab, with a molecular weight of ˜12-15 K. Da, derived from a functional heavy-chain full length mini-immunoglobulin, is referred to as nano-antibody by the authors of this application. In the literature, it is known as nanobody [(S. Muyldermans, Reviews in Mol. Biotech., 74, 277 (2001)].

The genes encoding these full length single-domain heavy-chain antibodies and antibody-antigen binding fragment Vab (camel and shark) can be cloned in phage display vectors, and selection of antigen binders by panning and expression of selected VHH in bacteria offer a very good alternative procedure to produce these antibodies on a large scale. Also, only one domain has to be cloned and expressed to produce in vivo an intact, matured antigen-binding fragment.

There are structural differences between the variable regions of single domain antibodies and conventional antibodies. Conventional antibodies have three constant domains while camel has two and shark has five constant domains. The largest structural difference is, however, found between a VH (conventional antibodies) and Vab (heavy-chain only antibodies of camel and shark) (see below) at the hypervariable regions. Camelid Vab and shark V-NAR domains each display surface loops which are larger than for conventional murine and human IgGs, and are able to penetrate cavities in target antigens, such as enzyme active sites and canyons in viral and infectious disease biomarkers [PNAS USA., 101, 12444 (2004); Proteins, 55, 187 (2005)]. In human and mouse the VH loops are folded in a limited number of canonical structures. In contrast, the antigen binding loop of Vab possess many deviations of these canonical structures that specifically bind into such active sites, therefore, represent powerful tool to modulate biological activities [(K. Decanniere et al., Structure, 7, 361 (2000)]. The high incidence of amino acid insertions or deletions, in or adjacent to first and second antigen-binding loops of Vab will undoubtedly diversify, even further, the possible antigen-binding loop conformations.

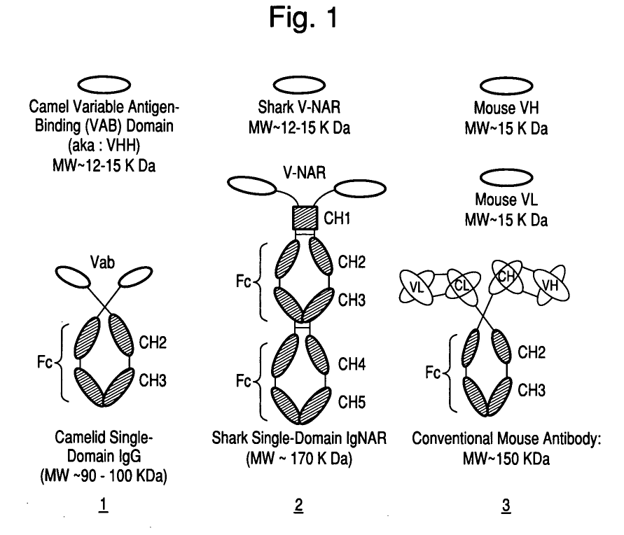

Though there are structural differences between camel and shark parent heavy-chain antibodies (FIG. 1), the antigen-antibody binding domains, Vab and V-NAR, respectively, are similar. The chemical and/or protease digestion of camel and shark antibodies results in Vab and V-NAR domains, with similar binding affinities to the target antigens [Nature Biotechnology, 23, 1126 (2005)].

Other structural differences are due to the hydrophilic amino acid residues which are scattered throughout the primary structure of Vab domain. These amino acid substitutions are, for example, Leu 45 to R (arginine) or Leu45 to C (cysteine); Val37 to Y (Tyr); G44 to E (Glu), and W47(Trp) to G (Gly). Therefore, the solubility of Vab is much higher than the Fab fragment of conventional mouse and human antibodies.

Another characteristic feature of the structure of camelid Vab and shark V-NAR is that it often contains a cysteine residue in the CDR3 in addition to cysteines that normally exist at positions 22 and 92 of the variable region. The cysteine residues in CDR3 form S—S bonds with other cysteines in the vicinity of CDR1 or CDR2 [Protein Engineering, 7, 1129 (1994)]. CDR1 and CDR2 are determined by the germline V gene. They play important roles together with CDR3 in antigenic binding [Nature Structural Biol., 9, 803 (1996); J. Mol. Biol., 311, 123 (2001)]. Like camel CDR3, shark also has elongated CDR3 regions comprising of 16-27 amino acids residues [Eur. J. Immunol., 35, 936 (2005)].

The germlines of dromedaries and llamas are classified according to the length of CDR2 and cysteine positions in the V region [Nguyen et al., EMBO J., 19, 921 (2000); Harmsen et al., Mol. Immun., 37, 579 (2000)].

Immunization of camels with enzymes generates heavy-chain antibodies (HCAb) significant proportions of which are known to act as competitive enzyme inhibitors that interact with the cavity of the active site [(M. Lauwereys et al., EMBO, J. 17, 3512 (1998)]. In contrast, the conventional antibodies that are competitive enzyme inhibitors cannot bind into large cavities on the antigen surface. Camel antibodies, therefore, recognize unique epitopes that are out of reach for conventional antibodies.

Production of inhibitory recombinant Vab that bind specifically into cavities on the surface of variety of enzymes, namely, lysozyme, carbonic anhydrase, alfa-amylase, and beta-lactamase has been achieved [M. Lauwereys, et al., EMBO, J. 17, 3512 (1998)]. Hepatitis C protease inhibitor from the camelised human VH has been isolated against an 11 amino acid sequence of the viral protease [F. Martin et al., Prot. Eng., 10, 607 (1997)].

Comparison of biological characteristics of camel antibodies, Camel VAB/Shark V-NAR, Bivalent Nano-antibodies and Mouse IgG are shown in Table 1:

TABLE 1
Comparison of Biological Characteristics of Camel, Shark V-NAR, Bivalent Nano-
Antibodies and Mouse IgG

| Biological Attribute | 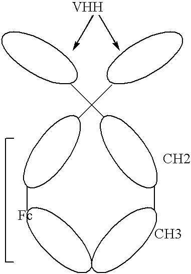 Camel Mini-antibody | 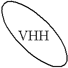 Camel VHH OR Shark V-NAR | 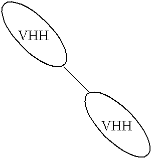 Camel Bivalent VHH | 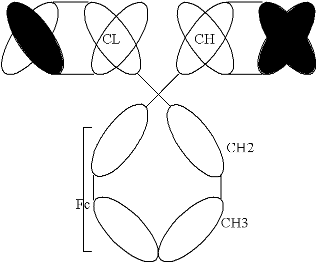 Conventional Antibody |
|---|---|---|---|---|
| MW | −90 K Da | 12-15 K Da | 24-30 K Da | 150 K Da |
| Presence of Light- | None | None | None | Two |
| Chains | | | | |
| Constant Domains | Two | Two | Two | Three |
| Binding Affinity | 10¹¹/M | 10¹¹/M | 5 × 10¹³/M | 10³/M |
| Cell Permeability | Poor | Yes | Yes | No |
| BBB Permeability | Poor | Yes | Yes | No |
| Immunogenicity | Very little to None | VLTN | VLTN | Yes |
| | (VLTN) | | | |
| Specificity | Highly Specific (HS) | HS | HS | Not very specific |
| pH Stability/Oral | 2-11/Not Known | 2-11/Yes | 2-11/Yes | 6-9/No |
| Administrability | | | | |
| Thermal Stablifty | Up to 90° C. | Up to 90° C. | Up to 90° C. | Up to 37-60° C. |
| Length of antigen- | 16 Amino- | 16 Amino- | 16 Amino- | 9 Amino- |
| binding domain | acids | acids | acids | acids |

### Camelid Heavy-Chain Antibodies, and Analogs:

Camelid heavy chain only antibodies comprise a variable antigen-binding (Vab) region, hinge region (HR), and two constant regions CH2 and CH3 as shown in FIG. 1. Exemplary amino acid sequence of camel Vab is disclosed in GenBank accession number ACF49483. The sequence is incorporated herein by reference. Exemplary camel Vab region is listed as SEQ ID NO: 1 and shown below:

(SEQ ID NO: 1)

DVQLQESGGGSVQAGGSLKLSCAISGYDNDNYCMGWFRQTPGKEREKVAA
LNIGGGSPVYADFVRGRFTISLDSSKDTLYLLMNAVTPEDTAMYYCAAIR
KPQFYTCRMWKPRADFDIWGQGTQVTVSS

Amino acid sequence of camel hinge region is disclosed in Nature 1993; 363: 446-8. Exemplary amino acid sequence of camel hinge region is listed as SEQ ID NO: 2 and shown below:

(SEQ ID NO: 2)

EPKIPQPQPKPQPQPQPQPKPQPKPEPECTCPKCP

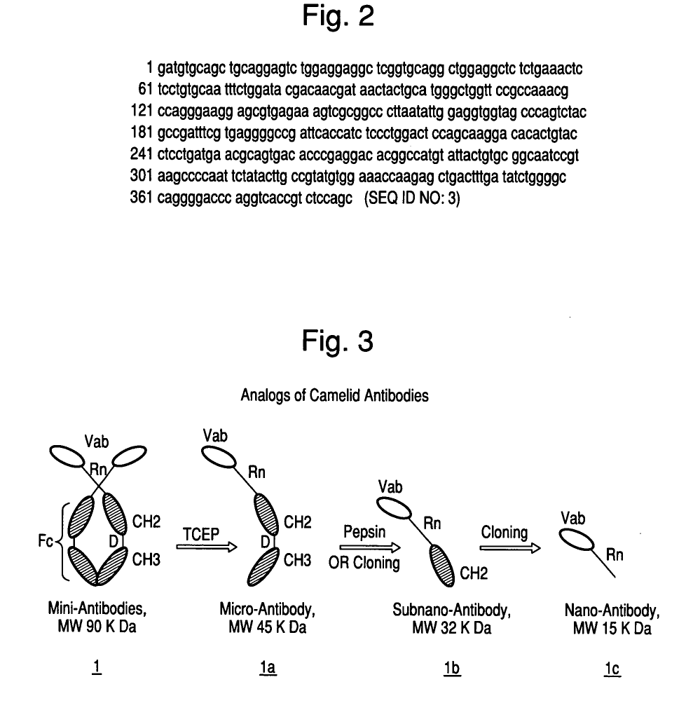

Exemplary nucleic acid sequence of camel Vab region is disclosed in GenBank accession number EU861212. Sequence of which is incorporated herein by reference. Exemplary sequence of camel Vab region is listed as SEQ ID NO: 3 and shown in FIG. 2.

Exemplary nucleic acid sequence of the hinge region of heavy chain only antibodies deduced from amino acid sequence is listed as SEQ ID NO: 4 and shown below:

(SEQ ID NO: 4)

ggacagaagacaccgcaccaacggccaagaccccacccccaacagcgacc
gcagccgagacagcggcagagacacgaaccggagtgcacgtgtcccagat
gtcc

### Production of Heavy-Chain Mini-Antibodies 1 (HCmnAbs):

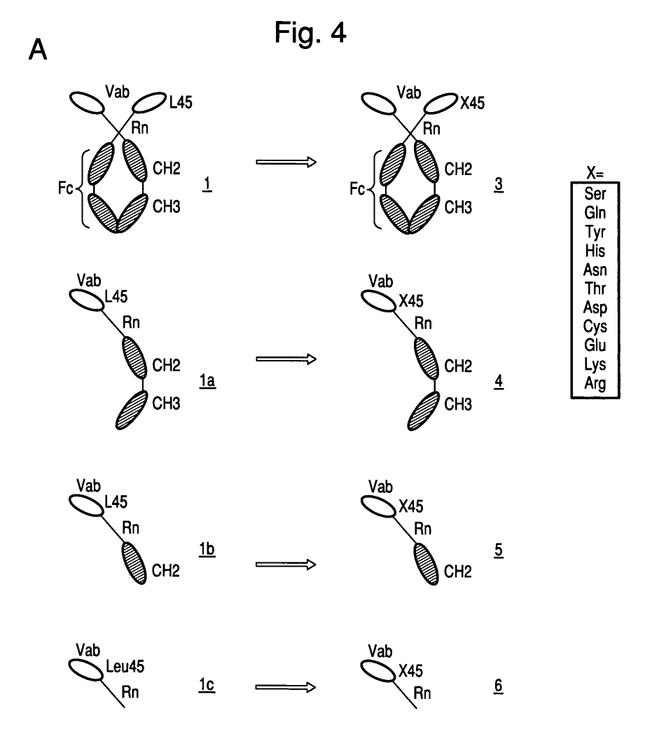

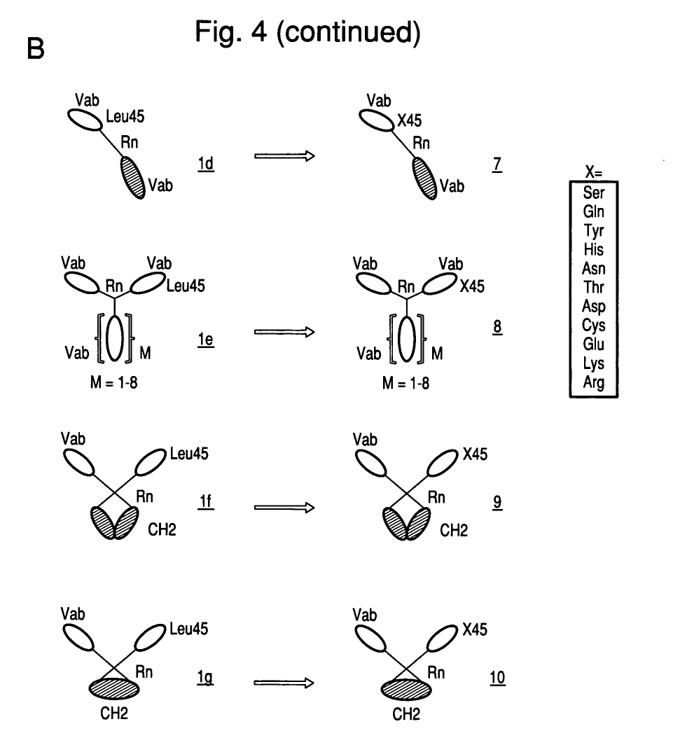

Camelids such as camels, alpacas, llamas will be immunized with one or more antigens using the biomarkers associated with different diseases and/or organisms to produce the parent antibody (HCmnAbs, Structure 1) and the mRNA, from which the variants and analogs will be derived, either chemically or through recombinant means. Exemplary analogs of camelid heavy chain only antibodies include 1a-1g, 3-14, 20-21, 23-45 and shown in FIGS. 1, 3-4, and 8-14.

### Analogs of Camelid Heavy-Chain Only Antibodies

Several analogs of camelid and shark heavy-chain only antibodies (Structure 1) are proposed in this application, particularly, to improve biodistribution and retention for optimal diagnostic and therapeutic applications. Monovalent Vab or V-NAR is of very little diagnostic and therapeutic use because it will rapidly cross cell membrane and BBB and it will also rapidly exit the tissues to be of any medicinal value. Divalent, trivalent, tetravalent and pentavalent Vab and/or V-NAR domains are the preferred analogs of this invention due to their potentially higher cellular and BBB intake and retention. The preferred analogs of the invention comprise a polypeptide with two or more variable antigen-binding domains and have a molecular weight in the range of 30-60 KDa. Exemplary analogs include but not limited to structures 1a-1g, 3-14, 20-21, 23-45 shown in FIGS. 1, 3-4, and 8-14. The analogs may be univalent or multivalent such as, divalent, trivalent, tetravalent, pentavalent etc. The analogs may be made by recombinant technology or by chemical means.

### Recombinant Production of Micro, Sub-Nano- and Nano-Antibodies 1a, 1b, and 1c

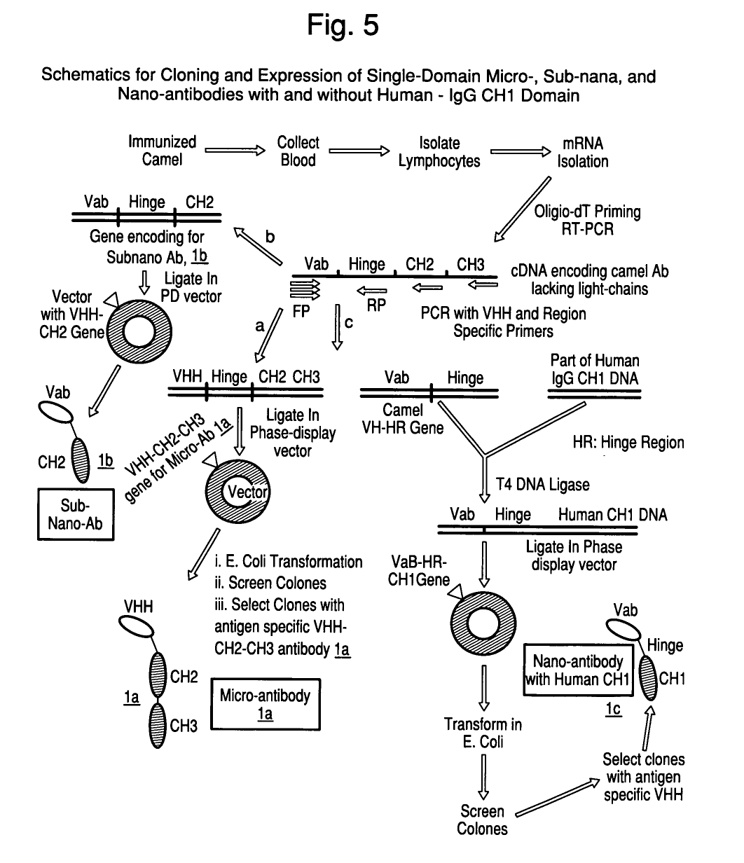

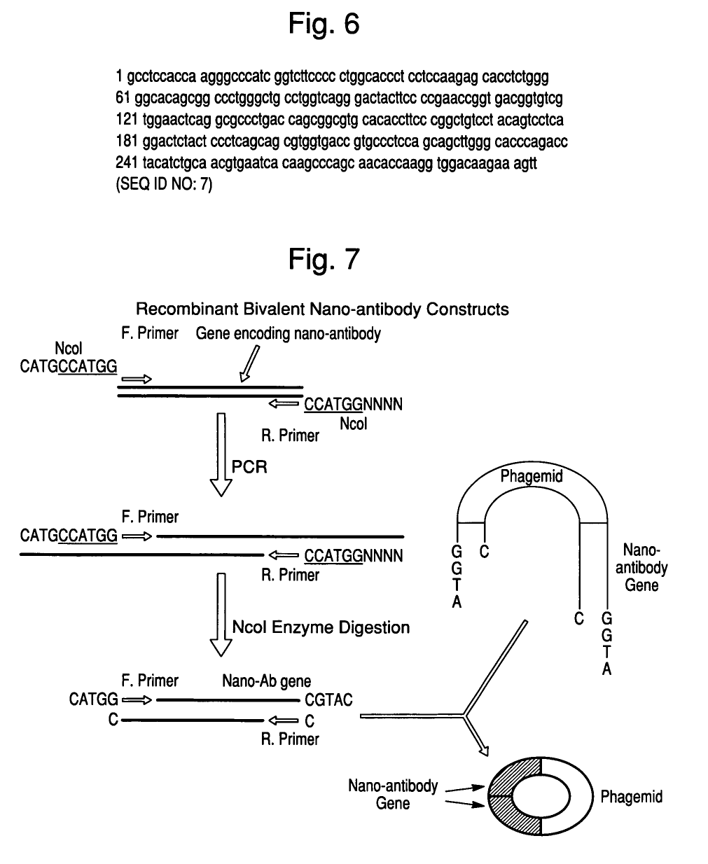

The steps involved in the production of various analogs of camelid single-domain antibodies 1a, 1b, and 1c with and without the constant domain 1 (CH1) of human IgG are outlined in FIG. 5. Exemplary nucleic acid sequence of CH1 domain of human IgG is disclosed in GenBank accession number E01508. Sequences of which are incorporated herein by reference. Exemplary nucleic acid sequence of CH1 domain of human IgG is listed as SEQ ID NO: 7 and shown in FIG. 6.

Briefly, mRNA from camelid species will be isolated using commercially available kits for example, RNeasy Protect Mini kit, RNeasy Protect Cell Mini kit, QIAamp RNA Blood Mini kit, RNeasy Protect Saliva Mini kit, Paxgene Blood RNA kit from Qiagen; MELT™, RNaqueous®, ToTALLY RNA™, RiboPure™-Blood, Poly(A)Purist™ from Applied Biosystems; TRIZOL® reagent, Dynabeads® mRNA direct kit from Invitrogen.

Nucleic acid extracted can be amplified using nucleic acid amplification techniques well know in the art. Nucleic acid amplification can be linear or exponential. By way of example, but not by way of limitation these techniques can include the polymerase chain reaction (PCR) and reverse transcriptase polymerase chain reaction (RT-PCR).

Oligonucleotide primers for use in these methods can be designed according to general guidance well known in the art as described herein, as well as with specific requirements as described herein for each step of the particular methods described.

In some embodiments, oligonucleotide primers for cDNA synthesis and PCR are 10 to 100 nucleotides in length, preferably between about 15 and about 60 nucleotides in length, more preferably 25 and about 50 nucleotides in length, and most preferably between about 25 and about 40 nucleotides in length. There is no standard length for optimal hybridization or polymerase chain reaction amplification.

Methods of designing primers have been described in U.S. patent application Ser. No. 10/921,482. Primers useful in the methods described herein are also designed to have a particular melting temperature (Tₘ) by the method of melting temperature estimation. Commercial programs, including Oligo™, Primer Design and programs available on the interne, including Primer3 and Oligo Calculator can be used to calculate a Tₘ of a polynucleotide sequence useful according to the invention.

Tₘ of a polynucleotide affects its hybridization to another polynucleotide (e.g., the annealing of an oligonucleotide primer to a template polynucleotide). In the subject methods, it is preferred that the oligonucleotide primer selectively hybridizes to a target template or polynucleotides derived from the target template (i.e., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In preferred embodiments, 100% complementarity is preferred.

Portions of CH1 domain of human IgG can be synthesized. Exemplary sequences include SEQ ID NO: 8-15. Restriction enzyme sites (such as Xho 1) can be designed at the 3′-end of the sequence. Sequences of SEQ ID NO: 8-15 are shown below. The Xho restriction site is underlined.

(SEQ ID NO: 8)

5′-ctc gag gcc tcc acc aag ggc ctc gag-3′

(SEQ ID NO: 9)

5′-gcctccaccaagggcccatc ggtcttcccc-3′

(SEQ ID NO: 10)

5′-gcctccaccaagggcccatcggtcttccccctggcaccctcc-3′

(SEQ ID NO: 11)

5′-gcctccaccaagggcccatcggtcttccccctggcaccctcctccaa
gag cacctctggg-3′

(SEQ ID NO: 12)

5′-ggcacagcgg ccctgggctg cctggtcagg gactacttcc
ccgaaccggt gacggtgtcg-3′

(SEQ ID NO: 13)

5′-tggaactcag gcgccctgac cagcggcgtg cacaccttcc
cggctgtcct acagtcctca-3′

(SEQ ID NO: 14)

5′-ggactctact ccctcagcag cgtggtgacc gtgccctcca
gcagcttggg cacccagacc-3′

(SEQ ID NO: 15)

5′-tacatctgca acgtgaatca caagcccagc aacaccaagg
tggacaagaa agtt-3′

Portions of CH1 domain of human IgG can be blunt end ligated to the 3′-end of camelid Vab domain sequence using methods well known in the art. See, Sambrook et al., Molecular Cloning: A Laboratory Manual (2nd Ed.) (1989), Cold Spring Harbor Press, N.Y. The ligated product will be analyzed and purified by agarose gel. The ligated product may be inserted into phage display vectors using standard methods. See, Sambrook et al., Molecular Cloning: A Laboratory Manual (2nd Ed.) (1989), Cold Spring Harbor Press, N.Y.

Plasmid library will be constructed using the PCR amplicons comprising Vab domains and all possible permutation and combination of all or portions of Hinge region (HR), constant domain 1 of heavy chain of human IgG (CH1), constant domain 2 of camelid or the constant domain 1 (CH1) shark heavy chain only antibody, or constant domain 2 CH2, constant domain 3 of camelid or shark heavy chain only antibody (CH3). In some embodiments, the amplicons may include domains from at least two different species such camelid and shark, or two different camelid species such as llama, camel, alpaca. Exemplary amplicons include but not limited to Vab-HR-CH2-CH3, Vab-HR, Vab-HR-CH1, Vab-CH2-CH3+AA 45 (amino acid 45 is hydrophilic amino acids such as, Lys, His, Ser, Asn, Gln, Arg, Gln, Glu, Cys, Asp or Thr), Vab-HR-CH1+AA 45, Vab-HR+AA 45, Vab-HR-Vab, Vab-HR-CH1-Vab, Vab-HR-CH2-Vab, Vab-HR-Vab-HR-Vab, Vab-HR-Vab-HR-Vab-HR-Vab.

### Shark Antibodies and Analogs:

Sharks produce multiple IgG classes including heavy-chain only antibodies missing light-chains [Comp. Biochem. Physiol. B., 15, 225 (1973)]. Shark heavy-chain only antibodies are also known as immunoglobulins new antigen receptors (IgNAR), their variable domain is designated as V-NAR. The CDR3 region of V-NAR domain is also significantly longer than that of conventional VH domains [Med. Microbiol. Immunol., 198, 157 (2009)].

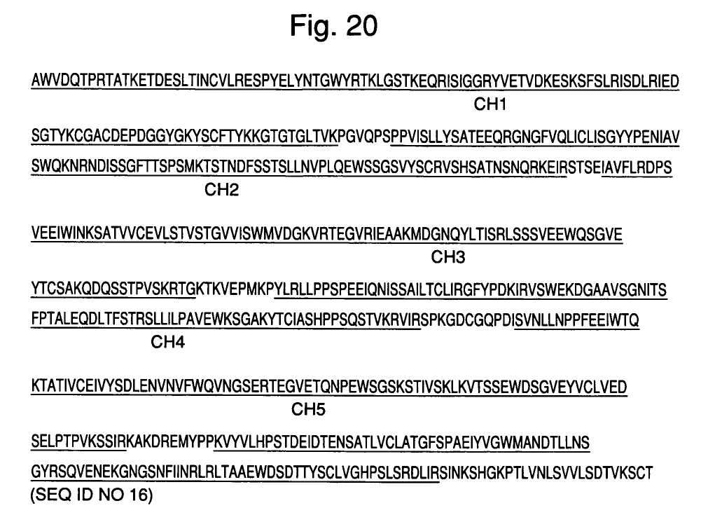

The Ig-NAR protein has been found to be a dimer with each chain composed of one variable (V) and five constant (C) domains and shown as structure 2 in FIG. 1. The V regions of NAR proteins conforms to the model of prototypic Ig superfamily domains with the predicted canonical disulfide bond connecting two beta sheets and several other invariants or conserved residues involved in the structural packing. The V-NAR region has been found to be unique in that it has an exceptionally small CDR2 and poor conservation of those amino acid residues responsible for VH/VL domain in typical IgGs and T-cell receptors. Exemplary amino acid sequence of shark Ig-NAR is disclosed in GenBank accession number ABB83616. Sequence of which is incorporated herein by reference. Exemplary amino acid sequence of shark Ig-NAR is listed as SEQ ID NO: 16 and shown in FIG. 20.

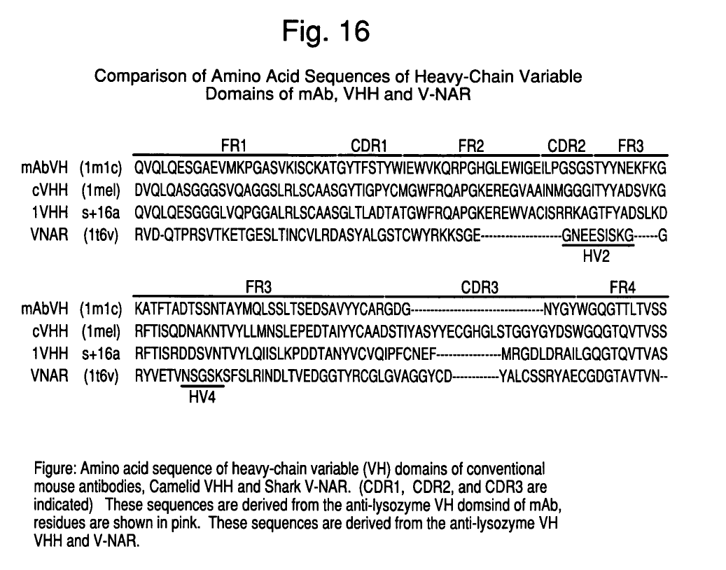

The comparison of amino acid sequences of VH domains of conventional monoclonal antibody (mAb) with camel Vab and shark V-NAR is shown in the FIG. 16.

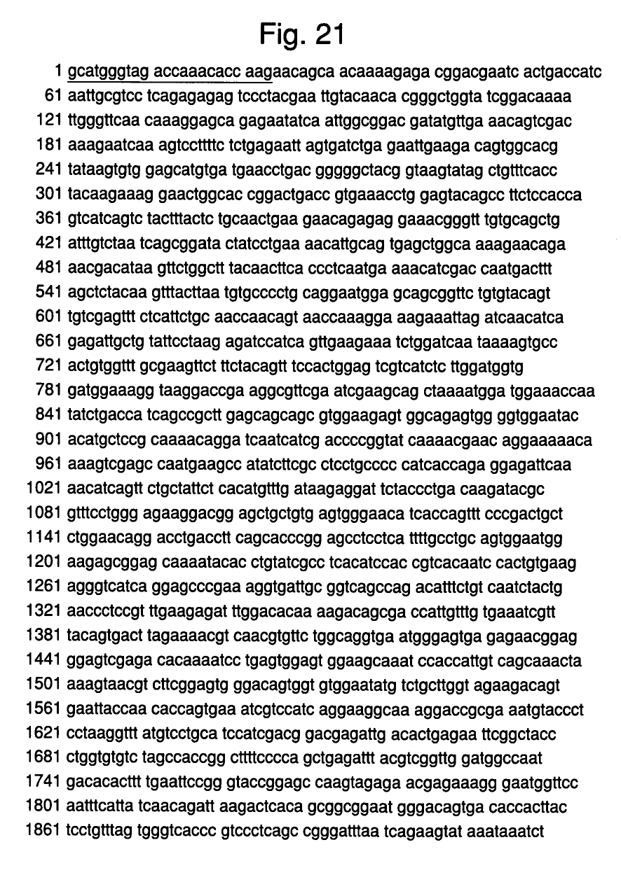

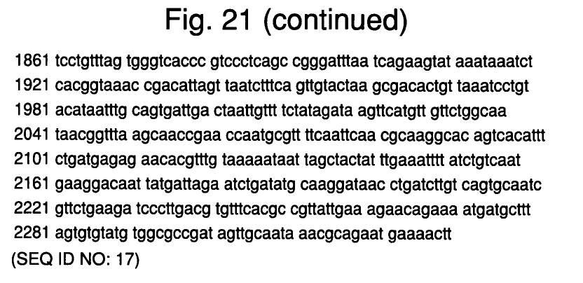

Exemplary nucleic acid sequence of shark IgNAR is disclosed in GenBank accession number DQ268538. Sequence of which is incorporated herein by reference. Exemplary sequence of shark IgNAR is listed as SEQ ID NO: 17 and shown in FIG. 21.

There are striking similarities in the structures of camelid and shark antibodies, in particular the variable regions (see structures 1 and 2) and shown in FIG. 16. Just like Vab of camel antibodies, V-NAR of shark antibodies is also independently stable and functionally active without being attached to the parent antibody. It is advantageous to develop and evaluate the following shark antibodies and their analogs for diagnostics, therapeutics and diagnostics with therapeutic (theranostic) applications.

### Generation of Shark Parent Antibody and Analogs

The sharks will be immunized with one or more antigens using the biomarkers associated with different diseases and/or organisms. Shark heavy chain only antibodies and their analogs can be generated by either by protease digestion, recombinant or chemical means. The analogs may be monovalent, divalent or multivalent (e.g., tri-, tetra-, penta-valent).

Production of Shark Heavy Chain Only Antibodies and their Analogs by Recombinant Means

Shark mRNA encoding heavy chain only antibodies will be isolated from the sharks with or without being immunized with one or more antigens using the biomarkers associated with different diseases and/or organisms. Various analogs will be generated by recombinant means using the standard methodology known in the art. See, Sambrook et al., Molecular Cloning: A Laboratory Manual (2nd Ed.) (1989), Cold Spring Harbor Press, N.Y. Various methods of extraction are suitable for isolating the DNA or RNA. Suitable methods include phenol and chloroform extraction. See Maniatis et al., Molecular Cloning, A Laboratory Manual, 2d, Cold Spring Harbor Laboratory Press, page 16.54 (1989). Numerous commercial kits also yield suitable DNA and RNA including, but not limited to, QIAamp™ mini blood kit, Agencourt Genfind™, Roche Cobas® Roche MagNA Pure® or phenol: chloroform extraction using Eppendorf Phase Lock Gels®, and the NucliSens extraction kit (Bio\dot{m}erieux, Marcy l'Etoile, France). In other methods, mRNA may be extracted using MagNA Pure LC mRNA HS kit and Mag NA Pure LC Instrument (Roche Diagnostics Corporation, Roche Applied Science, Indianapolis, Ind.). Other published protocols and commercial kits are available including, for example, Qiagen products such as the QiaAmp DNA Blood MiniKit (Cat.#51104, Qiagen, Valencia, Calif.), the QiaAmp RNA Blood MiniKit (Cat.#52304, Qiagen, Valencia, Calif.); Promega products such as the Wizard Genomic DNA Kit (Cat.# A1620, Promega Corp. Madison, Wis.), Wizard SV Genomic DNA Kit (Cat.# A2360, Promega Corp. Madison, Wis.), the SV Total RNA Kit (Cat.# X3100, Promega Corp. Madison, Wis.), PolyATract System (Cat.# Z5420, Promega Corp. Madison, Wis.), or the PurYield RNA System (Cat.# Z3740, Promega Corp. Madison, Wis.).

Nucleic acid extracted can be amplified using nucleic acid amplification techniques well know in the art. Nucleic acid amplification can be linear or exponential. By way of example, but not by way of limitation these techniques can include the polymerase chain reaction (PCR) reverse transcriptase polymerase chain reaction (RT-PCR). Oligonucleotide primers for use in these methods can be designed according to general guidance well known in the art as described herein, as well as with specific requirements as described herein for each step of the particular methods described.

### Amplification of Shark IgNAR Gene

All or portions of IgNAR gene can be amplified using different combinations of forward and reverse primers. Sequences of exemplary forward and reverse primers to amplify all or portions of shark IgNAR gene are shown below:

| | |
|---|---|
| Forward Primers | |
| 5′-gcatgggtag accaaacaccaag-3′ | (SEQ ID NO: 18) |
| 5′-gcgtcctcagagagagtcccta-3′ | (SEQ ID NO: 19) |
| 5′-gagacggacgaatcactgaccatc-3′ | (SEQ ID NO: 20) |
| 5′-gggtagaccaaacaccaagaacagc-3′ | (SEQ ID NO: 21) |
| Reverse Primers | |
| 5′-gttctagccaataggaacgtatag-3′ | (SEQ ID NO: 22) |
| 5′-gtttgcacaagagagtagtctttac-3′ | (SEQ ID NO: 23) |
| 5′-cctaaattgtcacagcgaatcatg-3′ | (SEQ ID NO: 24) |
| 5′-gtgcagttccctagaagtcttg-3′ | (SEQ ID NO: 25) |

### Amplification of Shark VNAR-HR-CH1 Domains:

All or portions of VNAR-HR-CH1 region can be amplified using the various combinations of forward and reverse primers. Sequences of exemplary forward and reverse primers are shown below:

| | |
|---|---|
| Forward Primers | |
| 5′-gcatgggtag accaaacaccaag-3′ | (SEQ ID NO: 26) |
| 5′-gcgtcctcagagagagtcccta-3′ | (SEQ ID NO: 27) |
| 5′-gagacggacgaatcactgaccatc-3′ | (SEQ ID NO: 28) |
| 5′-gggtagaccaaacaccaagaacagc-3′ | (SEQ ID NO: 29) |
| Reverse Primers | |
| 5′-cacctcttccgacatgaggtcc-3′ | (SEQ ID NO: 30) |
| 5′-gattaaagaaaggaaaccaatgac-3′ | (SEQ ID NO: 31) |
| 5′-cagctacaaaagtaactcccac-3′ | (SEQ ID NO: 32) |
| 5′-gagagacaagaagtcaacgtctcat-3′ | (SEQ ID NO: 33) |

### Amplification of Shark VNAR-HR-CH1-CH2 Domains:

All or portions of VNAR-HR-CH1-CH2 region can be amplified using the various combinations of forward and reverse primers. Sequences of exemplary forward and reverse primers are shown below:

| | |
|---|---|
| Forward Primers | |
| 5′-gcatgggtag accaaacaccaag-3′ | (SEQ ID NO: 34) |
| 5′-gcgtcctcagagagagtcccta-3′ | (SEQ ID NO: 35) |
| 5′-gagacggacgaatcactgaccatc-3′ | (SEQ ID NO: 36) |
| 5′-gggtagaccaaacaccaagaacagc-3′ | (SEQ ID NO: 37) |
| Reverse Primers | |
| 5′-ggtgagacggtgagaaggtgcg-3′ | (SEQ ID NO: 38) |
| 5′-ggaagccaggaatggaaaggtag-3′ | (SEQ ID NO: 39) |
| 5′-ccaaaggtaggtaaaatcgacg-3′ | (SEQ ID NO: 40) |
| 5′-ggtctaaagaagttgactacctag-3′ | (SEQ ID NO: 41) |

### Amplification of Shark VNAR-HR Domains

All or portions of VNAR-HR region can be amplified using the various combinations of forward and reverse primers. Sequences of exemplary forward and reverse primers are shown below:

| | |
|---|---|
| Forward Primers | |
| 5′-gcatgggtag accaaacaccaag-3′ | (SEQ ID NO: 42) |
| 5′-gcgtcctcagagagagtcccta-3′ | (SEQ ID NO: 43) |
| 5′-gcatgggtag accaaacaccaag-3′ | (SEQ ID NO: 44) |
| Reverse Primers | |
| 5′-ccacctcttccgacatgaggtcc-3′ | (SEQ ID NO: 45) |
| 5′-ctcatttcatctgactactgacc-3′ | (SEQ ID NO: 46) |
| 5′-tcttccgaacatgaggtccaaagtg-3′ | (SEQ ID NO: 47) |

### Developing Analogs of Single Domain Heavy-Chain Antibodies by Chemical Means

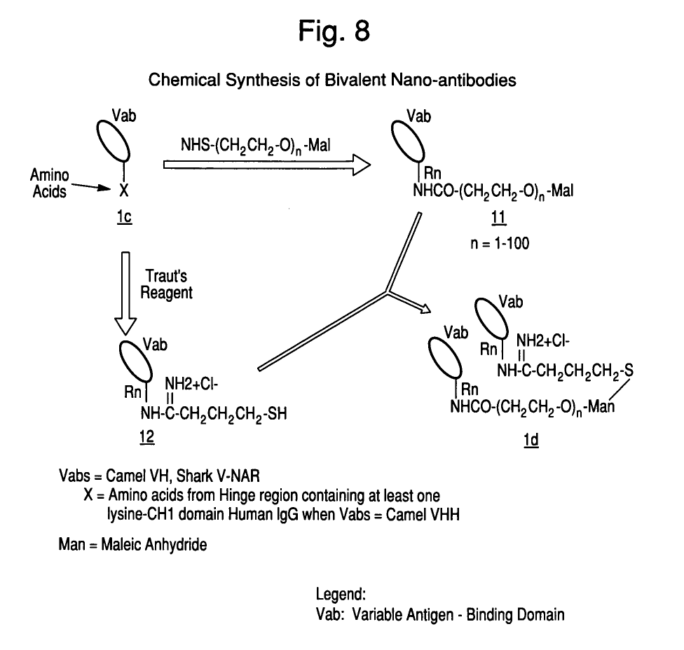

Analogs may also be generated by chemical and enzymatic treatment of camelid heavy chain only antibodies and analogs thereof. The analogs may be monovalent, divalent or multivalent (e.g., tri-, tetra-, penta-valent). Series of novel analogs of heavy-chain antibodies may be generated by chemical means or enzymatic means. Exemplary methods are disclosed in FIGS. 8-14 and 17-19.

In one embodiment, camelid and shark heavy chain only antibodies (structures 1 and 2 respectively) will be purified using standard methods of purifying antibodies. Exemplary method of antibody purification is shown in Example 12. Camelid and shark heavy chain only antibodies will then be used to develop several analogs by chemical techniques.

Protease digestion: In one embodiment, camelid and shark heavy chain only antibodies structures 1 and 2 will be treated with Tris-carboxyethyl phosphine (TCEP) to generate single chain analog of structure 1a and 52 respectively. Alternatively, camelid and shark heavy chain only antibody can be treated with reducing agents such as beta mercaptoethanol or dithiothreitol. In another embodiment, the single chain analog (structures 1a and 52) can be treated with proteolytic enzymes such as pepsin, trypsin or papain to generate analogs of smaller size. For example, single chain analog can be treated with pepsin under controlled condition to generate an analog of structure of structure 1b and 55. In another embodiment, the pepsin digests (structures 1b and 55) can be further subjected to proteolytic treatment, such as with trypsin to generate a smaller fragment such as structures 1c and 53. In another embodiment, structure 53 can be further fragment by protease digest such as pepsin, trypsin, papain under controlled condition to generate shark heavy chain only antibody analog comprising all or portions of NAR and all or portions of HR domains, such as structure 72.

Chemical methods: Commercial kits are available for protein conjugation, protein crosslinking. Crosslinking can be done with or without spacers of various lengths. Crosslinking can be done for example, between two primary amines using commercially available reagents (e.g., BS(PEG)9, BS(PEG)5, EGS, BSOCOES, DSP, DSG, from Thermo Scientific, Rockford, Ill.), between a primary amine and a sulfhydryl group using commercially available reagents (e.g., SM(PEG)24, SM(PEG)12, LC-SMCC, Sulfo-SMCC, Sulfo-LC-SPDP, Sulfo-EMCS, SMCC, from Thermo Scientific, Rockford, Ill.). Proteins can be derivatized to generate new functional groups. For example, N-hydroxysulfosuccinimide (Sulfo-NHS) and its uncharged analog N-hydroxysuccinimide (NHS) are used to convert carboxyl groups to amine-reactive Sulfo-NHS esters. Traut's reagent can be used to generate a sulfhydryl group. Proteins can be pegylated using commercially available reagents. For example, SM(PEG)n, BS(PEG)9, BS(PEG)5 from Thermo Scientific, Rockford, Ill.

### Genetically Modifying Host Cells by Introducing Recombinant Nucleic Acid

The recombinant nucleic acid (e.g., cDNA or genomic DNA) encoding at least a portion of a polypeptide may be introduced into host cells thereby genetically modifying the host cell. Host cells may be used for cloning and/or for expression of the recombinant nucleic acid. Host cells can be prokaryotic, for example bacteria. Host cell can be also be eukaryotic which includes but not limited to yeast, fungal cell, insect cell, plant cell and animal cell. In one embodiment, the host cell can be a mammalian cell. In another embodiment host cell can be human cells. Host cells may comprise wild-type genetic information. The genetic information of the host cells may be altered on purpose to allow it to be a permissive host for the recombinant DNA. Examples of such alterations include mutations, partial or total deletion of certain genes, or introduction of non-host nucleic acid into the host cell. Host cells may also comprise mutations which are not introduced on purpose.

Several methods are known in the art to introduce recombinant DNA in bacterial cells that include but are not limited to transformation, transduction, and electroporation, see Sambrook, et al., Molecular Cloning: A Laboratory Manual (1989), Second Edition, Cold Spring Harbor Press, Plainview, N.Y. Non-limiting examples of commercial kits and bacterial host cells for transformation include NovaBlue Singles™ (EMD Chemicals Inc, NJ, USA), Max Efficiency® DH5α™, One Shot® BL21 (DE3) E. coli cells, One Shot® BL21 (DE3) pLys E. coli cells (Invitrogen Corp., Carlsbad, Calif., USA), XL1-Blue competent cells (Stratagene, CA, USA). Non limiting examples of commercial kits and bacterial host cells for electroporation include Zappers™ electrocompetent cells (EMD Chemicals Inc, NJ, USA), XL1-Blue Electroporation-competent cells (Stratagene, CA, USA), ElectroMAX™ A. tumefaciens LBA4404 Cells (Invitrogen Corp., Carlsbad, Calif., USA).

Several methods are known in the art to introduce recombinant nucleic acid in eukaryotic cells. Exemplary methods include transfection, electroporation, liposome mediated delivery of nucleic acid, microinjection into to the host cell, see Sambrook, et al., Molecular Cloning: A Laboratory Manual (1989), Second Edition, Cold Spring Harbor Press, Plainview, N.Y. Non-limiting examples of commercial kits and reagents for transfection of recombinant nucleic acid to eukaryotic cell include Lipofectamine™ 2000, Optifect™ Reagent, Calcium Phosphate Transfection Kit (Invitrogen Corp., Carlsbad, Calif., USA), GeneJammer® Transfection Reagent, LipoTAXI® Trasfection Reagent (Stratagene, CA, USA). Alternatively, recombinant nucleic acid may be introduced into insect cells (e.g. sf9, sf21, High Five™) by using baculo viral vectors.

Selected positive clones will be used to subclone in both eukaryotic and prokaryotic expression vectors used standard methods known in the art. Exemplary eukaryotic expression vectors include expression vector pCMV SPORT (Invitrogen, Carsbad, Calif.), pExchange, pCMV-Script, pCMV-Tag (Stratagene). Exemplary prokaryotic expression vectors include pET expression vectors (Novagen®).

### Protein Purification:

Proteins from samples can be isolated using techniques that are well-known to those of skill in the art. The protein isolation methods employed can, e.g., be including, but not limited to, e.g., those described in Harlow & Lane, Antibodies: A Laboratory Manual (Cold Spring Harbor Laboratory Press, Cold Spring Harbor, N.Y., 1988), U.S. Pat. Nos. 6,005,079, 5,759,808. In some embodiments, an antigen protein is extracted from the acellular body fluid sample. Plasma purification methods are known in the art as such. See e.g., Cohn, E. J., et al., Am. Chem. Soc., 62:3396-3400.(1940); Cohn, E. J., et al., J. Am. Chem. Soc., 72:465-474 (1950); Pennell, R. B., Fractionation and isolation of purified components by precipitation methods, pp. 9-50. In The Plasma Proteins, Vol. 1, F. W. Putman (ed.). Academic Press, New York (1960); and U.S. Pat. No. 5,817,765. In brief, total IgGs will be precipitated from camelid or shark serum using 5M ammonium sulfate. The precipitated mixture of IgGs will be size fractionated on a long (200 cm×1 cm) Sephadex G-50 or G-200 column to fractionate 90 K Da camel mini-antibody from 150 K Da conventional IgG. Any residual contamination of conventional IgG can be removed by magnetic beads coated with protein-G. If needed. Final purification can be done by affinity purification. The same protocol for isolation and purification of shark mini-antibodies will be used after treating the precipitated total shark IgGs with TCEP.

### Characterization of Camelid and Shark Heavy-Chain Only Antibodies and Analogs

Characterization of the camelid and shark heavy-chain only antibodies and analogs will be done with ELISA, Affinity Determination and Western Blot assays:

ELISA Assay: The specificity and reactivity of the camelid and shark heavy-chain only antibodies and analogs towards the natural and synthetic antigens will be determined using ELISA. ELISA assays will be performed with pre-made reagents purchased from vendors such as Pierce, Sigma, etc., following vendor protocol, using appropriate negative and positive controls will be also be used.

Affinity Determination: 96-Microwell plates will be coated with heavy-chain only antibodies and their analogs, and conventional mouse monoclonal antibody (as a control) in different concentrations (for example, 1, 20, 40. 80, 160 ng/ul), and will be blocked with BSA overnight. The blocked antibody will be reacted with the peptide antigen conjugated to HRP for 1-2 hours at 37° C. After thoroughly washing the plate with a plate washer with 1×PBS containing 0.5% NP-40, enzyme substrate will be added and the plate incubated at RT for 1-2 hours. The optical density of the color generated will be read at an appropriate wave length. The affinity will then be calculated using the method of Beatty et al [J. Immunol. Methods, 100, 173 (1987)].

Western Blot Assay: Western blot will be performed using methods known in the art. See, Harlow & Lane, Antibodies: A Laboratory Manual (Cold Spring Harbor Laboratory Press, Cold Spring Harbor, N.Y., 1988); Sambrook et al., Molecular Cloning: A Laboratory Manual (2nd Ed.) (1989), Cold Spring Harbor Press, N.Y.

Immobilization of Antibodies onto Solid Supports:

The antibody or a sample may be immobilized on a carrier or solid support by covalent or non-covalent means. Immobilization of the antibodies or its analogs to the solid support may be done prior to, subsequent to, or simultaneously with binding to an antigen. Well-known supports or carriers include, but are not limited to, e.g., glass, microchannels, microfluidic device, polystyrene, polypropylene, polyethylene, latex, dextran, nylon, amylases, natural and modified celluloses, polyacrylamides, gabbros, nanoparticles, gold, and magnetite. The support material may have any possible configuration including spherical (e.g. bead), cylindrical (e.g. inside surface of a test tube or well, or the external surface of a rod), or flat (e.g. sheet, test strip).

In preferred embodiments, the solid surface is a bead. In some embodiments, beads or microparticles are substantially the same size. In other embodiments, beads or microparticles are of one or more sizes. In one embodiment, the beads or microparticles may be magnetic. In some embodiments, the preferred surface is microchannels made of glass or any other suitable matrix. These beads or microparticles may be composed of, for example, polystyrene, gold or latex. Beads or microparticles may be approximately 0.1 μm-10 μm in diameter or may be as large as 50 μm-100 μm in diameter, however, smaller and larger bead sizes are possible.

In one embodiment, the solid surface is a streptavidin coated bead. Streptavidin coated beads are available commercially e.g., from Bang laboratories (Catalog No. 214, 217), EMD Biosciences (Catalog No. 70716-3, 70716-4), Dynal beads from Invitrogen Corporation (Catalog No. 658-01D, 602-10).

In some embodiments, the solid surfaces may have functional groups capable of covalently linking the antibodies or its analogs directly or indirectly through chemical linkers. Examples of functional groups include but not limited to poly L-lysine, aminosilane, epoxysilane, aldehydes, amino groups, epoxy groups, cyano groups, ethylenic groups, hydroxyl groups, thiol groups.

A preferred method of non-covalently immobilizing antibodies or their analogs to the solid surface is via a “binding pair,” which refers herein to two molecules which form a complex through a specific interaction. Thus, the antibodies or its analogs can be captured on the solid support through an interaction between one member of the binding pair linked to the antibodies or its analogs and the other member of the binding pair coupled to the solid support.

In a preferred embodiment, the binding pair is biotin and avidin, or variants of avidin such as streptavidin, NeutrAvidin™. The solid surface may comprises streptavidin or its variants and the antibodies or its analogs may be modified to consist of biotin. Methods for biotinylating antibodies or its analogs are known in the art (e.g. through primary amine by NHS-PEO12-Biotin, NHS-LC-LC-Biotin, NHS-SS-PEO4-Biotin from Pierce Chemical Co.; through sulfhydryl group by Maleimide-PEO11-Biotin, Biotin-BMCC Sulfhydryl, Iodacetyl-PEO2-Biotin).

In other embodiments, the binding pair consists of a ligand-receptor, a hormone-receptor, an antigen-antibody. Examples of such binding pair include but are not limited to digoxigenin and anti-digoxigenin antibody; 6-(2,4-dinitrophenyl) aminohexanoic acid and anti-dinitrophenyl antibody; 5-Bromo-dUTP (BrdUTP) and anti-BrdUTP antibody; N-acetyl 2-aminofluorene (AAF) and anti-AAF antibody.

In another embodiment, the antibodies or their analogs may be anchored to the solid support covalently though chemical coupling using chemical linkers. If covalent bonding between the antibodies or their analogs and the surface is desired, the solid surface will usually be functional or be capable of being functionalized. Examples of functional groups used for linking include but are not limited to carboxylic acids, aldehydes, amino groups, cyano groups, ethylenic groups, hydroxyl groups, thiol groups. In one embodiment, the antibodies and their analogs can be covalently attached to the solid surface derivatized with primary amines through the sulfhydryl group using Sulfo-SMCC using manufacturer's protocol (Pierce Chemical Co.). Alternatively, sulfhydryl group can be introduced into the antibodies and their analogs using Traut's reagent or SATA (Pierce Chemical Co.) and such sulfhydryl group can be used to covalently link with the amine on the solid surface.

In some embodiments, the solid support may be coated with epoxy group, amino group, mercapto group, polylysine. Coated solid supports are available commercially e.g., beads coated with functional groups are available from Invitrogen Corporation, BD Biosciences; glass slides coated with functional groups are available from Pierce, Asper Biotech, Full Moon Biosystems, ThermoFisher Inc.

### Detectable Labels:

Antibodies may be detectably labeled by methods known in the art. Labels include, radioisotopes, enzymes (e.g., peroxidase, alkaline phosphatase, beta-galactosidase, luciferase, alkaline phosphatase, acetylcholinesterase and glucose oxidase), enzyme substrates, luminescent substances (e.g., luminol), fluorescent substances (e.g., FITC, rhodamine, lanthanide phosphors), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags) and colored substances. In binding these labeling agents to the antibody, the maleimide method (Kitagawa, T., et al., J. Biochem., 79:233-236 (1976)), the activated biotin method (Hofmann, K., et al., J. Am. Chem. Soc., 100:3585 (1978)) or the hydrophobic bond method, for instance, can be used.

Detectable labels include but are not limited to fluorophores, isotopes (e.g. ³²P, ³³P, ³⁵S, ³H, ¹⁴C, ¹²⁵I, ¹³¹I), electron-dense reagents (e.g., gold, silver), nanoparticles, enzymes (e.g., peroxidase, alkaline phosphatase, beta-galactosidase, luciferase, alkaline phosphatase, acetylcholinesterase and glucose oxidase), enzyme substrates, luminescent substances (e.g., luminol), chemiluminiscent compound, colorimetric labels (e.g., colloidal gold), magnetic labels (e.g., Dynabeads™), biotin, digoxigenin, haptens, proteins for which antisera or monoclonal antibodies are available, ligands, hormones, oligonucleotides capable of forming a complex with the corresponding oligonucleotide complement.

In a preferred embodiment, the detectable label is a fluorophore. The term “fluorophore” as used herein refers to a molecule that absorbs light at a particular wavelength (excitation frequency), and subsequently emits light of a different, typically longer, wavelength (emission frequency) in response. In one embodiment, the detectable label is a donor fluorophore in close proximity of a quencher moiety.

Suitable fluorescent moieties include but are not limited to the following fluorophores working individually or in combination:

4-acetamido-4′-isothiocyanatostilbene-2,2′ disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-di isothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY®21; QSY®35 (Molecular Probes); Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC).

### Medical Applications:

### Development of Nano-Biomedical Technology Platforms Using Heavy-Chain Antibodies and Analogs:

Novel analogs of single-domain heavy-chain only camelid and shark antibodies can be used for developing Nano-biomedical Technology Platforms to overcome problems of the conventional antibodies: i) the conventional antibodies neither have the specificity, nor sensitivity, nor thermal and chemical stability that allows the use of stringent assay development conditions to optimize detection sensitivity and specificity; ii) they are unable to cross cell membrane and blood brain barrier (BBB); iii) they are immunogenic; and iv) high toxicity due to cross-reactivity. The shark and camelid antibodies, particularly, their analogs do not have the shortcomings of conventional mAbs. First, these antibodies are small enough to cross cell and BBB to diagnose and treat most diseases that so far have been impossible to diagnose and treat without invasive and risky procedures. Second, they are highly specific and have very little to none cross-reactivity. Third, these antibodies have extremely low immunogenicity, and can be further humanized to take care of any residual toxicity.

Biodistribution studies in solid tumors have shown that whole IgG molecules are too large to enter the cell while the smaller antigen-binding camel Vab fragments (MW ˜12-17 KDa) rapidly clears from the body [Nature Medicine, 9, 129 (2003)]. So the large molecular weight (˜150 KDa) and small molecular weight (˜15 KDa) biological molecules are not suitable for treating diseases of the brain and cancer. The best tumor targeting reagents comprise an intermediate-sized multivalent molecules (MW˜55 KDa), providing rapid tissue penetration, high target retention, and rapid blood clearance [Nature Medicine, 9, 129 (2003)]. That is why, most of the analogs of shark and camelid heavy chain antibodies of this application have been carefully designed so as to have molecular weight in the range of 30 to 60 KDa for optimal biodistribution and retention, though analogs with molecular weight between 60 to 90 KDa will also be studied to explore their hitherto unknown and unstudied properties to cross cell membrane and BBB.

Applications of Shark and Camelid Heavy Chain Only Antibodies and their Analogs

Shark and camelid heavy chain only antibodies and their analogs can be used in diagnostics, therapy and simultaneous diagnosis and treatment. Exemplary areas of applications include:

### 1. In-Vitro Diagnostics:

a. Immunodiagnostics of human diseases

b. DNA-Probes Based Diagnostics of human diseases.

### 2. In-vivo Diagnostics

a. Neuroimaging and whole body scan

### 3. Rare Cell Capture

a. Circulating Tumor Cells (CTCs) for interrogation

b. Circulating fetal cells for non-invasive prenatal diagnosis of genetic disorders

### 4. Integrated Diagnostics and Therapeutics (Theranostics)

a. Cancer

b. Neurodegenerative diseases

c Brain cancer and brain disorders

d. Diseases of immune disorder

e. Infectious diseases

f. Metabolic diseases

g Biological warfare, Anthrax, SARS etc.

h. Cardiac diseases

### 5. Drug Transport

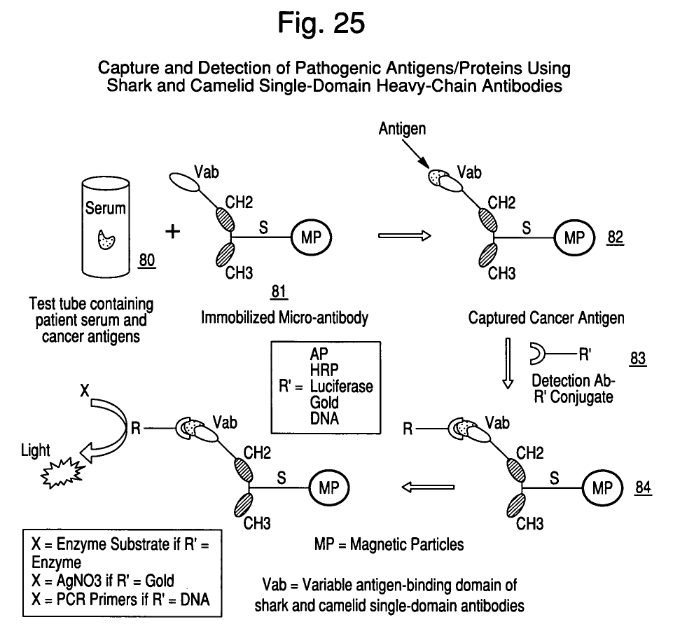

Exemplary applications of the shark and camelid heavy chain only antibodies and their analogs in diagnosing, treating, and simultaneous diagnosis and treatment are shown in Examples 18-22 and FIG. 25-29.

### Neuro-Imaging and Whole Body Scan

Since shark and camelid antibodies are known to cross cell wall and blood brain barrier, shark and camelid heavy chain only antibodies and their analogs against the biomarkers of brain diseases and the cytoplasmic markers of cancer will be useful in scanning the whole body for early detection of cancer, Alzheimer's disease, Parkinson's disease and other brain diseases. For example, breast and lung cancers can be screened and diagnosed with a mixture of nano-antibodies against HER-2, p53, EGFR, and Ras. Brain cancer and Alzheimer's diseases can be detected and treated, in principle, with BBB permeable mixture of nano-antibodies against TEM1 (tumor endothelial marker-1), amyloid-β42, Tau protein, beta- and gamma-secretases.

Neuroimaging of brain diseases will be done with detectably labeled (e.g., radiolabel) antibodies and their analogs which will be administered intravenously and detected using appropriate methodology for example, the brain scanned under PET scanner after a short time thereafter.

### Capture of Circulating Fetal Cells for Non-Invasive Prenatal Diagnosis of Genetic Disorders

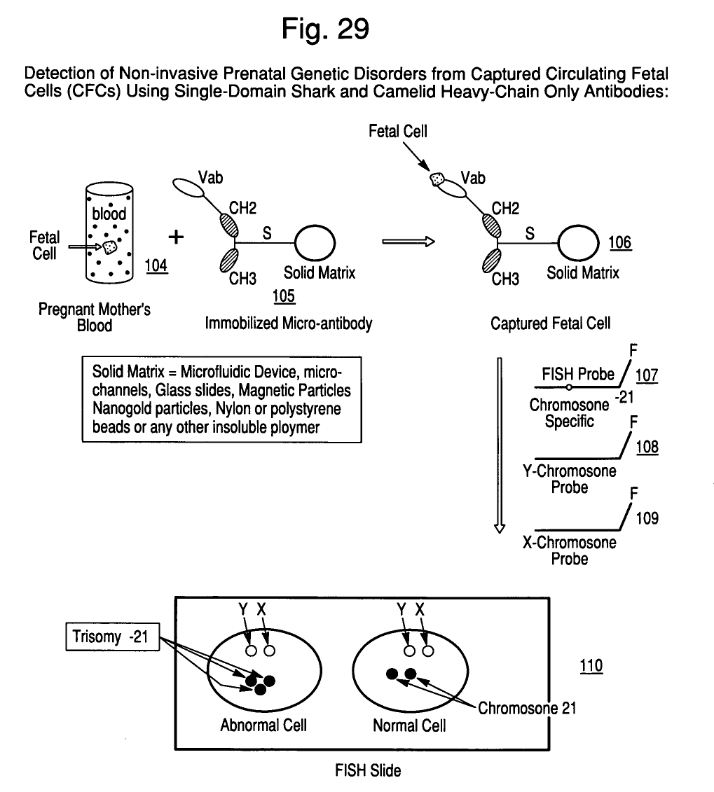

In one embodiment, fetal cells from the blood of pregnant women will be captured with shark and camelid heavy chain only antibodies and their analogs against the fetal cell surface antigens such as, CD71, glycophorin-A (GPA), CD133, CD34, HLA-G233, and Trop-1, which is/are bound to solid matrixes such as micro-channels and beads. The red blood cells will be lysed using commercial RBC lysis buffer. The cells will then be pelleted and passed through micro-fluidic device coated with a mixture of the above antibodies. The captured cells will be analyzed by FISH probes for chromosomes 21, 13 and 18. Exemplary schematics of the capture of circulating fetal cells by shark and camelid heavy chain only antibodies and their analogs is shown in FIG. 29.

### Detecting Cell-Free Tumor DNA:

In one embodiment, shark and camelid heavy chain only antibodies and their analogs can be used in isolation of cell free nucleic acids circulating in bodily fluids, blood, marrow, urine, saliva, CSF and cervical mucus. It is known that cell free DNA is elevated in the blood of cancer patients [T. L. Wu, et al., Clin. Chim. Acta., 321, 77 (2002)]. Though the cell free DNA in blood is known for many years, its clinical utility has not been established in spite of the fact that cell-free DNA has exhibited all the characteristics as the tumor DNA [P. Anker, et al., Cancer Metastasis Rev., 18, 65 (1999)]. Accordingly, shark and camelid heavy chain only antibodies and their analogs can be used, for example tumor biomarkers. against HER-2 (implicated in breast cancer), TMRESS2-ERG gene (implicated in prostate cancer), K-ras (pancreatic carcinoma) will provide a non-invasive method for diagnosing these diseases without undergoing invasive painful biopsies.

Shark and Camelid Heavy Chain Only Antibodies and their Analogs in Diagnostics and Therapy

Shark and camelid heavy chain only antibodies and their analogs have tremendous potential to overcome the shortcomings of classical antibodies such as lack of specificity, toxicity, immunogenicity and inability to cross cell membrane and BBB. Their smaller size, unusual stability to low pH and high temperatures, higher binding affinity, very little to none cross-reactivity but above all, their ability to cross cell-membrane and BBB make these molecules the most versatile molecules of this century for medical diagnostics and therapeutics. Generation of shark and camelid heavy chain only antibodies and their analogs against all known biomarkers and therapeutic targets will dramatically improve the medical benefits of antibodies. Their ability to enter the cell and cross BBB provides for the first time ever an opportunity to develop simultaneous diagnosis and treatment (theranostics) of human diseases.

### Diagnostic Markers:

Camelid or shark can be immunized with the pathogenic proteins associated with the disease discussed above. Heavy chain-only antibodies and their analogs that specifically bind to these biomarkers can be made from the parent single-domain heavy-chain only antibodies using various methods such as, protease digestion, recombinant DNA technology and chemical means as described above. Such antibodies and their analogs can be used in in-vitro diagnostics of human diseases, capture of circulating fetal cells for prenatal diagnosis, and capture of tumor cells for studying gene expression of key proteins pre- and post treatment.

### Prostate Cancer:

Prostate cancer is a disease in which cancer develops in the prostate, a gland in the male reproductive system. Rates of prostate cancer vary widely across the world. According to the American Cancer Society, prostate cancer is least common among Asian men and most common among black men, with figures for European men in-between. However, these high rates may be affected by increasing rates of detection. Prostate cancer develops most frequently in men over fifty. It is the most common type of cancer in men in the United States, where it is responsible for more male deaths than any other cancer, except lung cancer. However, many men who develop prostate cancer never have symptoms, undergo no therapy, and eventually die of other causes.

Camelid or shark can be immunized with the biomarkers associated with prostate cancer. Heavy chain-only antibodies and their analogs that specifically bind to these biomarkers can be made using various methods such as, protease digestion, recombinant DNA technology and chemical means as described above. Heavy chain-only antibodies and their analogs that specifically bind to these biomarkers can be used in diagnosis, prognosis and/or treatment of prostate cancer. Exemplary prostate cancer biomarkers are shown in Table 2.

TABLE 2
Biomarkers s Associated with Prostate Cancer

| | PubMed Unique Identifier |
|---|---|
| | or |
| Biomarker | Reference |
| 34betaE12 | 12558749 |
| Alkaline Phosphatase | 9096267 |
| AMACR (alpha-methylacyl-CoA | 15330799, 15323145, |
| racemase) | 11956072 |
| TMPRSS2-ERG Fusion Protein | ACA81385 |
| Apolipoprotein A-II | 15709174 |
| Apolipoprotein-D | 14716735 |
| beta hCG | 8655711 |
| Bcl-2 | 17062688, 12821128, |
| | 9836559 |
| Caveolin-1 | 10582690 |
| Chromogranin-A (CgA) | 16450720, 11792908, |
| | 8693652 |
| DPIV (Dipeptidylpeptidase IV) | 16094078 |
| E-Cadherin | 7506464 |
| EMBP (Estramustine-binding protein) | 8653325 |
| Endoglin (CD105) | 11987155 |
| EPCA-2 | 17445657 |
| Fatty Acid Synthase (FAS) | 11176534 |
| Gamma-Sm (gamma-Seminoprotein) | 2467541 |
| Glyoxalase I | 9915484 |
| HGF (Hepatocyte growth factor) | 16508682 |
| hK2 | 8950360 |
| ICTP | 11490307, 10221259 |
| IgBF (immunoglobulin gamma binding | 7509484 |
| factor) | |
| IGF-I | 12873996 |
| IGFBP-3 (insulin-like growth factor | 12497585 |
| binding protein-3) | |
| ILK | 15870868 |
| Ki-67 | 8709308 |
| LDH (lactate dehydrogenase) | 3956838 |
| Mcm7 | 15452160 |
| MUC6 | 12657938 |
| Nav1.7 | 16088330 |
| NFkapp-HCAb | 16980232 |
| p53 | 9815924 |
| P504S | 12218573, 11684956 |
| PCA-1 | 16033822 |
| PCADM-1 | 15073124 |
| PCNA (Proliferating cell nuclear antigen) | 16834658 |
| PhIP | 16889804 |
| PICP and ICTP | 9351545 |
| Pin1 | 14559810 |
| PSA (prostate-specific antigen) | 12581210, 12024909, |
| | 7508631, 1279493, 2442609 |
| PSMA (Prostate-specific membrane | 15837926 |
| antigen) | |
| PSP94 (prostate secretory protein of 94 | 16278416 |
| amino acids) | |
| PTEN | 17163422 |
| Reg IV | 15788672 |
| Reelin | 17277764 |
| RKIP | 16175585 |
| RM2 antigen | 15704108 |
| RPL19 | 16609016 |
| S100A6 | 15280928 |
| SAP (skeletal alkaline phosphatase) | 9736988 |
| SIM2 | 17289882 |
| Telomerase | 15053304 |
| TRAP 5b (Tartrate-resistant acid | 15514730 |
| phosphatase 5b) | |
| Trisomy 7 | 7507696 |
| TRPV6 | 14586412 |
| Urinary tissue factor (UTF) | 7686076 |
| Human Glandular Kallikrein 2 | Prostate Cancer and Prostatic |
| | Diseases, 11, 112 (2008) |

### Breast Cancer:

Breast cancer is a cancer of the breast tissue. Worldwide, it is the most common form of cancer in females—affecting, at some time in their lives, approximately 192,000 new cases of breast cancer will be diagnosed in the US in 2009, and estimated 41,000 women will lose their lives to the disease this year. According to the United Nations World Health Organization, it is the leading cause of cancer deaths among women in the US and worldwide. Because the breast is composed of identical tissues in males and females, breast cancer also occurs in males, though it is far less common.

Camelid or shark can be immunized with the biomarkers associated with breast cancer. Heavy chain-only antibodies and their analogs that specifically bind to these biomarkers can be made using various methods such as, protease digestion, recombinant DNA technology and chemical means as described above. Heavy chain-only antibodies and their analogs that specifically bind to these biomarkers can be used in diagnosis, prognosis and/or treatment of breast cancer. Exemplary breast cancer biomarkers are shown below:

PIP: highly expressed for breast cancer. It has been identified in most breast cancer biopsies.
SCGB2A1: found in breast tumors.
SCGB1D2: highly expressed in breast tumor.
SBEM protein: expressed in >90% of invasive ductal carcinoma.
ESR1: is expressed in about 67% of all breast cancers and thus is known as the main discriminator in breast tumor classification. ESR1 is the main mediator of endocrine therapy, Tamoxifen, and its detection in breast tumors is thus of considerable clinical significance.
NKRD30A, c-B726P, NY-BR-1.
6-PGDH-HCAb (6-phosphogluconate dehydrogenase)
A better prognostic indicator in primary breast cancer than estrogen receptor status. PMID: 3797962
   17HSD-HCAb (17beta-hydroxysteroid dehydrogenase type 1)
An independent prognostic marker in breast cancer. PMID: 15492288

### 17HSD14-HCAb

A prognostic marker in estrogen receptor-positive breast cancer. PMID: 17145895

### 44-3A6-HCAb

May serve as an important marker in the differentiation of normal breast epithelium into an atypical or malignant lesion. PMID: 1660186
   aHIF-HCAb
In our series of breast cancer patients, aHIF, and not HIF-1alpha transcript, is a marker of poor prognosis. PMID: 14580258

### AP-HCAb for Alkaline Phosphatase (ALP)

Skeletal ALP could represent a valid marker for bone metastases in association with mucinous markers in the follow-up of patients operated for breast cancer. PMID: 7629426

### BRCA1-HCAb

A marker for inherited breast and ovarian cancers.

### BRCA2-HCAb

A marker for inherited breast and ovarian cancers.

### Alpha-Lactalbumin-HCAb

Serum levels of alpha-lactalbumin may be useful as a marker for monitoring breast cancer. PMID: 2337516
   AMAS-HCAb (anti-malignin antibody in serum)
More sensitive (97%) in detecting breast cancer than CEA (0%), CA 15-3 (10%), CA 19-5 (5%) or CA 125 (16%) in the same patients. PMID: 10680591

### AR-HCAb for Androgen Receptor (AR)

AR immunohistochemistry could serve as a marker to increase sensitivity for identifying breast cancer in skin metastasis of unknown primary sites. PMID: 10697267

### ANX7-HCAb for ANX7

Has prognostic value for predicting survival of breast cancer patients, is a bio-marker in prostate and breast cancer progression. PMID: 11673658
   Bcl-2-HCAb for detecting Bcl-2.
An independent predictor of breast cancer outcome and seems to be useful as a prognostic adjunct to the NPI, particularly in the first 5 years after diagnosis. PMID: 16638854
   Beta 1-6 branched oligosaccharides-HCAb
A marker of tumor progression in human breast and colon neoplasia. PMID: 1985789

### Beta-Catenin

Can be involved in breast cancer formation and/or progression and may serve as a target for breast cancer therapy. PMID: 10759547

### BNIP3-HCAb for BNIP

A progression marker in primary human breast cancer; opposing functions in in-situ versus invasive cancer. PMID: 17255267
   CA IX-HCAb for detecting CA1X
A marker of poor prognosis in premenopausal breast cancer patients and it is an independent predictor of survival in patients with one to three positive lymph nodes. PMID: 17085655

### CAXII-HCAb for Carbonic Anhydrase XII (CA-12)

A marker of good prognosis in invasive breast carcinoma. PMID: 12671706

### CA 15.3-HHCAb for CA15-3

Can predict survival in primary breast cancer. PMID: 12452445
Most widely used as a serum tumor marker in follow-up and detection of breast cancer recurrence: PMID: 11192831
Highly useful in the diagnosis, differential diagnosis, and monitoring of metastases and recurrences of breast cancer, and is superior to CEA. PMID: 10920962
A tumor marker associated with mammary tumors. PMID: 1563679
Significantly better than CEA in the detection of breast cancer metastases. PMID: 2065278
May thus be the first independent prognostic serum marker in breast cancer. PMID: 11192829

### CA27.29-HHCAb for CA27.29

Suitable for routine use in the management of patients with breast cancer. PMID: 11239757

### CA549-HHCAb for CA549

A new tumor marker for breast cancer. PMID: 8479100
A marker for breast cancer. PMID: 8160638
   CaR-HCAb for calcium-sensing receptor
Expression is common in a selected group of patients with advanced primary breast cancers. PMID: 16564154
   Carcinoembryonic antigen-HCAb for CEA
CEA monitoring should be considered an expensive and inefficient method of follow-up evaluation for breast cancer patients, and it provides no additional value when used in combination with CA 15.3. PMID: 11489813

### CD24-HCAb for CD24

CD24 expression in primary breast cancer as detected by immunohistochemistry might be a new marker for a more aggressive breast cancer biology. PMID: 14581365
A useful marker for human breast carcinoma and play a role in facilitating metastasis by the interaction between tumor cells and platelets or endothelial cells. PMID: 10465342

### CD44-HHCAb for CD44s

CD44s detection by immunohistochemistry is useful in distinguishing intraductal papillomas from papillary carcinomas of the breast. PMID: 10690183
   C-erbB2-HHCAb for C-erbB2 oncoprotein
Prognostic marker in breast cancer. PMID: 16286993
Overexpression of c-erbB2 in the primary tumor is an independent marker of relative resistance to first-line endocrine therapy in patients with advanced breast cancer. PMID: 10098763
   CK-BB-HCAb for Creatine kinase-BB
CSF activity of CK-BB appears to be a contribution in the diagnosis of MC secondary to breast cancer and seems superior to protein and LDH. PMID: 2632253

### Cyclin A-HCAb for Cyclin A

A good marker for tumor proliferation and prognosis in breast cancer. PMID: 16091759

### Cyclin E-HCAb for Cyclin E

In breast cancer, the alterations in cyclin E expression become progressively worse with increasing stage and grade of the tumor, suggesting its potential use as a new prognostic marker. PMID: 7903908
   CYFRA 21-1-HCAb for cytokeratin-19 fragments
A useful tumor marker for detecting disease relapse and assessing treatment efficacy in breast cancer. PMID: 15280913
   D2-40-HCAb for D2-40 antigen
Identifies lymphatic invasion in breast tumors and is a significant predictor of outcome in breast cancer. PMID: 17206106
   DAP-kinase-HCAb for death associated protein-kinase
Loss of DAP-kinase expression negatively correlates to survival and positively correlates to the probability of recurrence in a very significant manner. DAP-kinase thus constitutes a novel and independent prognosis marker for breast cancer. PMID: 15131053

### Endoglin-HCAb for CD105

A marker of breast carcinoma-induced neo-vascularization. PMID: 9858949
   ER-beta-HCAb for estrogen receptor beta
ER-alpha is more dysregulated in breast cancer, and thereby ER-beta is more tightly regulated in the tumor. PMID: 16289616
   ErbB-2-HCAb for C-erbB-2 oncogene protein
A prognostic marker of breast carcinoma, and serum ErbB-2 is a preoperative prognostic marker and may be useful for monitoring tumor recurrence of the breast. PMID: 17044019
The determination of ErbB-2 in tissue extracts of breast carcinoma may be useful for assessing c-erbB-2 protein expression in the primary tissue and indicates that serum ErbB-2 may be a sensitive marker for monitoring tumor relapse. PMID: 10956406
   Ets-1-HCAb for Ets-1 antigen
A strong, independent predictor of poor prognosis in breast cancer. PMID: 12466970
   EZH2-HCAb for enhancer of zeste homologue 2
A marker of aggressive breast cancer. PMID: 16489070
Significantly associated with increased tumor cell proliferation and is a marker of aggressive breast cancer. PMID: 16489070
A marker of aggressive breast cancer and promotes neoplastic transformation of breast epithelial cells. PMID: 14500907

### Ferritin-HCAb for Ferritin

A marker of therapeutic response in stage III and IV breast cancer. PMID: 2137790

### GATA3-HCAb for GATA3

May be the basis for a new clinically applicable test to predict tumor recurrence early in the progression of breast cancer. PMID: 16357129

### GCDFP-15-HCAb for GCDFP-15

A specific marker for breast cancer and is superior to ALA in this respect. PMID: 2542151

### GCDFP-24-HCAb for GCDFP-24

Could well be a good biochemical marker for monitoring the response to androgenic and antiestrogenic compounds in the therapy of advanced breast cancer. PMID: 2351114

### Glut-1-HCAb for Glut-1

TMA analysis for Glut-1 expression may be useful to predict disease free survival but it does not predict race specific recurrence. PMID: 16228617
   GST-HCAb for glutathione S-transferase
Measurement of GST B1 or GST B2 in lung lavage fluid could be a useful aid in the diagnosis of lung malignancy. PMID: 2302756

### HER2/Neu-HCAb for HER2/Neu

A predictor of prognosis in breast cancer and a potential marker for selecting the optimal adjuvant chemotherapy. PMID: 16137437
An important independent prognostic factor in early stage breast cancer. PMID: 10066073
Coexistence of HER2 over-expression and p53 protein accumulation is a strong prognostic molecular marker in breast cancer. PMID: 14680497

### HSP70-HCAb for HSP70

nuclear share of hsp70 is associated with various biological characteristics of malignant breast tumors, while the occurrence of cytoplasmatic hsp70 influences OS and SR. PMID: 12820347
   HIF-HCAb for Hypoxia inducible factor-1alpha
a prognostic marker in premenopausal patients with intermediate to highly differentiated breast cancer but not a predictive marker for tamoxifen response. PMID: 16381002-hK-HCAb for Human kallikrein 10 (hK10)
A predictive marker for breast cancer. PMID: 16800732

### IGFBP2-HCAb for IGFBP-2

A marker for antiestrogen resistant breast cancer cell lines, although IGFBP-2 was not a major contributor to the resistant cell growth. PMID: 16893667

### KAI-HCAb for KAI-1

In addition to its role in human prostate, pancreatic and non-small cell lung cancer, KAI1 may also be a useful marker for staging human breast disease. PMID: 9570365

### Ki-67-HCAb for Ki-67

A prognostic marker in early breast cancer. PMID: 17453008
Tumor proliferative activity as evaluated by the monoclonal antibody Ki-67 seems to be an effective indicator of prognosis in breast cancer for DFS and OS. PMID: 8508358

### KL6-HCAb for KL-6

Serum KL-6 may be helpful for clinical use as a tumor marker for breast cancer, and it may play an important role, especially in the surveillance of disease relapse. PMID: 11051258

### KLK15-HCAb for KLK15

An independent and favorable prognostic marker for breast cancer. PMID: 12439720
   KPNA2-HCAb for KPNA2 (karyopherin alpha2)
A potential novel prognostic marker in breast cancer. PMID: 16818692

### Laminin-HCAB for Laminin

Serum determination of laminin could be a useful diagnostic tool in breast cancer and a valuable parameter in the prediction of metastasis. PMID: 10356660

### Leptin-HCAb for Leptin

A marker of breast cancer progression: possible role of obesity-related stimuli. PMID: 16533767

### LY6K-HCAb for LY-6K

Not only a target antigen for HNSCC but also a significant new molecular marker for diagnosis and gene therapy in patients with breast cancer. PMID: 17089039

### M3/M21-HCAb for M3/M21

The detection of breast cancer recurrence with CA 15-3 is improved by combination with M3/M21. PMID: 8920765 MAGEA-HCAb for MAGE-A (Melanoma antigen gene A).
gene expression may be used for the surveillance of circulating breast carcinoma cells after primary therapy by RT-nested PCR using MMRPs. PMID: 15937912
   MAM6-HCAb for MAM-6 antigen
A new serum marker for breast cancer monitoring. PMID: 3697998

### MAM-HCAb for Mammaglobin (MAM)

As measured by the ELISA, holds significant promise for breast cancer screening with the realistic potential to impact management of this disease. PMID: 16166429
hMAM mRNA detection by RT-PCR is a specific assay potentially suitable for identification of occult cancer cells in peripheral blood of BC patients. PMID: 16110760
MAM gene expression on leukapheresis products of high-risk breast cancer patients is an indicator of poor prognosis. PMID: 15447988
Detection of mammaglobin protein and mRNA in clinical samples may be a useful marker for primary, metastatic, and occult breast cancer. PMID: 11193781
RT-PCR using mammaglobin B gene could therefore be a useful tool for detection of micrometastases of breast cancer. PMID: 10674878
A novel marker of minimal residual disease in early stages breast cancer. PMID: 15254674
One of the first relatively mammary-specific and mammary-sensitive markers. Mammaglobin and BRST-2 appear to represent useful markers for breast cancer and should be used as a component of panels evaluating tumors of unknown primary sites. PMID: 14521461
   MCA-HCAb for MCA (mucinous carcinoma associated antigen)
A promising tumor marker in breast cancer. Especially high values may have diagnostic significance. PMID: 3178156
The new tumor marker antigen MCA reacts with breast cancer cells in paraffin sections. It might be used in identification of cancer cells in tissue sections. MCA can also be used as a weak indicator of aggressiveness of the tumor. PMID: 1695077

### Mcm-HCAb for Mcm-2

May be of utility as a prognostic marker to refine the prediction of outcome in breast cancer, for example when combined with parameters currently used in the NPI. PMID: 14645419

### MDM2-HCAb for MDM2

A negative prognostic marker in breast carcinoma. PMID: 16258514

### MGB1-HCAb for MGB1

Can serve as a differential molecular marker. In practice, prospective examination, using the nine cases with a history of breast cancer, confirmed the usefulness of MGB1 in differential diagnosis. PMID: 15096563

### MIB-HCAb for MIB-1

The growth fraction of a tumor as determined by the MIB-1 labelling index is an important prognostic factor in patients with primary breast cancer. PMID: 9716027

### MMP1 for MMP-1

A candidate marker that may be useful for identification of breast lesions that can develop into cancer. PMID: 15864312

### MMP2-HCAb for MMP-2

MMP-2 immunoreactive protein has been associated strongly with a shortened survival independent of major prognostic indicators in patients with primary breast carcinoma, increasing the risk of death 3.6-fold during the first 10 years of follow-up. PMID: 9740080

### MMP13-HCAb for MMP-13

Primarily expressed by myofibroblasts in human breast carcinoma and that expression in DCIS lesions often are associated with microinvasive events. PMID: 11585740
   MSA-HCAb for MSA (Mammary serum antigen).
Serum levels do not allow discriminating benign from malignant breast diseases and MSA is 2.5 to 3 times more sensitive for the prediction of early stages breast cancer compared to CA15-3, TPA and CEA. PMID: 9082702
MSA levels are elevated in patients with breast cancer and may provide a useful means of following the clinical course of patients with this disease. PMID: 3355770
MSA levels may therefore be of some use for the monitoring of breast cancer patients, and as a diagnostic aid to screen populations for breast cancer. PMID: 3609486

### NCCST-HCAb for NCC-ST-439

The NCC-ST-439 level, especially in combination with the CEA level, may be useful for the early detection and the monitoring of relapses in breast cancer patients. PMID: 2232168

### Nectin4-HCAb for Nectin-4

A 66-kDa adhesion molecule of the Nectin family, which is a valuable new histological and serological marker for breast carcinoma. PMID: 15784625
   Neu-HCAb for Neu (c-erbB-2)
A tumor marker in carcinoma of the female breast. PMID: 1981830
   NRP1-HCAb for NRP-1 (neuropilin-1)
A marker of axillary lymph node breast metastases. PMID: 10451484
May be a multiple function protein in human breast and may be involved in the induction of local invasiveness of neoplasia and angiogenesis and have direct relevance to the progression of breast cancer. PMID: 12216067

### P53-HCAb for p53

Immunohistochemically detected p53 protein accumulation was an independent marker of shortened survival and was seen more often in familial than in sporadic carcinomas. PMID: 1317462
p53 positive Bcl-2 negative phenotype is an independent marker of prognosis in breast cancer. PMID: 17187363
p53 expression status was a significant molecular marker as well as the response to first-line endocrine therapy for predicting TTEF in recurrent breast cancer with hormone-sensitive disease. PMID: 17180510
Mutant p53 protein in serum could be used as a molecular marker in human breast cancer. PMID: 16525651
Nuclear p53 protein expression may represent an adverse prognostic marker in inflammatory breast cancer (IBC) and may provide a valuable tool for selecting treatment for this aggressive disease. PMID: 15448010

### P63-HCAb for p63

As part of the diagnostic workup of challenging spindle cell tumors of the breast as a highly specific marker for metaplastic carcinomas. PMID: 15489655

### P120-HCAb for P120

A prognostic marker in node-negative breast cancer. PMID: 7940158

### Pepsinogen-HCAb for Pepsinogen C

A new prognostic factor for early recurrence and death in both node-positive and node-negative breast cancer. PMID: 7799043

### PINP-HCAb for PINP

Serum marker of metastatic spread to the bone in breast cancer patients. PMID: 16033050
   PKC-alfa-HCAb for PKC alpha (Protein Kinase C alpha)
A marker for antiestrogen resistance and as a promising therapeutic target in the treatment of tamoxifen resistant breast cancer. PMID: 17061041

### PA-HCAb for Plasminogen Activator

Can be used as an effective functional marker for hormone dependence in human breast cancer. PMID: 3082829
   GalNAC-HCAb for ppGalNAc-T6
Could be a specific marker applicable to the molecular diagnosis of breast cancer cells dissemination. PMID: 16596643
a new immunohistochemical breast cancer marker. PMID: 16260590
   pS2-HCAb and c-ER-HCAb for pS2 and ER antigens
Are useful tools for predicting tumor regression with neoadjuvant tamoxifen in post-menopausal breast carcinoma patients. PMID: 8855985
   PSA-HCAb for PSA antigen
Might be useful as a marker for a subset of breast cancers with better prognosis, which could respond to endocrine therapy, in correlation with other prognostic markers. PMID: 16575473

### PSE-HCAb for PSE (Prostate Specific Ets)

A potentially informative novel marker for detection of metastatic breast cancer in axillary lymph nodes, and should be included in any study that involves molecular profiling of breast cancer. PMID: 11953821
   PTA-HCAb for PTA (prothymosin alpha)
A potential prognostic marker for primary breast cancer. PMID: 10682670

### PTEN-HCAb for PTEN

Might play an important and major role in its HER2/PI3K/Akt-mediated antitumor effect, and could be a useful biomarker for predicting the efficacy of trastuzum-Ab in the treatment of breast cancer. PMID: 16404430
   PTTG-HCAb for PTTG (pituitary tumor-transforming gene)
Expression in primary tumors of the breast is a powerful tool for the assessment of potential tumor aggressiveness. PMID: 14759723
   RCP-HCAb for RCP (riboflavin carrier protein)
Measurement of circulatory RCP and the immunohistochemical staining pattern of RCP in biopsy specimens could be exploited as an additional marker in diagnosis/prognosis of breast cancer in women. PMID: 11494224

### SigmaS-HCAb for Sigma S

A measure of reactive sulfur groups of immunoglobulin G, is a sensitive tumor marker discriminating different stages of breast cancer. PMID: 1913474

### SNCG-HCAb for SNCG

Expected to be a useful marker for breast cancer progression and a potential target for breast cancer treatment. PMID: 16821081
   SNSE-HCAb for S-NSE (serum neuron-specific enolase)
May be a useful marker for monitoring treatment and predicting relapse in patients with Small cell lung cancer (SCLC). PMID: 2155054

### S14-HCAb for Spot 14 (S14)

A marker of aggressive breast cancer and a potential therapeutic target. PMID: 16809441
Identifies a subset of high-risk breast cancers that is not specified by analysis of sex steroid receptors, Her2/neu, or cyclin D1, and provides a molecular correlate to histologic features that predict recurrence. PMID: 16552628

### ST439-HCAb for ST-439

A useful tumor marker not only in monitoring the recurrence, but also in the diagnosis of primary breast cancer. PMID: 2069399

### STC-1 for STC-1 (Stanniocalcin 1)

Is proposed as a novel, specific, and clinically useful molecular marker for detecting occult breast cancer cells in the BM and blood. PMID: 12684415

### S-HCAb for Survivin

Might be used as a new marker to stratify breast cancer patients for more optimal treatment modalities, or it could be a promising new target for therapy. PMID: 15364883
   TAG12-HCAb for TAG12 (Tumor associated glycoprotein 12)
a new tumor marker in breast cancer. PMID: 11326666
   TEM8-HCAb for TEM-8 antigen
A useful marker for identifying tumor associated micro-vessels and that elevated levels are associated with disease progression, which may have some bearing on the prognostic outcome in breast cancer. PMID: 17016666

### T-HCAb for Thioesterase II

May be a useful serum marker for mammary cancer. PMID: 3524817

### Thmb-HCAb for Thrombomodulin (TM)

Might play an active role in cancer invasion and metastasis, and serve as a new prognostic factor in invasive breast cancer. PMID: 9216709
   TIMP-1 (tissue inhibitor of metalloproteinase-1)
A prognostic marker in primary breast cancer. PMID: 15073104
May be useful as a prognostic marker in combination with uPA/PAI-1 and adds substantial positive information on the use of TIMP-1 as a prognostic marker in breast cancer. PMID: 15073104
   tPA-HCAb for Tissue-type plasminogen activator
A new prognostic marker in breast cancer. PMID: 3124957
   Topo11 alfa-HCAb for Topo LI-alpha
Overexpression appears to be linked with cellular dedifferentiation and potentially aggressive tumor phenotype in invasive breast cancer. PMID: 11174071

### TPt3-HCAb for TP53

TP53 mutation is a strong marker for the prediction of overall and disease-free survival in breast cancer, irrespective of nodal status. PMID: 10987229
   TPpa-HCAb for TPpA (Tissue polypeptide antigen)
A marker of central nervous system metastases of breast cancer. PMID: 2041052
   TPS-HCAb for TPS (Serum tissue polypeptide specific antigen)
A complementary tumor marker to CA 15-3 in the management of breast cancer. PMID: 11678313
Serum TPS at admission had a significant predictive value with regard to survival up to 12 months in breast cancer patients. PMID: 8687149

### TRACP-HCAb for TRACP

A useful marker of metastatic bone disease and response to treatment in breast cancer patients. PMID: 10650803
c-TRACP-5b-HCAb for TRACP 5b
Tartrate-resistant acid phosphatase 5b activity is a useful bone marker for monitoring bone metastases in breast cancer patients after treatment. PMID: 16537696, PMID: 15701839
Diagnostic sensitivity and specificity of TRAP 5b as a marker of skeletal metastases in patients with breast cancer were 82 and 87%, respectively. PMID: 15514730
TRACP 5b activity can be considered a surrogate indicator of bone metastasis in breast cancer patients. PMID: 15153786
uKPA-HCAb for UK-PA (Urokinase plasminogen activator)
Appears to be a new and independent prognostic marker in breast cancer. PMID: 2119883
uPA expression in breast cancer patients is under epigenetic control via methylation of its promoter. Determination of uPA promoter methylation can therefore serve as an early reliable indicator of uPA production in breast cancer patients. PMID: 15131040
   YB1-HCAb for YB-1 (Y-box binding protein 1)
A marker of tumor aggressiveness and response to adjuvant chemotherapy in breast cancer. PMID: 15703814

### YKL40-HCAb for YKL-40

Determination of serum YKL-40 can be used as a prognostic marker related to the extent of disease and survival of patients with recurrence of breast cancer. PMID: 7577068

### Other HCAbs for Breast Cancer Markers

FDGPET for FDG-PET for detecting recurrent breast cancer suspected from asymptomatically elevated tumor markers levels and has an important clinical impact on the management of these patients. PMID: 12324574
FBL for detecting FBL is associated with early manifestation of breast cancer and may be considered as a tool for the screening of breast cancer in high risk women. PMID: 9232610
Urinary testosterone is a prognostic indicator of early breast cancer recurrence in node-positive patients. PMID: 8400317

### Colorectal Cancer:

Colorectal cancer, also called colon cancer or bowel cancer, includes cancerous growths in the colon, rectum and appendix. It is the third most common form of cancer and the second leading cause of death among cancers in the Western world. Many colorectal cancers are thought to arise from adenomatous polyps in the colon. These mushroom-like growths are usually benign, but some may develop into cancer over time. The majority of the time, the diagnosis of localized colon cancer is through colonoscopy. Therapy is usually through surgery, which in many cases is followed by chemotherapy.

Camelid or shark can be immunized with the biomarkers associated with colon cancer. Heavy chain-only antibodies and their analogs that specifically bind to these biomarkers can be made using various methods such as, protease digestion, recombinant DNA technology and chemical means as described above. Heavy chain-only antibodies and their analogs that specifically bind to these biomarkers can be used in diagnosis, prognosis and/or treatment of colon cancer. Exemplary colon cancer biomarkers are shown below.

AFU (alpha-L-fucosidase)
Serum AFU activity appears to be a good prognostic factor of tumor recurrence in colorectal carcinoma. PMID: 12457030

### AM-3

A marker of dysplasia in the colonic adenoma-carcinoma sequence. PMID: 1350509

### Annexin II and Tenascin-C

Are overexpressed in advanced colorectal carcinoma and that they may be related to the progression and metastatic spread of colorectal carcinoma. PMID: 11745218

### ANAX3

overexpressed in colorectal tumoral tissues

### Arginase

Demonstrate higher clinical value in the early diagnosis of colorectal liver metastases than CEA and Ca 19-9. PMID: 15074017

### Bcl-2

A useful prognostic marker in Dukes' B colon cancer. PMID: 11456053

### Beta Catenin

Nuclear beta catenin expression is a potential prognostic factor in patients with colorectal cancer, and together with CK20, it could be used to identify colorectal carcinoma in the Hong Kong population. PMID: 14645698

### BMP4

over-expressed in colorectal tumoral tissues.

### CA-50

A clinically useful tool for monitoring of patients with colorectal cancer. PMID: 2451612

### CA 125

A clinically useful tumor marker in the management of colorectal carcinoma metastatic to the liver in patients with normal carcinoembryonic antigen. PMID: 10776987

### CA-195

Can be applied as complementary tumor marker in colorectal cancer. PMID: 2634457

### CA 242

Possible utility of CA 242 in monitoring the disease status, providing a rationale for future studies focusing on the longitudinal monitoring of colorectal cancer patients. PMID: 10365107

### Caspase 7

A new immunohistochemical marker of colonic neoplasia. PMID: 11381362

### CCAT25

Represents a highly promising marker for early detection of colorectal cancer in hereditary nonpolyposis colorectal cancer (HNPCC) germ line mutation carriers. PMID: 16166278
   CCSP-2 (Colon cancer secreted protein-2)
A novel candidate for development as a diagnostic serum marker of early stage colon cancer. PMID: 15580307

### CDC25B

A novel independent prognostic marker of colorectal carcinoma and that it may be clinically useful for selecting patients who could benefit from adjuvant therapy. PMID: 10850455

### CDX-2

A reliable marker of colorectal adenocarcinoma metastases to the lungs. PMID: 12548159
   CEA (carcinoembryonic antigen)
The measurement of CEA levels might be useful in monitoring chemotherapeutic response and in predicting the prognosis of patients with metastatic colorectal cancer. PMID: 11995458
Intraoperative determination of carcinoembryonic antigen levels in peritoneal washes could be a potentially predictive factor of a poor prognosis in patients with colorectal cancer. PMID: 12845971
CEA concentration in colonic effluent is a simple and practical biomarker for identification of patients at high risk for colorectal carcinoma (CRC). PMID: 10975292

### CMU10

A marker for colonic carcinoma and precancerous conditions. PMID: 7748075

### CR-1 (CCripto-1)

Plasma CR-1 might represent a novel biomarker for the detection of breast and colon carcinomas. PMID: 16951234

### Cyclooxygenase-2

Expression in the primary lesion may be a useful marker for evaluating prognosis and liver metastasis in patients with colorectal cancer. PMID: 11786771
   DD (plasma D-dimer)
Measurement of the preoperative DD level is thus considered to be useful for the preoperative staging of colorectal cancer. PMID: 9590700

### DDHN-MA

Urinary DHN-MA is a useful noninvasive biomarker for determining the risk of preneoplastic lesions associated with heme iron consumption and should be further investigated as a potential biomarker of colon cancer risk. PMID: 17119057
   DiAcSpm (di-acetyl spermine)
Urine DiAcSpm predicted the prognosis after colorectal cancer surgery more exactly than serum CEA. PMID: 15164601

### Dipeptidase 1

A candidate tumor-specific molecular marker in colorectal carcinoma./span> PMID: 15145522

### DPD (Dihydropyrimidine Dehydrogenase)

A useful marker for use with adjuvant chemotherapy with oral fluoropyrimidines after curative resection of colorectal cancer. PMID: 15309506
Tumor DPD level is an efficacious marker in oral 5-FU based-adjuvant chemotherapy for colorectal cancer; however, low tumor DPD predicts reduced survival in patients treated with curative surgery alone. PMID: 14744539

### E-Cadherin

Would be important predictive markers for lymph node metastasis in submucosal invasive colorectal carcinoma. PMID: 15785891

### E-selectin

The elevation of E-selectin alone or both E-selectin and sialyl Lewis A may be one of the useful indexes for more precise diagnosis of hematogenous metastases of human colorectal cancer. PMID: 11777210

### GGalectin-3

An independent factor for prognosis in colorectal cancer. PMID: 16080575
   GCC (Guanylyl cyclase C)
Reverse transcriptase PCR is a sensitive and specific technique for identifying tumor cells in extraintestinal sites and may be useful for staging and postoperative surveillance of patients with colorectal cancer. PMID: 16149873
   GLUT1 glucose transporter
Associated strongly with neoplastic progression in the colon, and assessment of the extent of GLUT1 immunostaining in colorectal carcinoma identifies patients with a poorer prognosis. PMID: 9655290

### GST-pi

A possible new marker for immunohistochemical detection of human colonic carcinoma and some adenomas. PMID: 3084412
   HECA (homologue of the Drosophila headcase protein)
May be an early-stage classifier of colorectal cancer that can discriminate between late- and early-stage disease. PMID: 16525680

### KKeratin 20

A specific marker of submicroscopic lymph node metastases in colorectal cancer: PMID: 10767714

### Ki67

Ki-67 labeling index in the mucosa adjacent to cancer might be a good marker for metastasis in colorectal cancer. PMID: 11876549

### L1

A new target gene for nt/beta-catenin-TCF signaling, is associated with tumor progression and poor survival in patients with colorectal cancer and may be clinically useful as a marker for poor prognosis. PPMID: 17211730

### Lactoferrin

A new marker for inflammatory gastrointestinal disorders and colon cancer. PMID: 8001286

### LCN2

Over-expressed in colorectal tumoral tissues.

### Metallothionein

Early marker in the carcinogenesis of ulcerative colitis-associated colorectal carcinoma. PMID: 12053227

### MMP-1

A prognostic marker for hematogenous metastasis of colorectal cancer. PMID: 10794801

### MMP-2/MMP-7/MMP-9/MMP-11

Activities of active MMP-2, MMP-7, MMP-11 and proMMP-9 in the bile may be useful markers for predicting liver metastasis in colorectal cancer. PMID: 11594775
   MnSOD (Manganese superoxide dismutase)
Immunohistochemical expression of MnSOD can give auxiliary clinical information for malignant potential of colorectal carcinoma. PMID: 12469142

### MUC1

Mature MUC1 mucins become ectopically expressed in colorectal carcinoma progressed to the metastatic stages and that mature MUC1 mucins may be a useful marker for advanced colorectal carcinoma. PMID: 7905449

### MYCL1

A useful prognostic factor of poor outcomes in colorectal cancer. PMID: 15014029
   NCC-ST 439o:p>
The determination of serum NCC-ST 439 in large bowel cancer might be useful in cancer staging and that NCC-ST 439, if used in combination with CEA, is particularly useful in diagnosing recurrences because of its improved diagnostic accuracy. PMID: 1914727
Serum NCC-ST-439 value was clinically useful for the diagnosis and monitoring for patients with colorectal cancers as a new distinct tumor marker. PMID: 2782914, PMID: 2549882
   NNMT (nicotinamide N-methyltransferase)
Serum levels may have significance in the early detection and in the management of patients with colorectal cancer. PMID: 16166432

### Osteopontin

Identified as colon cancer tumor progression marker. PMID: 14744111
A lead marker of colon cancer progression, using pooled sample expression profiling. PMID: 11929952
   p53
Detection of serum p53 antibody is expected to serve as a new genetic marker, determined by serological analyses, for aiding in the early diagnosis of superficial colorectal cancer and indicating its local curability after endoscopic treatment. PMID: 11706527
   PAI-1 (plasminogen activator inhibitor-1)
Might serve as a new parameter for the prediction of prognoses in colorectal cancer (CRC). PMID: 16091756

### PGP9.5

Expression is related to tumor progression and may be useful as a marker for invasive colorectal cancer. PMID: 11801558
   PKC (Protein kinase C)
A biological marker of risk of developing colorectal cancer or risk of bearing an asymptomatic tumor. PMID: 1860726
   pKi-67
Quantitatively determined pKi-67 mRNA can be a good and new prognostic indicator for primary resected colorectal carcinoma. PMID: 15449182

### PMEPA1

A transforming growth factor-beta-induced marker of terminal colonocyte differentiation whose expression is maintained in primary and metastatic colon cancer. PMID: 12670906
   pp53
Accumulation of p53 protein might have a favorable prognostic value in colorectal cancer, but it is not an independent prognostic factor. PMID: 17021749

### PRL-3

Expected to be a promising biomarker for identifying colorectal cancer patients at high risk for distant metastases. PMID: 15534108
   pS2
A possible diagnostic marker of colorectal carcinoma in ulcerative colitis. PMID: 10671663

### PSME3

A novel serum tumor marker for colorectal cancer (CRC) that may have significance in the detection and in the management of patients with this disease. PMID: 16893879

### SS100A4

Expression is associated with adverse clinical outcome. Inclusion of S100A4 expression status may enhance our accuracy to prognosticate in patients with colorectal cancer. PMID: 16615153

### SERCA2

May be a molecular determinant in the development and progression of colorectal cancer (CRC). PMID: 16861967

### SSKI

Amplification of SKI is a negative prognostic marker in early-stage colorectal cancer. PMID: 15153332

### SMAD4

A prognostic marker in colorectal cancer. PMID: 15814640
A predictive marker for 5-fluorouracil-based chemotherapy in patients with colorectal cancer. PMID: 12237773

### SMAD7

A prognostic marker in patients with colorectal cancer. PMID: 12584741

### STAT3

Expression of p-STAT3 is an important factor related to tumor invasion and poor prognosis of human colorectal adenocarcinoma. PMID: 16685378

### STRAP

A strong predictive marker of adjuvant chemotherapy benefit in colorectal cancer. PMID: 15720808

### Sucrase-isomaltase (SI)

An independent prognostic marker for colorectal carcinoma. PMID: 7497836

### TA90-IC

A new marker for advanced colon cancer. PMID: 10864342

### TAG-72

A novel tumor marker for colorectal cancer patients./span>PMID: 1892526
   TTCR gamma (T-cell receptor gamma)
A microsatellite marker for colorectal cancer. PMID: 11833498

### Telomerase

Useful both as a diagnostic as well as a predictive factor in colorectal cancer. PMID: 15190417

### TEM-8

A marker that identifies tumor associated micro-vessels in colon cancer. The levels of expression of TEM-8 in invasive colon cancer are linked to disease progression. This suggests that TEM-8 has significant prognostic and therapeutic values in colon cancer. PMID: 15498639

### Tetranectin (TN)

May be valuable as a prognostic variable in future studies evaluating new treatment strategies for colorectal cancer. PMID: 12529016

### TGF-beta1

Active TGF-beta1 might be used as a tumor marker for colorectal cancer. PMID: 11802214

### Thymidine Kinase 1 (TK1)

TK1-LI showed more potential as a proliferating marker in colorectal carcinoma than PCNA-LI, especially for evaluating high-risk tumor grade and advanced stage in colorectal carcinoma. PMID: 11205225
   VEGF (vascular endothelial growth factor)
A useful complementary tumor marker in patients with colorectal cancer. PMID: 16741643, PMID: 16645748
A predictive marker of rectal tumor response to preoperative radiotherapy. PMID: 16222693

### VEGF-D

VEGF-D expression, but not that of its receptor VEGFR-3, is an independent prognostic indicator in colorectal carcinomas (CRC). PMID: 11912138
   Vitamin D receptor
The level of vitamin D receptor correlates with the degree of differentiation in human colon cancer cell lines and may serve as a useful biological marker in predicting clinical outcome in patients. PMID: 8393379

### Other Colorectal Cancer Markers

Detection of circulating tumor cells erioperatively by immunobead-PCR provides a sensitive prognostic marker for recurrent and metastatic colorectal cancer. PMID: 8612201
Methylation of serum DNA is an independent prognostic marker in colorectal cancer. PMID: 17189406
A combination of serum hepatocyte growth factor and carcinoembryonic antigen tests might be useful for selecting patients with aggressive diseases in Dukes A and B colorectal cancer classification. PMID: 16990975
This innovative membrane array technique with a multiple mRNA marker panel can significantly improve the diagnosis rate of early colorectal cancer. PMID: 16391796
A mini-array of multiple TAAs which includes Calnuc might provide a novel non-invasive approach to enhance antibody detection for colon cancer diagnosis. PMID: 17390015
An increment of soluble FAS/soluble FASL ratio after treatment could be an excellent marker of chemosensitivity in colorectal cancer. PMID: 16000573
Fecal sphingomyelinase activity could really reflect the human intestinal mucosa enzyme level and could represent a new marker for human colorectal adenocarcinoma, mainly taking into account its early appearance in intestinal neoplasms. PMID: 15824156
6 months postoperatively serum CEA is a better prognostic marker than corresponding serum and plasma VEGF. However, high serum VEGF within high serum CEA was an even better predictor of overall survival than high serum CEA alone. PMID: 14968945
An assay of fecal DNA integrity may be a useful biomarker for the detection of colorectal cancer (CRC). PMID: 12816901
Lipid peroxidation as additional marker in patients with colorectal cancer. Results of a preliminary study. PMID: 12364818
Hyaluronic acid (HA) may provide additional information to that given by other biochemical markers currently used in colorectal cancer. PMID: 11122186
The evaluation of circulating testosterone could be a new and more sensitive assay for diagnosis and follow-up of colorectal carcinoma in males, especially in patients with normal levels of carcinoembryonic antigen. PMID: 8295527
Tumor Budding is a reliable biological prognostic variable to identify higher malignancy potential. Scoring system using tumor budding and N stage showed better prognostic stratification in stage-III rectal carcinoma. PMID: 17216219
Budding is a pathological marker suggesting high malignant potential and decreased postoperative survival in patients with colorectal mucinous carcinoma. PMID: 12451038
Mandibular osteomas are probably genetic markers of the development of sporadic colorectal carcinoma. PMID: 8381556

### Ovarian Cancer:

Ovarian cancer is a malignant ovarian neoplasm (an abnormal growth located on the ovaries). Often, this cancer is detected at an advanced stage when it is too late to treat. Early detection is a must for early intervention of a disease. Camelid and shark heavy chain only antibodies and their analogs may be used to detect ovarian cancer biomarkers.

Camelid or shark can be immunized with the biomarkers associated with ovarian cancer. Heavy chain-only antibodies and their analogs that specifically bind to these biomarkers can be made using various methods such as, protease digestion, recombinant DNA technology and chemical means as described above. Heavy chain-only antibodies and their analogs that specifically bind to these biomarkers can be used in diagnosis, prognosis and/or treatment of ovarian cancer. Exemplary ovarian cancer biomarkers are shown below.

### Alpha 1 AT

A useful tumor marker in diagnosing ovarian cancer (OC). PMID: 2472494

### Alpha(v)-integrin

A novel prognostic marker in advanced-stage ovarian carcinoma. PMID: 11751504
   Alpha (v) beta(6) Integrin
Associated with epithelial ovarian cancer and that a gradual increase in the expression of the molecule may be a correlative index of the progression of this disease. PMID: 12364570

### ATP7B

May be considered as a novel chemoresistance marker and that inhibitor(s) of ATP7B might be useful, in patients with ovarian carcinoma treated with cisplatin-based chemotherapy. PMID: 12216079

### B2M (Beta-2-Microglobulin)

Combination of b2m and CA-125 tumor markers seems to be a suitable tool for follow-up ovarian cancer patients under and after treatment. PMID: 1585745

### Beta III Tubulin

Assessment of beta III tubulin could be useful to identify poor prognosis ovarian cancer patients to more aggressive and/or targeted therapy. PMID: 16675570

### CA54/61

Is of clinical value as a new tumor marker for ovarian cancers, including mucinous tumors. PMID: 1737381

### CA 72-4

Measurement of CA 72-4 be combined with measurement of CA 125, so as to provide a better sensitivity and specificity in monitoring an ovarian cancer. PMID: 2232183
   CA125 (tumor marker Cancer Antigen 125)
Currently widely applied in the management of patients with ovarian cancer. PMID: 12088336
Though not ovarian cancer specific, is widely used for the evaluation of suspected and under-treated ovarian cancer. PMID: 15668638, PMID: 16126266
The combination of FDG-PET and CA125 titer is useful for the accurate detection of recurrence for epithelial ovarian cancer. PMID: 16515575
A mucin commonly employed as a diagnostic marker for epithelial ovarian cancer. PMID: 12734200
A rise in CA 125 during or after treatment, however, is almost always associated with progression of the disease (ovarian cancer). PMID: 9477746
A glycoprotein and a commonly used tumor marker in ovarian carcinoma. PMID: 16724349
Useful to follow-up of patients whose ovarian carcinoma had already been diagnosed and suffered surgery procedures followed for chemotherapy. PMID: 8731596
The most sensitive marker for epithelial ovarian cancer, but the concomitant measurement of TATI could be of benefit in both differential diagnosis of adnexal masses and monitoring of response of epithelial ovarian cancer to treatment. PMID: 1780685
Determination of serum CA 125 should be mandatory in the follow-up investigation of women with epithelial ovarian carcinomas. PMID: 3480655
Chemotherapy alone is capable of lowering CA-125 serum levels. This tumor marker may be of great advantage in diagnosis and follow-up of ovarian malignancy. PMID: 3479814
Practical application of CA 125 proved to be useful for the early detection of ovarian cancer and confirmation of the complete disappearance of any tumor. PMID: 3461071
Routine determination of CA 125 appears advisable in the control of patients with ovarian carcinoma on account of the high sensitivity and specificity during follow-up. PMID: 2408962
The measurement of CA 125 in sera was considered to be significant in the diagnosis of ovarian cancer. PMID: 6595320
An excellent marker in the management of patients with epithelial ovarian cancers. PMID: 2737852

### CA125 II

Has higher precision than that of CA125 when it is used for the screening test. In conclusion, CA125 II is a better tumor marker than conventional CA125. PMID: 7737582

### CA602

May be a useful serum tumor marker for ovarian cancer as a substitute for CA125. PMID: 2170549
   caGT (cancer-associated galactosyltransferase antigen)
Will be a useful tumor marker for ovarian cancers, especially for clear cell carcinoma. PMID: 2105162

### CASA or YKL-40

Low serum levels of tetranectin, or high serum levels of CASA or YKL-40, are associated with increased risk of second-line chemoresistance in patients with ovarian cancer. PMID: 17013795

### Cathepsin B

Serum cathepsin B-like activity may be helpful in the preoperative differential diagnosis between ovarian carcinomas and benign ovarian or uterine tumors. PMID: 9166974

### CD24

Expressed in ovarian cancer and is a new independent prognostic marker of patient survival. PMID: 12368195

### CD34

A useful marker in determining tumor neovascularisation which might be of prognostic relevance in patients with ovarian cancer. PMID: 10470188
   c-Ets1
A promising marker in epithelial ovarian cancer. PMID: 11836635
   CKB (creatine kinase B)
Up-regulated in ovarian cancer cells in vitro and in vivo and that CKB enzyme activity is significantly elevated in sera from ovarian cancer patients, including those with stage I disease. These findings suggest a potential role for CKB as a marker for early diagnosis. PMID: 15589584

### COX-1

A suitable marker for the monitoring and diagnosis of ovarian cancer patients, when used alone or in combination with CA 125. PMID: 7589737
   EMMPRIN (extracellular matrix metalloproteinase inducer)
A novel marker of poor outcome in serous ovarian carcinoma. PMID: 12705637
   Ep-CAM (epithelial cell adhesion molecule)
An independent prognostic marker for reduced survival of patients with epithelial ovarian cancer. PMID: 16678891

### Ets-1

A novel marker of poor survival in ovarian carcinoma. PMID: 11297247
   GAT (galactosyltransferase associated with tumor)
A newly developed tumor marker of ovarian carcinoma. PMID: 8129392, PMID: 8434966
The lower positive rate of GAT in endometriosis, when compared with the positive rate of other markers, suggests the usefulness of GAT in distinguishing malignant ovarian tumors from benign ovarian tumors. The use of GAT in a combination assay is expected to overcome the disadvantages of CA602 or CA125. PMID: 8434967
   GEP (Granulin-epithelin precursor)
A novel prognostic marker in epithelial ovarian carcinoma. PMID: 15139056
   GT-II (Galactosyltransferase isozyme II)
A new tumor marker for ovarian cancers—especially for clear cell carcinoma. PMID: 2511259

### HER-2

Appeared to influence the outcome of advanced ovarian cancer patients included in a clinical trial with prolonged follow-up, thereby suggesting that HER-2 is a potential target for treatment of this cancer. PMID: 14679128
   hK8 (Human kallikrein 8)
An independent marker of favorable prognosis in ovarian cancer. PMID: 16533772
   hK10 (Human kallikrein)
May be a potential new serological marker for ovarian cancer diagnosis and monitoring. PMID: 11282101
   hK13 (Human kallikrein 13)
An independent marker of favorable prognosis in ovarian cancer. PMID: 14966091

### HLA-G

Aprognostic indicator in advanced-stage ovarian cancer in effusions. PMID: 15589578
   HNF-1beta
Is likely to be helpful for the diagnosis of CCC in the peritoneal fluid. PMID: 17351940
   IAP (immunosuppressive acidic protein)
May also be a useful follow-up marker for patients with ovarian cancer (particularly, for the early detection of recurrence). PMID: 3701144
Not only an excellent clear cell carcinoma (CCC)-specific molecular marker but also a molecular target for therapy of ovarian clear cell carcinoma (OCCC). PMID: 14633622

### IGFBP-2

May therefore be an important additional prognostic marker in ovarian cancer. PMID: 15014034
   KLK9 (kallikrein gene 9)
A potential new independent favorable prognostic marker for early stage, low-grade, optimally debulked ovarian cancer patients. PMID: 11691797
   M-CAM (melanoma cell adhesion molecule)
A marker of poor prognosis in epithelial ovarian cancer (EOC). PMID: 16804906
   M-CSF (Macrophage colony-stimulating factor)
A marker for ovarian cancer. Determination of serum levels can be useful in detecting ovarian cancer, particularly in combination with CA 125. PMID: 8233270

### Mesothelin

A new tumor marker for the differential diagnosis of epithelial ovarian carcinoma and a prognostic factor for the outcome of epithelial ovarian carcinoma patients. PMID: 17214332
   MMP-2 (matrix metalloproteinase-2)
A potential marker of prognosis for epithelial ovarian cancer. PMID: 11748988
   nm23-H1
May serve as a potentially valuable marker for ovarian tumors. PMID: 7763042
   p53
Could be used as a marker for predicting the response to chemotherapy of ovarian cancer. PMID: 9797696
   P-III-P (type III procollagen peptide)
Very useful as tumor marker in diagnosis of malignant ovarian tumors, while it was not aided to detect the early cancers. PMID: 2778385

### P-glycoprotein

A predictor of response and survival in a uniformly treated and followed cohort of advanced ovarian cancer patients. PMID: 10810398
   PP-4 (Placental protein 4)
A possible tumor marker in ovarian tumors. PMID: 1836196

### Progesterone

May be used as a tumor marker in “nonendocrine” ovarian tumors. PMID: 6884825

### Progesterone Receptor (PR)

An independent marker, with its overexpression associated with a favorable prognosis in women with ovarian cancer. PMID: 15721410

### Prostasin

Overexpressed in epithelial ovarian cancer and should be investigated further as a screening or tumor marker, alone and in combination with CA 125. PMID: 11584061

### PUMP-1

Frequently overexpressed in ovarian tumors and may contribute to its invasive nature or growth capacity. PMID: 10050107
   sialyl SSEA-1 antigen
Appears to be a useful tumor marker for the diagnosis of ovarian cancer, especially when measured simultaneously with CA 125, CA19-9, TPA, ferritin and IAP. PMID: 2566639

### SM047

Strongly expressed in most ovarian serous adenocarcinomas and in other female genital tract adenocarcinomas, with the exception of ovarian mucinous tumors. The antibody may be useful in confirming the ovarian origin of an adenocarcinoma when used as part of a larger panel. PMID: 11422498
   STN antigen (serum sialyl Tn antigen)
A positive STN antigen level in sera is an independent predictor of poor prognosis in ovarian cancer. PMID: 1541859
Positive STN antigen level in sera is an independent predictor of poor prognosis in epithelial ovarian cancer. PMID: 1678592

### TAG-72

Might be considered a (progression) marker between the subgroup of benign and malignant serous ovarian tumors. PMID: 9167042

### Thymidine Phosphorylase (TP)

Might be useful in diagnostic characterization of ovarian cancer. PMID: 15262124

### TNF Receptors

In peritoneal fluid, measurement of soluble TNF receptors, and particularly of p75, has an increased sensitivity and accuracy over CA125 in distinguishing ovarian cancer from benign pelvic masses. PMID: 7729731

### Topoisomerase II

a prognostic marker for survival in ovarian cancer. PMID: 11426969
   tPA (tissue plasminogen activator)
High concentration of plasma tPA was an independent marker for poor prognosis in patients with ovarian cancer in our study. Plasma tPA did, however, not discriminate between benign and malignant adnexal lesions. PMID: 14529669

### VSGP/F-spondin

A potential diagnostic marker or target for developing therapeutic strategies to treat ovarian carcinoma. PMID: 16103746

### WT-1

A highly sensitive and specific marker of serous carcinomas of ovarian surface epithelial origin. PMID: 15354736
In assessment of effusion specimens with metastatic carcinoma, nuclear reactivity for WT1 is highly suggestive of an ovary primary tumor. PPMID: 11954027
Expression of WT1 gene may be indicative of an unfavorable prognosis in patients with advanced serous epithelial ovarian carcinoma. PMID: 16606472
   YB-1 (Y box-binding protein-1) and P-gp (P-glycoprotein)
Co-expression of YB-1 and P-gp emerged as a promising relevant biomarker for unfavorable prognosis in ovarian cancer. PMID: 15099935

### YKL-40

A prognostic tumor marker in recurrent ovarian cancer. PMID: 12694127

### Other Ovarian Cancer Markers

FDG-PET is a useful technique to detect recurrent ovarian cancers for patients suspected of recurrent ovarian cancers due to asymptomatically elevated serum levels of CA-125 antigen. PMID: 12458332
Indoleamine 2,3-dioxygenase screened with the GeneChip was positively associated with paclitaxel resistance and with impaired survival in patients with serous-type ovarian cancer. PMID: 16115948

### Cervical Cancer:

Cervical cancer is a malignancy of the cervix. It may present with vaginal bleeding but symptoms may be absent until the cancer is in its advanced stages, which has made cervical cancer the focus of intense screening efforts utilizing the Pap smear. Most scientific studies have found that human papilloma virus (HPV) infection is responsible for virtually all cases of cervical cancer. Treatment consists of surgery (including local excision) in early stages and chemotherapy and radiotherapy in advanced stages of the disease.

Camelid or shark can be immunized with the biomarkers associated with cervical cancer. Heavy chain-only antibodies and their analogs that specifically bind to these biomarkers can be made using various methods such as, protease digestion, recombinant DNA technology and chemical means as described above. Heavy chain-only antibodies and their analogs that specifically bind to these biomarkers can be used in diagnosis, prognosis and/or treatment of cervical cancer. Exemplary cervical cancer biomarkers are shown below.

Beta-CF (Beta-core fragment)
A fragment of the hCG beta-subunit missing its carboxyterminal peptide. The determination of urinary beta-CF may provide a useful tool in monitoring the response to treatment in patients with cervical cancer. PMID: 1545171

### CD34

A marker for evaluating angiogenesis in cervical cancer. PMID: 15991838

### CDH1/CDH13

Detection of aberrant methylation of CDH1/CDH13 may be of potential use as a marker for selecting cervical cancer patients at high risk for relapse who could benefit from additional systemic therapy. PMID: 14750164

### ERCC1

A molecular marker of cisplatin resistance in human cervical tumor cells. PMID: 11008208
   HIF-1alpha
a strong independent prognostic marker in early stage cervical cancer. PMID: 10987269

### HPV-16 L1

Antibodies to HPV-16 L1 were found to be an independent prognostic factor for overall survival in patients with cervical cancer. PMID: 11967495

### HPV DNA

Serum HPV DNA might be a useful additional marker for early detection of recurrence in cervical cancer patients. PMID: 14643453
In patients with cervical cancer, an approach based on a PCR test for HPV DNA in tumor-free regional lymph nodes may allow early identification of women at high risk for relapse who should receive adjuvant treatment. PMID: 9166495

### Id-1

An independent prognostic marker in early-stage cervical cancer. PMID: 11479201
   p16
A useful marker for the detection of the adenocarcinoma of the cervix uteri and its precursors. PMID: 12548164
   p21WAF1/CIP1
Expression of p21 WAF1/CIP1 correlated with a favorable prognosis for patients with cervical adenocarcinoma and may serve as a useful marker of survival in cases of this disease. PMID: 9635534
   PP-4 (placental protein 4)
A recently characterized glycoprotein from human placenta, can be regarded as a tumor associated protein which most likely can serve as tumor marker in cervical and endometrial cancer. PMID: 1833844
   SCC antigen (squamous cell carcinoma antigen)
A new tumor marker for cervical carcinoma. PMID: 2591447
Might be helpful in the control of the primary therapy and follow-up of cervical cancer patients. PMID: 2777050
Proved to be a valuable tumor marker for the follow up of cervical, vaginal, and vulvar cancer. PMID: 2721888
   Tn antigen (Tn-Ag)
A combination of estimations of the degree of cancer involvement in the cervical stroma and Tn-Ag expression seems the most useful for predicting the prognosis of patients with cervical cancer. PMID: 8508399

### TPS

Especially in the combination with SCC may be useful in the diagnosis and estimation of stage of disease of patients with cervical carcinoma. PMID: 11320545

### Tu M2-PK

Can be used as a tumor marker in follow-up of patients with cervical carcinoma. PMID: 15210041
   UGF (urinary gonadotropin fragment) and SCC (squamous cell carcinoma antigen)
Both UGF and SCC be used to monitor therapy and to detect recurrences of cervical and vulvar cancers. PMID: 2354828

### Bladder Cancer:

Bladder Cancer refers to any of several types of malignant growths of the urinary bladder. It is a disease in which abnormal cells multiply without control in the bladder. The most common type of bladder cancer begins in cells lining the inside of the bladder and is called urothelial cell or transitional cell carcinoma (UCC or TCC). Approximately 20% of bladder cancers occur in patients without predisposing risk factors. Bladder cancer is not currently believed to be heritable (i.e., does not “run in families” as a consequence of a specific genetic abnormality).

Camelid or shark can be immunized with the biomarkers associated with bladder cancer. Heavy chain-only antibodies and their analogs that specifically bind to these biomarkers can be made using various methods such as, protease digestion, recombinant DNA technology and chemical means as described above. Heavy chain-only antibodies and their analogs that specifically bind to these biomarkers can be used in diagnosis, prognosis and/or treatment of bladder cancer. Exemplary bladder cancer biomarkers are shown below.

Beta-hCG (beta human chorionic gonadotrophin)
For T2-T4 bladder tumors, an elevated pre-treatment level of urinary beta-hCG is a marker of poor prognosis and may prove useful in deciding appropriate therapy. PMID: 8653319
Measurement of serum and/or urine beta HCG appears to be an efficient diagnostic marker for the presence of distant metastases in bladder carcinoma. PMID: 2478247
   Beta-2-microglobulin
A differentiation marker in bladder cancer. PMID: 3534313

### BLCA-1

A urine based marker of bladder cancer which may be useful for the detection of this disease. PMID: 15947579

### BLCA-4

A bladder cancer specific biomarker, is present in urine samples from patients with bladder cancer, but not in samples from healthy individuals. PMID: 16360453
A bladder cancer marker that is highly specific and occurs early in the development of the disease. PMID: 14977841
Urinary BLCA-4 determination appears to have high potential as a test for screening and monitoring bladder cancer in the general population and in groups at high risk for the disease, such as those with spinal cord injury. PMID: 10953114
   BTA (Bladder tumor antigen)
In combination with urine cytology is a more useful way for diagnosing TCC of the bladder. PMID: 1891992
   CA19-9 (Carbohydrate antigen 19-9)
Promising for use as a biomarker for the detection and monitoring of low-grade and low-stage bladder cancer, with the proviso that patients to be tested should be free of infection. PMID: 15764245
   c-erbB-2
The use of c-erbB-2 gene amplification, together with tumor grade and stage, could provide an accurate basis for determining the prognosis of bladder cancer. PMID: 10792078

### Clusterin

Serum and urine clusterin can differ between bladder cancer patients and the control group. Urine clusterin could be the possible laboratory marker of bladder cancer. PMID: 16830064

### CYFRA 21-1

Patients with transitional cell cancer of the bladder with evidence of distant metastases showed a significant increase in serum CYFRA 21-1. During chemotherapy CYFRA 21-1 appears to be a potentially sensitive and useful indicator for monitoring treatment response. PMID: 16217281

### Cytokeratin-18 (CK18)

It is clear that serum cytokeratin-18 level increases in patients with bladder cancer. However, it can only be useful as a tumor marker in the diagnosis of T(3) and higher staged tumors. This study indicated that cytokeratin-18 does not have any diagnostic value in lower stage bladder cancers. PMID: 12135697

### Cytokeratin-20 (CK20)

A potential marker for bladder cancer, and is significantly more sensitive than urinary cytology. PMID: 9817302

### DD23

Quantification in single cells may be particularly useful in targeting cystoscopic intervention for recurrence monitoring and, because of its high specificity, could be a tool for bladder cancer screening in high-risk groups. PMID: 8959319
Expression can be used as an adjunct to cytopathologic evaluation to enhance the sensitivity of urinary cytology detection of TCC. PMID: 12639642
   EMA (Epithelial membrane antigen)
Could be a valuable indicator for histological grading or staging in pathological diagnosis and for predicting the survival of bladder cancer patients. PMID: 3303592

### ERCC1

A novel prognostic marker in advanced bladder cancer patients receiving cisplatin-based chemotherapy. PMID: 17229776

### F-actin

Could be an early and sensitive marker for bladder cancer detection and risk prognostication. PMID: 2032215

### FAS

a promising novel urinary marker for the detection of recurrent superficial bladder cancer. PMID: 16541433
   FBP (fucose-binding proteins)
Degree of expression of FBP binding sites correlates with the increased metastatic potential of bladder cancer and with poor patient survival times. PMID: 8339222

### GNAS1

A novel independent prognostic marker for clinical outcome supporting a functional role of G(alpha)s in bladder cancer progression. PMID: 15824158

### H19

Can be used as a tumor marker in human bladder carcinoma, where its expression indicates a more malignant potential. PMID: 7855987
Hyaluronic acid (HA), a glycosaminoglycan and known to promote tumor cell adhesion and migration, and its small fragments stimulate angiogenesis, is a new sensitive and specific urine marker for bladder cancer. PMID: 9044859

### Hyaluronan (HA)

HA in addition to being one of the best markers for the initial evaluation of bladder carcinoma can be used to determine the presence of a residual tumor. This is associated with poor prognosis. PMID: 16310928

### Hyaluronidase

A diagnostic urine marker for high-grade bladder cancer. PMID: 9044860
   hTERT
Expression in urine sediments represents a reliable tool for the detection of primary urothelial neoplasms, equally specific, yet far more sensitive, than conventional cytology. PMID: 12893365
   Lewis X antigen
Lewis X antigen on exfoliated bladder cells enhances the detection of urothelial tumor cells, particularly from low grade and low stage neoplasms. PMID: 2405185
   MMP-2 (matrix metalloproteinase-2)
MMP-2 protein overexpression may be an independent prognostic biomarker for bladder cancer progression. PMID: 14624933

### Neutral Endopeptidase

Will serve as a new tumor marker for bladder cancer as well as acute lymphatic leukemia. PMID: 7627835

### NMP22 (Nuclear Matrix Protein 22)

Recurrence marker in bladder cancer. PMID: 10900760
Urinary NMP22 is a useful tool for the screening of urothelial cancer in patients with microscopic hematuria. PMID: 9170522, PMID: 9170521
Urinary NMP22 is a useful diagnostic marker as a substitute for voided-urine cytology for the surveillance of urothelial cancer. PMID: 9076459

### OCT-4

An embryonic stem cell marker, is highly expressed in bladder cancer. PMID: 17205510
   p53
Can be used as a tumor marker for bladder cancer. PMID: 15503000
p53 nuclear immunostaining yields clinically relevant information and may be a useful tool for selecting patients with superficial bladder cancer who might be resistant to BCG. PMID: 15041112

### Prothymosin-alpha

Urine prothymosin-alpha has the potential of being a useful tumor marker for the detection and follow-up of bladder cancer. PMID: 16461079

### Survivin

A sensitive marker for the noninvasive diagnosis of bladder cancer. PMID: 14713774
   T antigen
Useful to predict the response of bladder tumors to treatment with bacillus Calmette-Guerin and interleukin-2. PMID: 2477561

### Telomerase

An important marker in the diagnosis of bladder cancer. PMID: 15153335
The presence of telomerase in bladder washes may be a specific marker of bladder cancer, especially in low-grade tumors. PMID: 9533519
Can be determined in voided urine samples of patients with superficial bladder cancer. It has a higher sensitivity and specificity than conventional urinary cytology and is a good marker for diagnosis and follow-up of these patients. PMID: 10851728

### Thymidine Kinase 1 (TK-1)

A proliferation marker for determining prognosis and monitoring the surgical outcome of primary bladder carcinoma patients. PMID: 16391869

### Thymidine Phosphorylase (TP)

A prognostic marker for predicting recurrence in primary superficial bladder cancer. PMID: 16820903
   TPA (Tissue polypeptide antigen)
A useful marker not for the early detection of bladder cancer but for the monitoring of the efficacy of a treatment. PMID: 8694544

### Transferrin Receptors (TFR)

TFR activity in low grade superficial bladder tumors is a useful marker for predicting the recurrence rate. PMID: 2004229
   UCA1 (urothelial carcinoma associated 1)
Avery sensitive and specific unique marker for bladder cancer. PMID: 16914571

### Uroplakin II (UP II)

Might be a more useful marker than CK 20 for detecting micrometastases of bladder cancer in the pelvic lymph nodes, although a greater number of patients and longer follow-up are needed to come to a definitive conclusion. PMID: 16280744

### XIAP

A prognostic marker of early recurrence of nonmuscular invasive bladder cancer. PMID: 17439739

### Other Bladder Cancer Markers

Resting NOR had a predictive value in the prognosis of patients with invasive bladder tumor. PMID: 11564895
Microvessel density (MVD) in bladder carcinoma correlates with grade, stage and malignant potential of the tumor. PMID: 15783115
Hydronephrosis is a prognostic marker in bladder cancer in a cystectomy-only series. PMID: 16904815
The Ce6-PVP formulation appeared to have the potential as a fluorescent marker for fluorescence diagnosis of human bladder cancer. PMID: 16516376

### Circulating Tumor Cells (CTCs):

Camelid or shark can be immunized with the biomarkers associated with circulating tumor cell. Heavy chain-only antibodies and their analogs that specifically bind to these biomarkers can be made using various methods such as, protease digestion, recombinant DNA technology and chemical means as described above. Heavy chain-only antibodies and their analogs that specifically bind to these biomarkers can be used in diagnosis, prognosis and/or treatment of cancer. Exemplary bladder cancer biomarkers are shown below.

| Antigens | Application |
|---|---|
| MUC-1 | Metastatic Breast Cancer |
| VCAM-1 | Breast Cancer |
| EpCAM | Breast, Colon, Stomach, Pancreatic, lung and |
| | Gastric Carcinoma |
| CD44 | Gastric and colon cancers |
| E-Cadherin | Gastric cancer. Therapeutic applications for breast |
| | cancer |
| VEGF | Colon and breast cancers |
| bFGF | Breast, head, and neck cancers |
| sFasL | Bladder and Gastric Carcinomas |
| sFas (CD95) | Leukemia, colon, breast and bladder cancers |
| p53 HCAb | Breast, lung, colon, gastric, oral, and |
| | lymphoreticular cancers |
| Bcl-2 Protein | Thyroid, prostate, colon, uterine cervix, bladder, |
| | and breast cancers |
| Cyclin D1 | Ductal breast cancer, head and neck carcinoma |
| Cyclin E | Breast cancer |
| TGF-beta1 | Bladder cancer, ovarian carcinoma, live cancer, and |
| | prostate cancer |
| TNF-alfa | Pancreatic cancer |
| C-erbB2 (HER-2) | Breast, ovarian, gastric, endometrial carcinoma, |
| | adenocarcinoma, and prostate cancer |
| EGFR | Breast, brain, bladder, and head cancer. Therapeutic |
| | target for c-HCAb |
| IGF-1 and IGF-1R | Breast, prostate, colon and lung cancers. IGF-1R is |
| | a therapeutic target |
| IL-2R | Breast cancer, leukemia, head and neck cancer |
| Ras Protein | Colon, bladder, gastric, and pancreatic cancers. |
| | Ovarian, endometrial, and duodenal |
| | adenocarcinomas |
| C-Myc | Colon carcinoma and adenomatous |

### Brain Tumors/Lesions/Plaques:

A brain tumor is any intracranial tumor created by abnormal and uncontrolled cell division, normally either found in the brain itself (neurons, glial cells (astrocytes, oligo-dendrocytes, ependymal cells), lymphatic tissue, blood vessels), in the cranial nerves (myelin-producing Schwann cells), in the brain envelopes (meninges), skull, pituitary and pineal gland, or spread from cancers primarily located in other organs (metastatic tumors). Primary (true) brain tumors are commonly located in the posterior cranial fossa in children and in the anterior two-thirds of the cerebral hemispheres in adults, although they can affect any part of the brain. In the United States in the year 2000, it was estimated that there were 16,500 new cases of brain tumors which accounted for 1.4 percent of all cancers, 2.4 percent of all cancer deaths, and 20-25 percent of pediatric cancers. Ultimately, it is estimated that there are 13,000 deaths/year in the USA due to brain tumors.

Alzheimer's disease (AD) is sixth leading cause of death in the US. There are estimated 5.3 million Alzheimer's patients, with one new case being diagnosed every 70 seconds. AD is one of the most devastating diseases which deteriorate brain slowly but progressively to the point that the patient becomes non-functional. Neither there is a diagnostic test for AD nor any treatment.

Other brain diseases include: Parkinson's disease, Lyme's disease and Cysticercosis.

### Detecting Fetal Down Syndrome Biomarkers

In one embodiment, shark and camelid heavy chain only antibodies and their analogs can be used in capturing circulating fetal cells in a pregnant women's blood and diagnosing genetic disorders of the fetus such. Exemplary genetic disorder includes Down syndrome. Arginine/serine rich splicing factor 4 (RSSF4) is one of the biomarkers found only in the amniotic fluid of Down syndrome fetuses and not in normal pregnancies (Michael P E et al., Electrophoresis, 27, 1169 (2006)], transthyretin (TTHY), alpha-1-macroglobulin (AMP), ataman (FAN) and Apo lipoprotein (APE) [Prenatal Diag., 28, 691 (2008)]. Other serum proteins which are upregulated in Down pregnancies are serum amyloid P-component (SAMP) and alfa-1-antitrypsin (ANITA). Accordingly, detecting one or more of the biomarkers such as RSSF4, TTHY, AMP, FAN, APE, SAMP, ANITA will provide a unique marker for diagnosing Down syndrome the same day the pregnant mother visits her doctor without having to wait for karyotyping results which can take up to three weeks. The detection of these biomarkers will facilitate the development of a non-invasive, safe test for Down syndrome.

### Detection and Treatment of Brain Diseases;

Camelid or shark can be immunized with the biomarkers associated with BrainTumors/Lesions/Plaques. Heavy chain-only antibodies and their analogs that specifically bind to these biomarkers can be made using various methods such as, protease digestion, recombinant DNA technology and chemical means as described above. Heavy chain-only antibodies and their analogs that specifically bind to these biomarkers can be used in diagnosis, prognosis and/or treatment of BrainTumors/Lesions/Plaques. Exemplary BrainTumors/Lesions/Plaques biomarkers are shown below.

Exemplary biomarkers for brain tumor include:

Endosialin (tumor endothelial marker 1, TEM1)
Abundantly expressed in highly malignant and invasive brain tumors. PMID: 15624764
   PGD-S (Prostaglandin D synthetase)
A 30 kDa glycoprotein also known as beta-trace protein that catalyzes the formation of prostaglandin D2 (PGD2) from PGH2, is a potentially useful marker for brain tumor. PMID: 9844724
   PV-1 (Plasmalemmal vesicle associated protein-1) represents a novel marker of brain tumor angiogenesis and integrity of the blood-brain barrier and is a potential therapeutic target. PMID: 16278383

Exemplary biomarkers for Alzheimer's disease include:

ALZAS, Aβ₄₂, Tau, DJ-1, Apo-E, GSK-3, Cystatain, Apo-A1, VGF Protein, beta and gamma-secretases and Bax-1
Cytokeratin 1 through 20:

In one embodiment, the invention provides a method for the development of shark and camelid heavy chain only antibodies and their analogs for use as immunohistochemical agents for cell surface, cytoplasmic and/or nuclear proteins for the identification of pathological cells from bodily fluids (blood, urine, saliva, semen, mucus, tears, etc) and tissues, but not limited to, cancer cells, bacterial cells including anthrax, viral cells, including SARS, HIV, HBV, and HPV, neurological cells, cardiac cells, fetal cells and cells of the autoimmune diseases. It is known that all epithelial neoplasms express cytoskeleton proteins of the cytokeratin (CK) family. Shark and camelid heavy chain only antibodies and their analogs for CK1 through CK20 will be developed to distinguish between normal and neoplastic cells with much higher sensitivity and specificity than the conventional monoclonal antibodies.

High serum levels of CD44 have been detected in some solid tumors such as advanced gastric, colon, ovarian cancers, and non-Hodgkin lymphoma and B-cell chronic lymphocytic leukemia. CD44 is a adhesion molecule present on leukocytes. In other embodiment of the invention, single-domain antibodies for cell adhesion molecules including, but not limited to, CD44, VCAM-1, and ICAM-1 (aka: EpCAM) will be developed to improve sensitivity and specificity of detection of various forms of cancer.

### Camel and Shark Mini-, Micro-, and Nano-Antibodies Against Cell-Adhesion Molecules for Isolating and Identifying Rare Cells (Cancer and Fetal Cells).

In other embodiments, the invention provides camelid and shark heavy chain only antibodies and their analogs for all the cell surface molecules known as cell adhesion molecules or the cluster of differentiation proteins (often abbreviated as CD). These proteins are used for the identification and investigation of cell surface molecules present on leukocytes. CD molecules can act in numerous ways, often acting as receptors or ligands (the molecule that activates a receptor) important to the cell. A signal cascade is usually initiated, altering the behavior of the cell. Some CD proteins do not play a role in cell signaling, but have other functions, such as cell adhesion. Approximately, 320 CD cell surface proteins are known as of today which are involved in various physiological functions.

Monoclonal antibodies (mAbs) have been generated by different laboratories around the world against epitopes on the surface molecules of leukocytes and nucleated erythrocytes. Since then, its use has expanded to many other cell types, and more than 320 CD unique clusters and subclusters have been identified. The proposed surface molecule is assigned a CD number once two specific monoclonal antibodies (mAb) are shown to bind to the molecule. If the molecule has not been well-characterized, or has only one mAb, it is usually given the provisional indicator “w” (as in “CDw186”).

Anti-CD antibodies are also used as cell markers. For example, anti-CD34 antibody is used to capture and label embryonic cells, and anti-CD45 antibody for capturing and labeling leukocytes. The usefulness of these antibodies is obvious to anybody who is skillful biochemist or biologist. The table below illustrates the cell surface markers of some of the cells.

TABLE 3
CDs as Cell Markers

| Type of cell | CD markers |
|---|---|
| stem cells | CD34+, CD31− |
| all leukocyte groups | CD45+ |
| Granulocyte | CD45+, CD15+ |
| Monocyte | CD45+, CD14+ |
| T lymphocyte | CD45+, CD3+ |
| T helper cell | CD45+, CD3+, CD4+ |
| Cytotoxic T cell | CD45+, CD3+, CD8+ |
| B lymphocyte | CD45+, CD19+ or CD45+, |
| | CD20+ |
| Thrombocyte | CD45+, CD61+ |
| Natural killer cell | CD16+, CD56+, CD3− |

Camel and Shark Heavy Chain-Only Antibodies and their Analogs as Therapeutic Agents:

Their ability to enter cells and cross blood brain barrier (BBB) make camelid and shark heavy chain-only antibodies and their analogs unprecedented biomolecules to treat human and animal diseases, including solid tumors and neurodegenerative diseases which so far have not been treatable. In particular, micro-, subnano- and nano-antibodies (exemplary structures include 2-4 and their bivalent and multivalent constructs 5-8, and analogs) are potential therapeutic agents for the treatment of diseases, namely, viral, bacterial, cancer, neurological, cardiac, metabolic, and diseases of immune disorders. Specifically, all analogs with Val37, Gly44, Leu45 and Trp47 of Vab domain substituted by one of the hydrophilic amino-acids, namely, lysine, histidine, etc., containing few 1-10 hydrophilic amino acid residues from the hinge-region, because of their higher solubility and stability to low pH and proteases, constitute the most vital therapeutic agents which might be orally administrable.

Due to their ability to enter the cell and cross BBB, the camelid and shark antibodies and analogs represent an unprecedented class of biological molecules to detect and treat all human diseases, including, but not limiting to solid tumors, infectious diseases, diseases of brain, metabolic system and autoimmune disorders. Therapeutic applications of camelid and shark heavy chain-only antibodies and their analogs may include inhibition of cellular uptake of pathogens, for example, HIV by blocking the CCR5 and CXCR4 host co-receptor the virus uses for entry into the cell. These antibodies and their analogs may also be directed to interfere with the function of key proteins involved in the pathogenesis of the diseases. For example, HIV viral envelope proteins, namely vif, LEDGF/p75, TSA101, gp120 and gp41. In another example, the development of camelid and shark heavy chain-only antibodies and their analogs may be directed to inhibit/retard the function of a cancer causing proteins, for example, to inhibit the function of HER-2, a protein involved in the metastases of breast cancer. HER-2-positive breast cancer is a breast cancer that tests positive for a protein called human epidermal growth factor receptor-2 (HER-2), which promotes the growth of cancer cells. In about one of every three breast cancers, the cancer cells make an excess of HER2 due to a gene mutation. This gene mutation can occur in many types of cancer—not only breast cancer. Single-domain Herceptin might be more beneficial than the conventional Herceptin due to the fact that these antibodies are highly specific with none to low toxicity. Likewise, anti-Aβ42-nano-antibody, produced either in shark and/or camel, is likely to have superior performance to detect and treat Alzheimer's disease to classical mAbs.

Camelid and shark heavy-chain antibodies and analogs can be used against deadly toxin Clostridium botulinum (CB), a causative agent of botulism, against S. typhi, a causative agent of Typhoid fever, Bacillus anthracis a causative agent of anthrax, against Borrelia burgdorferi, a causative agent of Lyme disease, against Plasmodium falciparum, a causative agent of malaria.

### Improving the Therapeutic Efficacy of Existing Anti-Cancer Antibodies

The present invention describes the production and use of camelid and shark heavy chain-only antibodies and their analogs for improving the therapeutic efficacy of existing FDA approved therapeutic antibodies for the treatment of cancer and few other diseases. Because of their small size, low molecular weight, higher specificity, solubility, stability, bio-distribution, and higher binding affinity than the conventional antibodies, these single-domain antibodies are the embodiment of the present invention for pharmaceutical applications. For example, Herceptin, developed by Genentech, FDA approved, has become a major therapeutic option for patients with HER-2 positive metastatic breast cancer. Despite its dramatic benefits, cardiac toxicity remains a limiting factor for Herceptin's chemotherapy use. Because of the higher specificity of shark and camelid heavy chain only antibodies and their analogs in general, single-domain Herceptin will potentially have far fewer side effects than the conventional antibodies. Accordingly, this invention provides a method for producing antibodies such as Herceptin in camelids and shark for medical diagnostics and pharmaceuticals. Such antibodies include but not limited to the antibodies listed below:

Single-Domain Antibodies and Analogs for Improving Efficacy of the Existing FDA Approved Therapeutic Antibodies:

| HCAb | Therapeutic Application |
|---|---|
| Herceptin | Breast Cancer (HER-2 positive) |
| Zevalin | Non-Hodgkin Lymphoma |
| Carnpath | Chronic Lymphocytic Leukemia |
| Mylotarg | Acute Myeloid Leukemia |
| Rituxan | Non-Hodgkin Lymphoma |
| Gemtuzumab | Relapsed Acute Myeloid Leukemia |
| Alemtuzumab | B Cell Leukemia |
| Abciximab | Cardiac: Prevention of coagulation in |
| | coronary angioplasty |
| Crofab | Rattlesnake Antidote |
| Synagis | Respiratory Syncytial Virus |
| Remicade | Crohn's Disease, Rheumatoid Arthritis |
| Zenapax | Transplant rejection |

Other Therapeutic Applications of Camelid and Shark Heavy Chain-Only Antibodies and their Analogs

In still other embodiments, invention includes developing therapeutic camelid and shark heavy chain-only antibodies and their analogs that specifically binds to various disease biomarkers. Exemplary diseases include, but not limited to:

### Viral, Cardiac, Neurological, Bacterial (STDs and Anthrax), Autoimmune Disorders, Organ Transplant Rejection.

### Viral: HIV

HIV-1 entry is mediated by the binding of the viral envelope protein to a specific receptor, CD4, which is expressed on the cell surface of T-lymphocytes and certain monocyte/macrophage populations. However, in contrast to other retroviruses, HIV also requires other co-receptors, such as, CCR5 and CXCR4, for entry into cell. Heavy chain-only antibodies and their analogs against these receptors to prevent HIV-1 entry into the cell will be developed. Like, single-domain antibodies against the gp120 envelope protein of HIV and V3 loop and gp41 will be developed to neutralize HIV-1.

Camelid or shark can be immunized with the biomarkers associated with HIV. Heavy chain-only antibodies and their analogs that specifically bind to these biomarkers can be made using various methods such as, protease digestion, recombinant DNA technology and chemical means as described above. Heavy chain-only antibodies and their analogs that specifically bind to these biomarkers can be used in diagnosis, prognosis and/or treatment of HIV/AIDS. Exemplary HIV/AIDS biomarkers are shown below.

| | |
|---|---|
| CD4 binding site of gp120 | LEDGF/p75 |
| V3 loop of gp120 | TSA101 |
| gp41 | Viral Proteases |
| vif | Viral Reverse Transcriptase |
| CCR5 | Viral Integrases |
| CXCR4 | |

### HBV:

In yet other embodiment, the invention provides Heavy chain-only antibodies and their analogs for the treatment of chronic hepatitis B by developing the antibodies and their analogs against the surface antigens. As a precedent, conventional antibody against the surface antigen, OST-577, has resulted in reduction of HBV DNA by 75% in a small patient pool. Camelid and shark heavy chain-only antibodies and their analogs antibody against the surface antigens of HBV, including OST-577, will be evaluated against HBV.

### SARS:

SARS stands for severe acute respiratory syndrome. It is considered a bioterrorism weapon with mortality rates reaching over 40%. Fast and accurate diagnosis and treatment is of paramount importance to minimize causalities.

The SARS agent has been unambiguously identified as a new coronavirus member and named as SARS-coronavirus (SARS-CoV). Coronaviruses are enveloped, RNA viruses with the largest RNA genome known (About 30 kb). SARS-CoV pathogen causes fever, pulmonary edema, and diffuse alveolar damage in affected individuals leading to severe morbidity and mortality in humans.

The virus (represented as yellow sphere with spikes in the figure below) uses ACE2 receptor (shown as blue Y in the figure below) for internalization. Camelid or shark can be immunized with ACE2. Heavy chain-only antibodies and their analogs that specifically bind to ACE2 will be used to prevent SARS infection. In addition, Camelid or shark Heavy chain-only antibodies and their analogs will be developed against the SARS-CoV proteins, U122, S-protein and N-protein to therapeutically neutralize the virus.

Heavy chain-only antibodies and their analogs that specifically bind to these biomarkers can be used in diagnosis, prognosis and/or treatment of SARS.

Camelid and Shark Heavy Chain-Only Antibodies and their Analogs as Integrated Diagnostics and Therapeutics (Theranostics) Agents:

One of the embodiments of the invention is to tie the diagnostics with the therapeutics by using the same heavy-chain antibody for neutralizing the disease as used for its detection. In other words, the diagnostic biomarker is also the therapeutic target. Such an approach is only possible when the tracer molecule is cell and BBB permeable and is not toxic. EGFR is a epidermal growth factor receptor that is over expressed in several carcinomas, such as, carcinoma of lungs, breast, bladder, prostate, etc. Heavy-chain antibody and their analogs, specifically, radionuclide labeled nano-antibody against EGFR can be used to scan breast or lung cancer using a PET scanner. Let us assume that tissues in the lungs turned out to be positive, and biopsy confirmed the scanning results. Receptor-mediated endocytosis will internalize the heavy-chain antibody and whatever (radionuclide and toxins, e.g., ricin) attached to it, specifically the ones with lower molecular weight. Once internalized, the toxin or radionuclide attached to the HCAb will destroy the cell. Thus, the antibody used for tracing the disease will be the one to neutralize the disease. Table 4 below lists some examples of heavy-chain antibodies against the diagnostic biomarkers which can also serve as therapeutic targets (also known as theranostics).

TABLE 4
Integrated Diagnostic and Therapeutic Biomarkers

| Biomarker for Diagnostic | | | |
|---|---|---|---|
| and Therapeutic Target | | Disease | Reference |
| 1. | B7-H3 | Prostate Cancer | Am. J. Pathol., 167, 465 (2005) |
| 2. | CTLA-4 | Prostate Cancer | MSKCC.org/prostate cancer (Medarex, |
| | | | Inc., and |
| | | | Bristol-Meyer Squibb jointly |
| | | | developing a human anti-CTLA-4 |
| | | | (MDX-010) for the treatment of |
| | | | prostate cancer. |
| 3. | SSeCKS | Prostate Cancer | Defense Technical Info Center |
| | | | Annual Report, Jan. 3, 2000 |
| 4. | AMACR | Prostate Cancer | Cancer Res., 62, 4427 (2002) |
| 5. | TMPRSS2-ERG | Prostate Cancer | Science, 310, 644 (2005) |
| 6. | PCA3 | Prostate Cancer | Clin. Chem., 52, 1089 (2006) |
| 7. | EPCA-2 | Prostate Cancer | J. Urolology, 174, 514 (2005) |
| 8. | HEPSIN | Prostate Cancer | J. Urol., 171, 187 (2004) |
| 9. | BAG-1 | Prostate Cancer | The prostate, 45, 801 (2006) |
| 10. | HER-2 | Breast Cancer | Semin. Oncol., 28, 43 (2001) |
| 11. | Notch-4 | Breast Cancer | Stem Cell Rev., 3, 169 (2007) |
| 12. | ALDH-1 | Breast Cancer | Stem Cell, 1, 555 (2007) |
| 13. | CXCR4 | Breast Cancer | Cell Proliferation, 36, 59 (2003) |
| 14. | RS/DJ-1 | Breast Cancer | Clin. Cancer Res., 7, 3328 (2001) |
| 15. | EGFR | Colorectal Cancer | Cancer Res., 57, 4838 (1997) |
| 16. | SMAD4 | Colorectal Cancer | Clin. Cancer Res., 11, 2606 (2005) |
| 17. | KRAS & BRAF | Colorectal Cancer | Nature Rev., 9, 489 9 (2009) |
| 18. | p53, TS, MSI-H | Colorectal Cancer | 2009 Gastrointestinal Cancer Symp. |
| 19. | REG1A and EXTL3 | Colorectal Cancer | AACR Meeting Abs. Sep. 12, 2006 |
| 20. | TIMP-1 | Colorectal Cancer | Edwards et al. (Eds.), The Cancer |
| | | | Degradome, Springer Science, 2008 |
| 21. | P1K3CA | Colorectal Cancer | J. Clin. Oncology, 27, 1477 (2009) |
| 23. | VEGF | Colorectal Cancer | Korean J. Gastro., 53, 68 (2009) |
| 24. | HAAH | Colon Cancer | J. Biomed. Biotech. 4, 175 (2004) |
| 25. | EpCAM | Colorectal Cancer | Endoc. Related Cancer, 11, (2004) |
| 26. | gp120 | HIV/AIDS | Antiviral Res., 80, 251 92008) |
| 27. | Vif | HIV/AIDS | J. Virol., 66, 6489 (1992) |
| 28. | LEDGF/p75 | HIV/AIDS | J. Virol., 78, 9524 (2004) |
| 29. | TS101 | HIV/AIDS | Antiviral Res., 78, A18 (2004) |
| 30. | Ki67, Ras and EGFR | Lung | Eur. Respir. J., 19, 1151 (2002) |
| 31 | Bax and p16^{INK4A} | Lung | Eur. Respir. J. 19, 134 (2002) |
| 32. | Amyloid-beta | Alzheimer's | J. Clin. Invest., 115, 1121 (2005) |
| | | Disease (AD) | |
| 33. | Tau | AD | Neurology, 68, 1718 (2005) |
| 34. | DJ-1 | AD | Neurobiol. Dis., 28, 122 (2007) |
| 35. | Gamma-Secretase | AD | J. Clin. Psychiatry, 70, 281 (2009) |
| 36. | ALZAS | AD | J. Cell. Mol. Med., 12, 1094 (2008) |
| 37 | ApoE | AD | J. Cell. Mol. Med., 12, 1094 (2008) |
| 38. | Cystatain | AD | J. Alzheimer's Dis., 8, 377 (2005) |
| 39. | VGF Protein | AD | J. Alzheimer's Dis., 8, 377 (2005) |
| 40. | Apo A1 | AD | J. Alzheimer's Dis., 8, 377 (2005) |
| 41. | Kininogen Precursor | AD | J. Alzheimer's Dis., 8, 377 (2005) |
| 42. | Apo-H | Parkinson's | Neurology of Dis., xxx (2008) |
| | | Disease (PD) | |
| 43. | BDNF | PD | Neurology of Dis., xxx (2008) |
| 44. | IL-8 | PD | Neurology of Dis., xxx (2008) |
| 45. | VDBP | PD | Neurology of Dis., xxx (2008) |
| 46. | Beta2-Microglobulin | PD | Neurology of Dis., xxx (2008) |
| 47. | Alfa-Synuclein | PD | Neurology of Dis., xxx (2008) |
| 48. | TEM1 | Brain Tumor | PMID: 15624764 |
| 49. | PV-1 | Brain Tumor | PMID: 16278383 |
| 50. | EGFR | Multiple Cancers | Korean J. Gastro., 53, 68 (2009) |

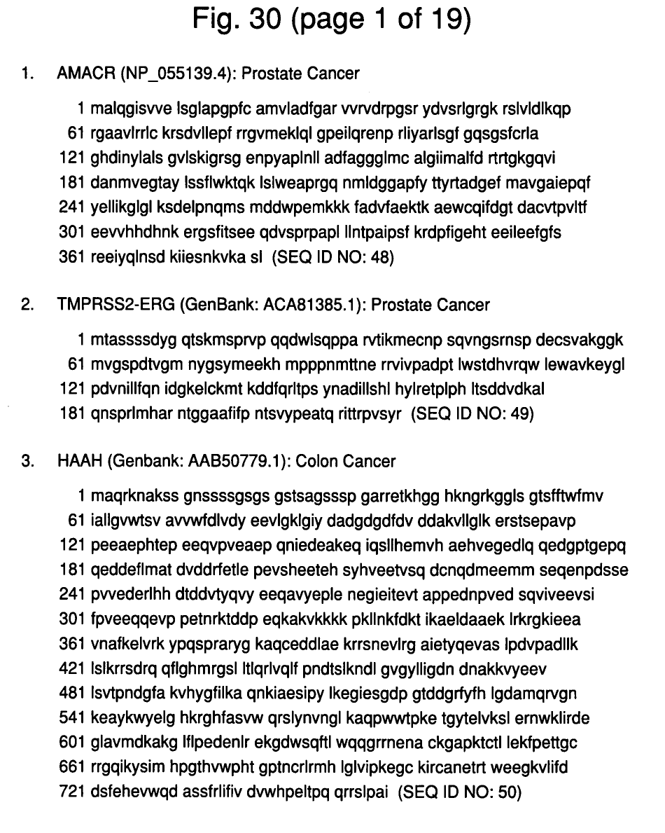

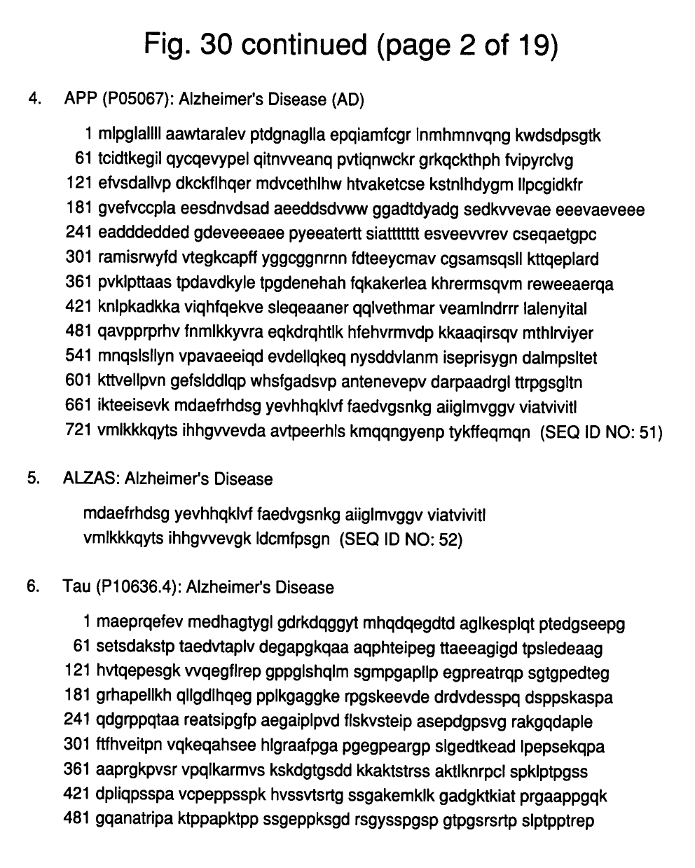

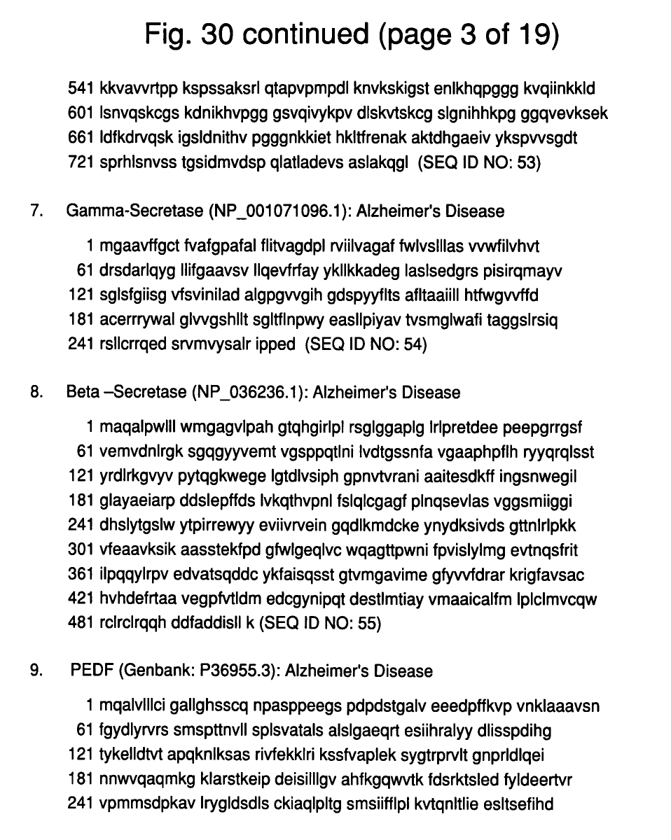

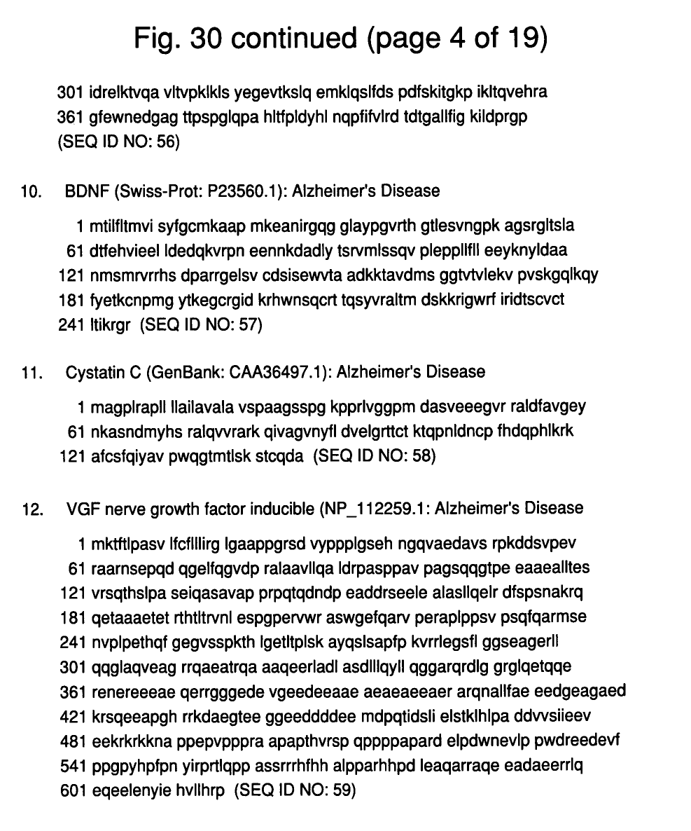

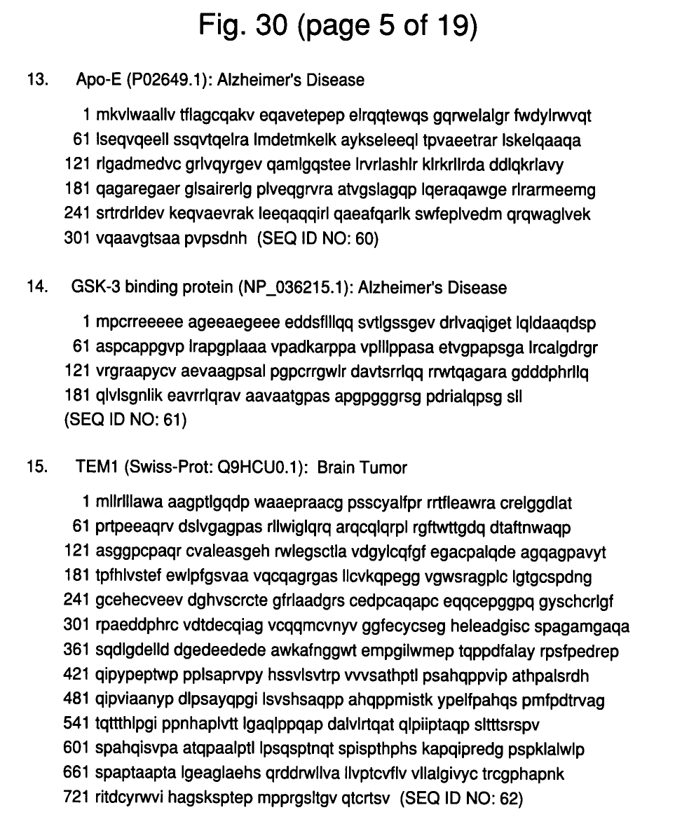

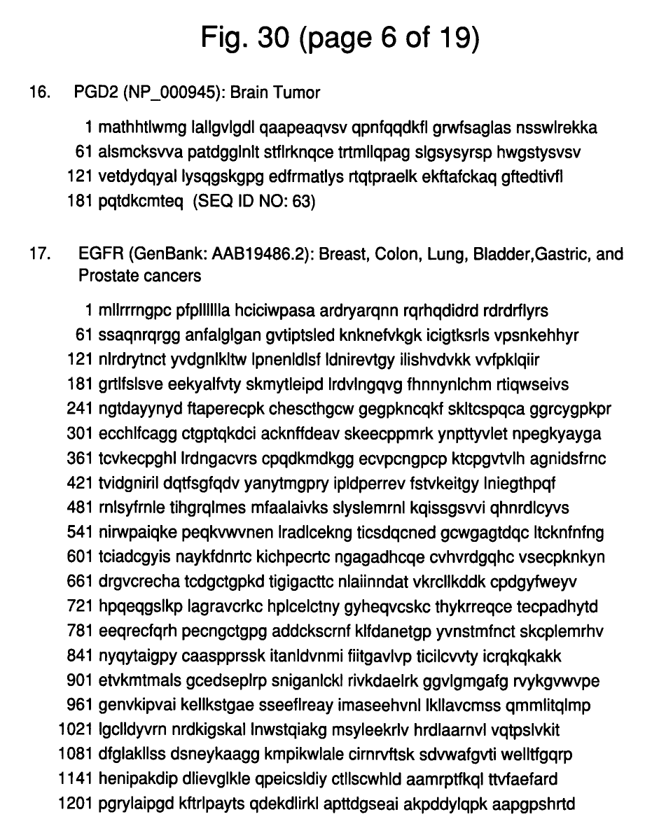

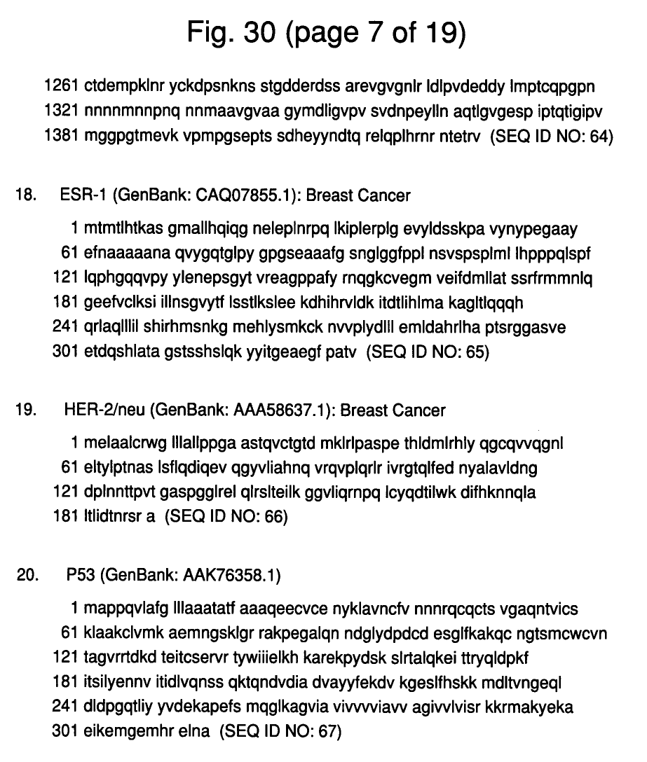

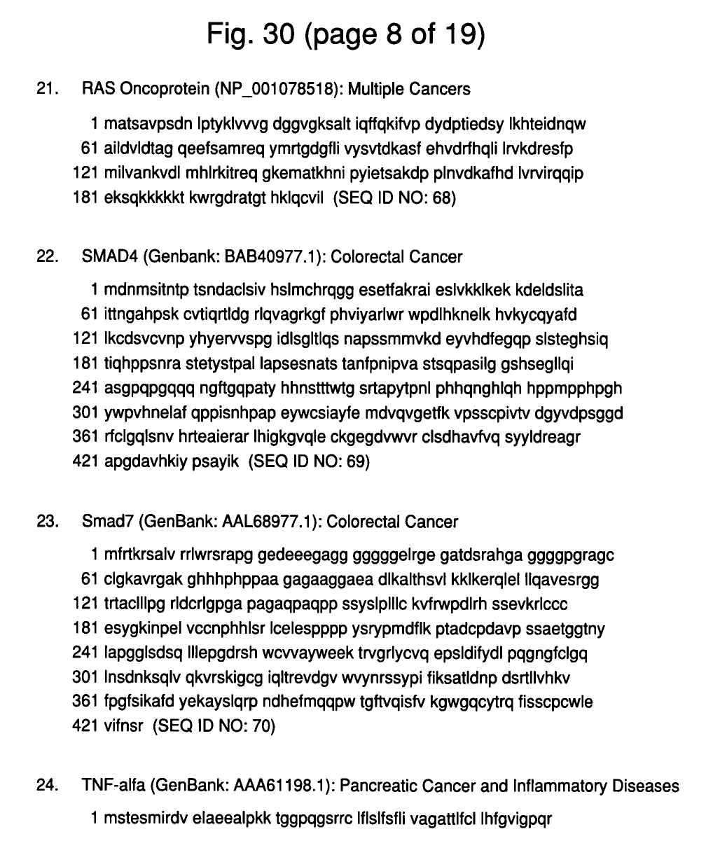

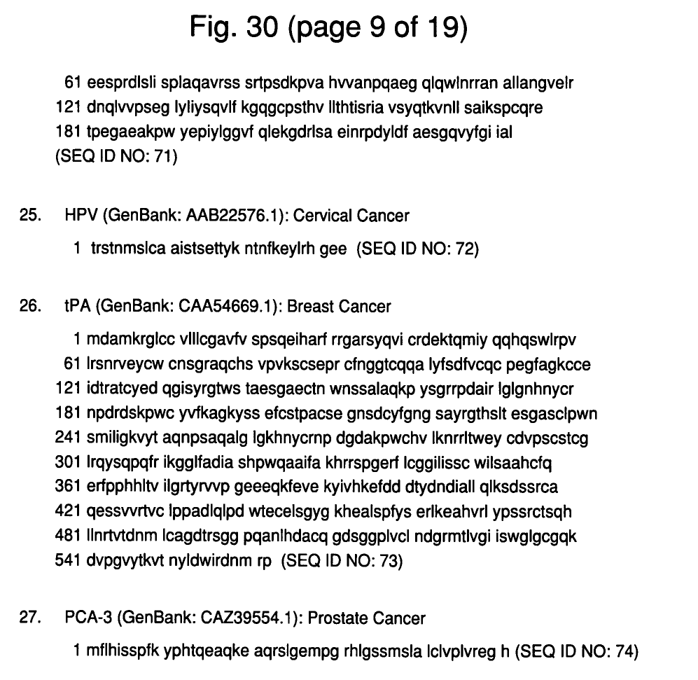

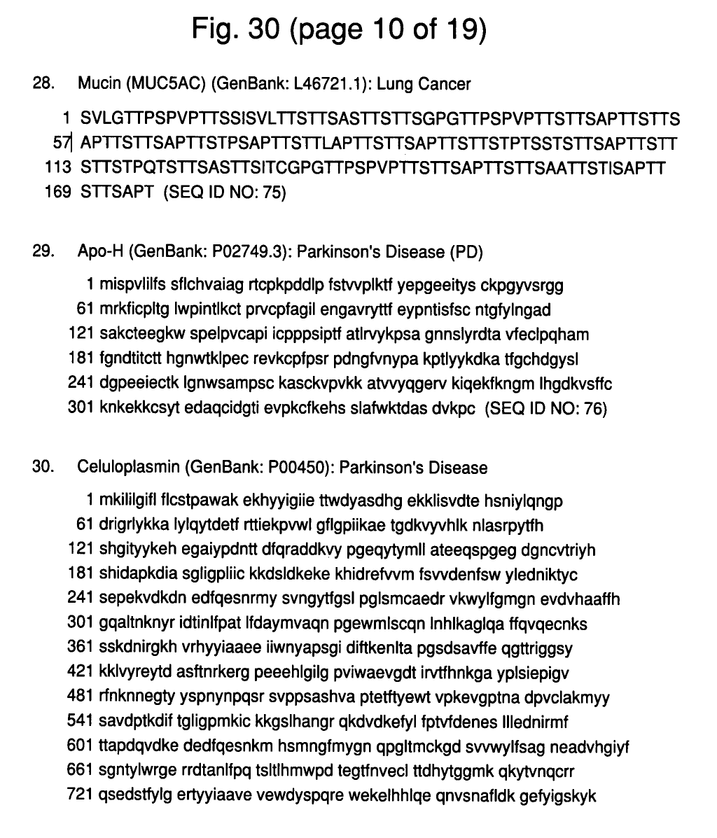

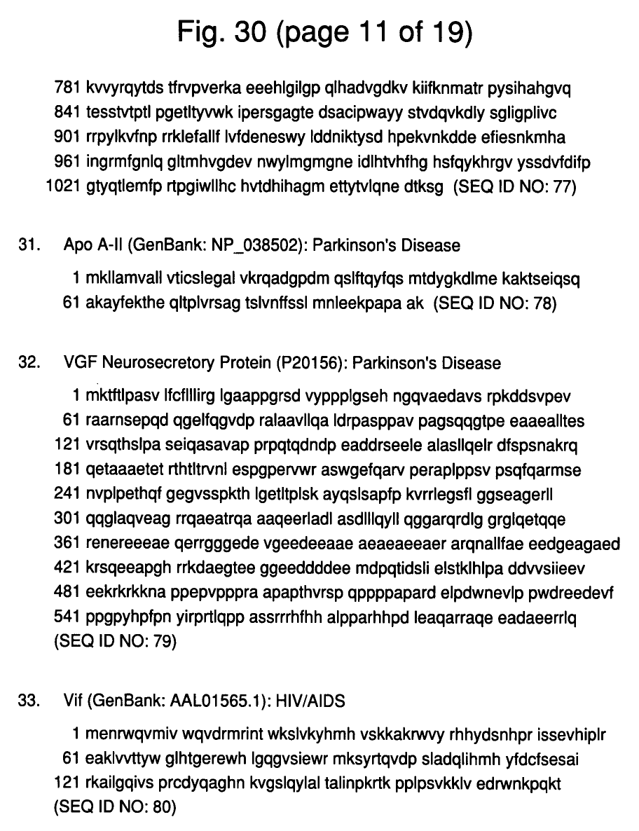

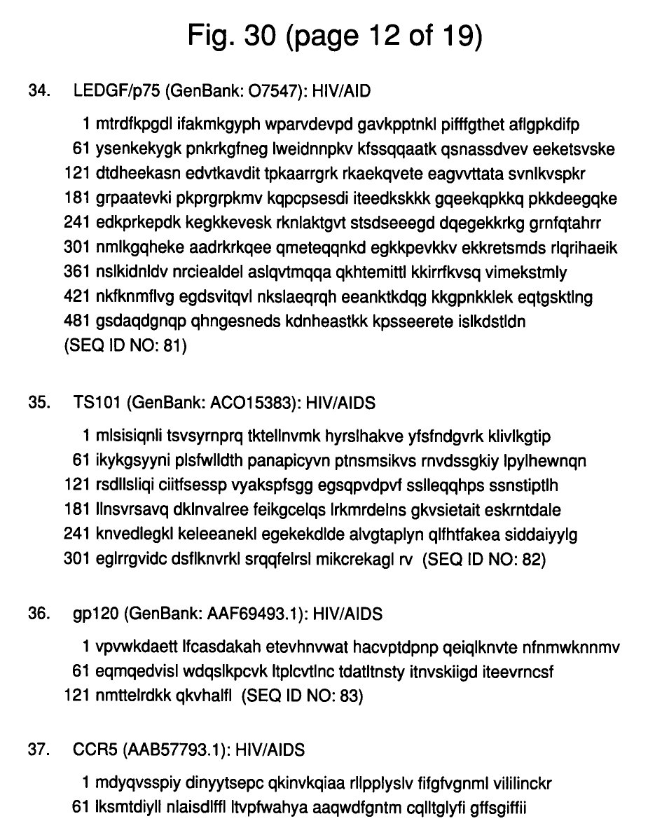

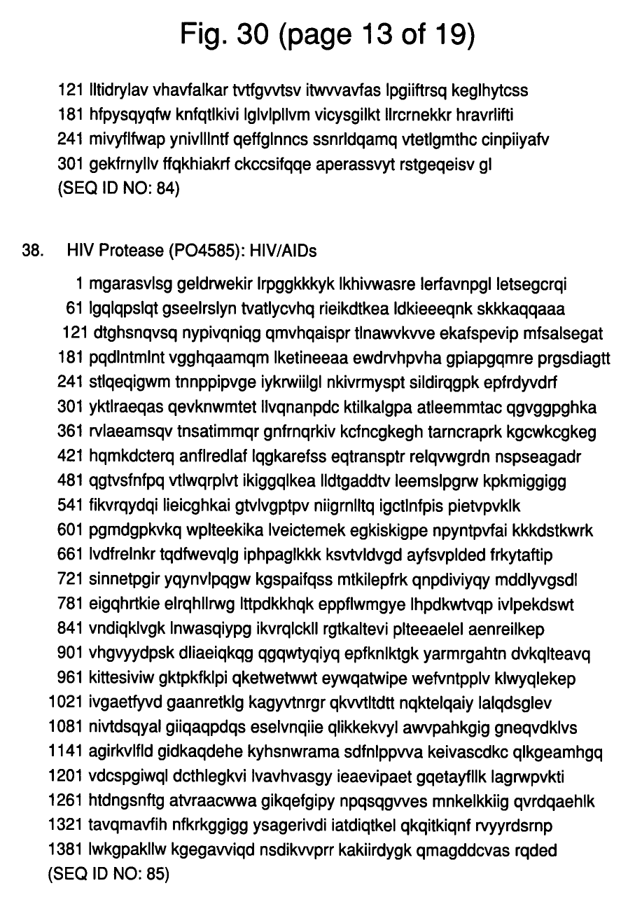

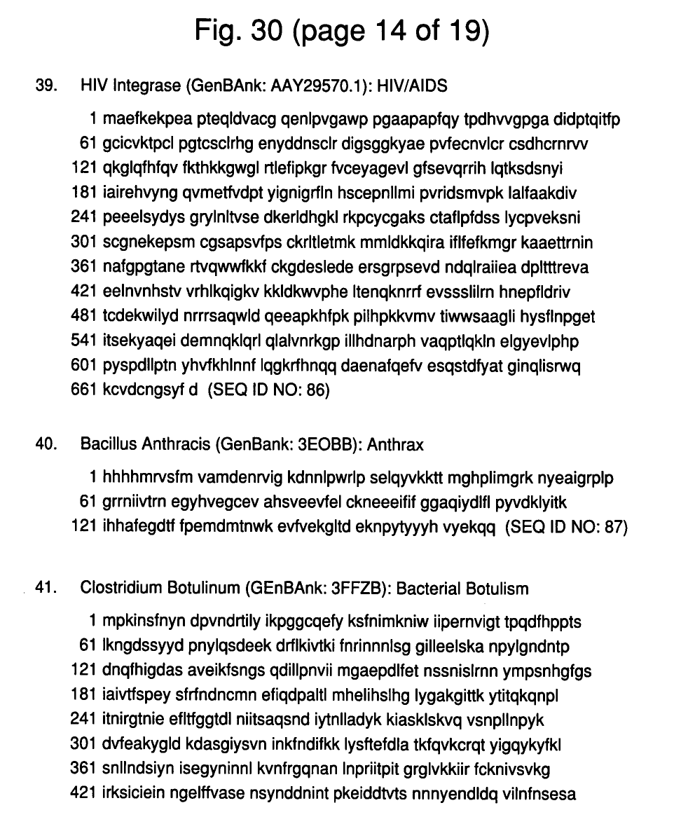

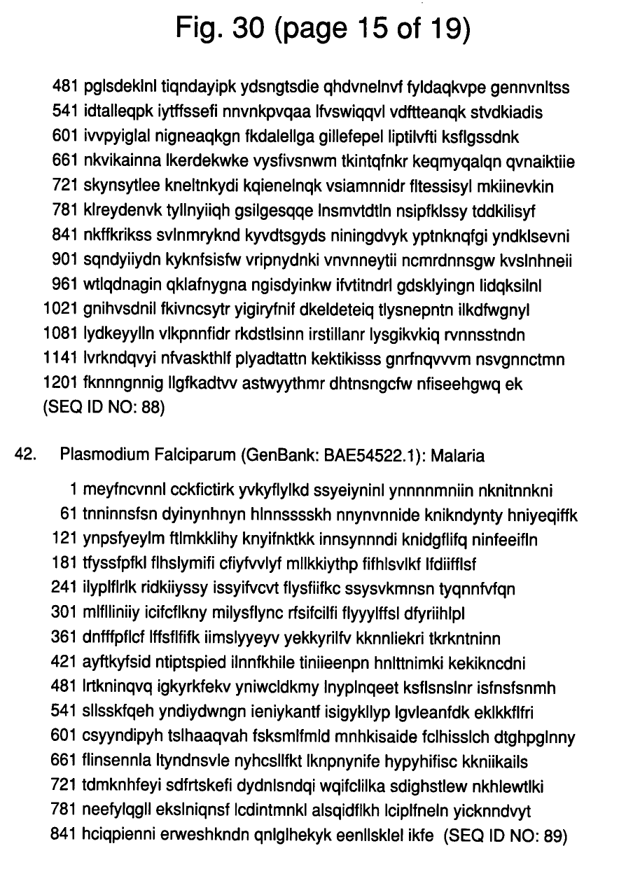

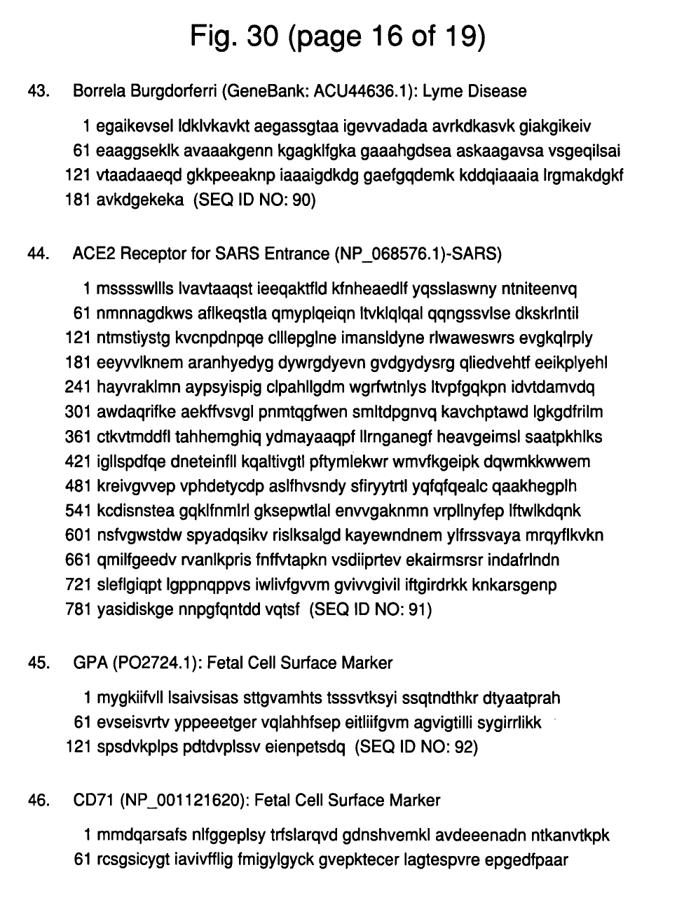

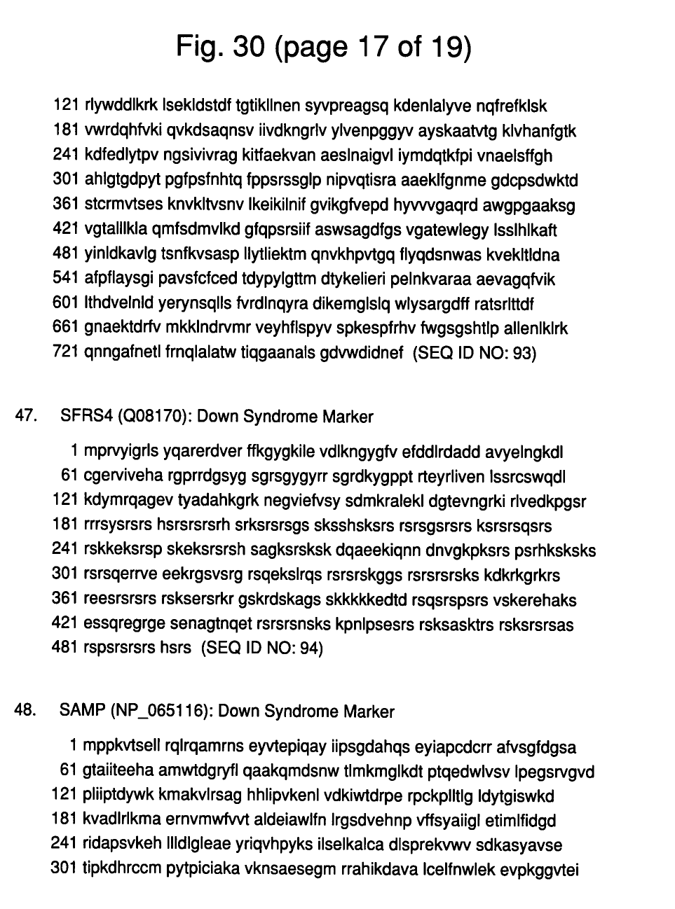

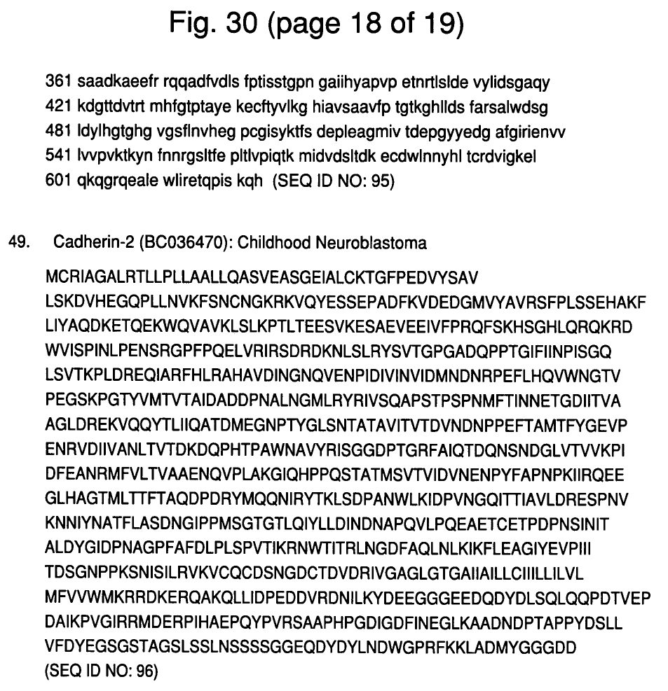

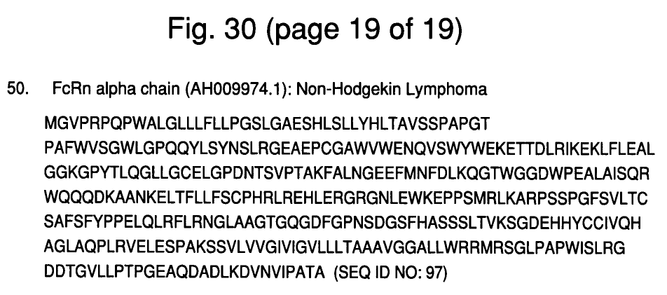

Amino acid sequences of the exemplary biomarkers are listed as SEQ ID NO: 48-97 and shown in FIG. 30.

### Nano-Antibodies as Drug Delivery Vehicles:

Most drugs, particularly macromolecules, including conventional monoclonal antibodies, cannot enter the cell and/or cross blood brain barrier (BBB) due to the presence of specialized endothelial tissue (aka BBB) between the brain and rest of the body. The delivery of therapeutic across the BBB is one of the biggest challenges of the pharmaceutical sciences in developing treatment for brain diseases. Currently, brain drug delivery approaches include both invasive and non-invasive procedures. Invasive procedures include cisternal, intracerebroventricular, and intracerebral injections as well as cell and tissue grafting. Non-invasive strategies take advantage of receptors that selectively express on brain endothelium that help mediate transcytosis of proteins essential for normal brain function across the BBB, including transferring, insulin-like growth factors (IGF), and low density lipoprotein [Broadwell R D, and Banks W A, In The Blood Brain Barrier (Pardridge W J., ed.), pp 156-199, Raven Press, New York]. Delivery of macromolecules to the brain can be achieved by coupling peptides, proteins, and nucleic acids to antibody “vectors' that bind to these transporters or receptors and undergo receptor-mediated transcytosis. Proof-of-principle for this approach has been obtained by using an anti-transferrin receptor antibody chemically coupled to peptides such as endorphin, VIP (vasoactive intestinal polypeptide), and brain-derived neurotrophic factor [Pardridge W M, Drug delivery to brain, J. Cereb. Blood Flow Metab., 17, 713 (1997)], or oligonucleotides and plasmid DNA [Boado R J et al., J. Pharm. Sci, 87, 1308 (1998)]. Unfortunately, only a small percentage of drugs gets delivered to the brain via receptor-mediated transcytosis across the BBB [Moos T, et al., Cell Mol. Neurobiol., 20, 77 (200______?)].

Nano-antibodies and their multivalent constructs against receptors on human cerebromicrovascular endothelial cells (HCECs) will constitute novel drug delivery vectors to transport drugs across the BBB. Because of their small sizes and high specificity, nano-antibodies (and their analogs) against the HCEC receptors, when covalently coupled to other drugs such as antibodies (Nano- and classical antibodies), gene delivery vectors, small-interfering RNA (Si-RNA), micro-RNA, antisense-oligonucleotides, enzymes, proteins, peptides, nano-particles, and lipids, etc, will serve as novel vehicles to deliver drugs across the BBB and solid tumors.

The present inventions also contemplate diagnostic systems in kit form. A diagnostic system of the present inventions may include a kit which contains, in an amount sufficient for at least one assay, any antibody or its analogs against an antigen, buffers in a packaging material. Typically, the kits will also include instructions recorded in a tangible form (e.g., contained on paper or an electronic medium) for using the packaged antibodies and their analogs in a detection assay for determining the presence or amount of an antigen in a sample.

The various components of the diagnostic systems may be provided in a variety of forms. For example, required enzymes, antibodies and their analogs may be provided as a lyophilized reagent. These lyophilized reagents may be pre-mixed before lyophilization so that when reconstituted they form a complete mixture with the proper ratio of each of the components ready for use in the assay. In addition, the diagnostic systems of the present inventions may contain a reconstitution reagent for reconstituting the lyophilized reagents of the kit.

Some preferred kits may further comprise to a solid support for anchoring the antibody or its analogs of interest on the solid support. Examples of such solid support include but are not limited to beads, microparticles (for example, gold and other nano particles), microarray, microwells, multiwell plates and microchannels. The solid surfaces may comprise a first member of a binding pair and the antibody or its analogs may comprise a second member of the binding pair. Binding of the binding pair members will anchor the antibody or its analogs to the solid surface. Examples of such binding pairs include but are not limited to biotin/streptavidin, hormone/receptor, ligand/receptor, antigen/antibody.

Typical packaging materials would include solid matrices such as glass, plastic, paper, foil, micro-particles and the like, capable of holding within fixed limits antibodies and their analogs of the present invention. Thus, for example, the packaging materials can include glass vials used to contain sub-milligram (e.g., picogram or nanogram) quantities of antibodies or their analogs or they can be microtiter plate wells to which antibodies or their analogs of the present inventions have been operatively affixed, i.e., linked so as to be capable of participating in detection method.

The instructions will typically indicate the reagents and/or concentrations of reagents and at least one assay method parameter which might be, for example, the relative amounts of reagents to use per amount of sample. In addition, such specifics as maintenance, time periods, temperature, and buffer conditions may also be included.

### Example 1

### Production of Parent Heavy-Chain Mini-Antibodies (HCmnAbs) of Structure 1

Host animals such as camel, llama, or alpaca will be immunized with the desired antigen(s), for example B7-H3, a biomarker for prostate cancer or Amyloid-beta peptide antigenic peptide for detecting amyloid plaque, following the procedures described by Murphy et al, in 1989 [Am. J. Vet. Res., 50, 1279 (1989)], but with slight modification. Typically, immunization of camels is done with 50-100 ug immunogen per injection but 250 ug or higher amount of peptide per injection will be used, followed by 4 booster shots every two weeks after the initial injection. For baby sharks, 10 ug antigen/injection will be used. One antigen per animal for immunization will be used, though it may be feasible to immunize an animal simultaneously with multiple antigens to raise an immune response to each antigen separately, which can make the production cost effective [EMBO, J., 17, 3512 (1998); J. Immunol. Methods, 240, 185 (2000)].

After immunization, 100 ml camel blood (or 5 ml from shark) will be withdrawn from the animal and the total IgGs will be precipitated out using ammonium sulfate precipitation procedure. Using size exclusion chromatography over Sephadex G-25, the conventional IgGs, MW 150 K Da, will be isolated from the mini-IgG with MW of 90 to 100 K Da. Affinity purification to obtain high affinity HCmnAb will be done by magnetic beads coated with antigenic peptide.

### Example 2

### Recombinant Production of Micro, Sub-Nano- and Nano-Antibodies 1a, 1b, and 1c

mRNA Isolation: Nano-antibody 1c will be produced by recombinant means which will involve using 10 ml blood from immunized camels, isolating total RNA from peripheral blood lymphocytes (PBLs). mRNA will then be isolated using Nucleotrap® mRNA kit. About 10 ug mRNA will be used for preparing first strand of cDNA after oligo (dT) priming using high fidelity reverse transcriptase.

cDNA Preparation: DNA fragments encoding nano-antibody 1c (Vab-hinge region) will be amplified by PCR using 1.0 ug cDNA, 80 to 100 pmol of Vab primer SEQ ID NO: 5 and hinge-region specific SEQ ID NO: 6, respectively, 0.2 mM dNTPs, 1 mM MgCl2, 5 ul of 10×PCR buffer, and 0.6 ul Taq DNA Polymerase. After a first denaturation round of 94° C. for 10 minutes, 35 to 36 cycles of amplification will be performed under conditions as described below:

| | |
|---|---|
| Denaturation: | 20 seconds at 94° C. |
| Annealing: | 30 Seconds at 56° C. |
| Extension: | 50 seconds at 72° C.. |
| Final Extension: | 10 min, 72° C. |

The sequence of the primers used for PCR amplification are as follows:

| | |
|---|---|
| 5′-gcaggagtctggaggaggc-3′ | (SEQ ID NO: 5) |
| 5′-ggacatctgggacacgtgcac-3′ | (SEQ ID NO: 6) |

Restriction sites such as Xho-1, Kpn-1, Barn-H1, Sac, etc., will be built in the forward primer that are compatible with the vector of choice for inserting the PCR amplified product in the vector. The PCR product will be sized and purified on 1.5% agarose gel. The PCR product of the nano-antibody 1c gene may be reamplified using 0.1 ug of the product using above conditions to improve the quality of the amplicon. The PCR amplicon will again be size selected and purified for restriction endonuclease digestion. Schematics of the cloning strategy is shown in FIG. 5.

### Example 3

### Library or Plasmid Construction

Prior to cloning the PCR amplicon encoding Vab-CH2-CH3 fragments of micro-antibody, vector and the amplicon DNA will be digested with Sfi1 and Not1 (Roche) following the cocktail:

| | Vab-CH2—CH3 | | Vector (pJT1) | |
|---|---|---|---|---|
| DNA | 5 | ug | 10 | ug |
| 10× Restriction Buffer | 5 | ul | 5 | ul |
| Sfi1 (10U/ul) | 8 | ul | 4 | ul |
| Water to | 50 | ul | 50 | ul |
| Incubate 50° C. for 8 hour | | | | |
| Not1 | 35 | U | 30 | U |
| Reaction Buffer | 4.5 | ul | 4.5 | ul |
| Water to | 60 | ul | 60 | ul |
| Incubate at 37° C. for 4-5 | | | | |
| hours. | | | | |
| Ethanol Precipitate at −70° C. | Pellet | | Pellet | |
| Water | 50 | ul | 50 | ul |

| | | |
|---|---|---|
| Agarose gel (1.5%) | Pure DNA | Pure Vector DNA |
| purification | Encoding micro-HCAb | |

Library Ligation

| | | |
|---|---|---|
| Vab-CH2—CH3 DNA = | 200 | ng |
| Vector DNA = | 1000 | ng |
| 10× Ligase Buffer = | 5 | ul |
| T4 DNA Ligase = | 10 | U |
| Water to = | 50 | ul |

The reaction mixture will be incubated for 15 hours at 4° C., followed by ethanol precipitate at −70° C. The pellet will be suspended in 10 ul. Phage Display Vectors used will be either pFARBER (NFCR) or pLUCK (Pharmacia) or pJT1 (Sigma)

### Electroporation

250 ul of E. Coli TG1 cells will be made electrocompetent with BRL Cell-Porator® following vendor protocol.

### Example 4

### Selection of Phage-Displayed Antibody Analog Library

Electroporated TG1 cells will be transfected with the vector-Vab-CH2-CH3 DNA or vector-Vab-HR-CH1 or vector-Vab-HR-CH2. Approximately, 10¹⁰ cells will be grown to mid-logarithmic phase before injection with M13K07 helper phages. Virions will be prepared as described in the literature [Andris-Widhopf J., et al, J. Immunology Methods, 242, 159 (2002)] and will be used for panning at a titre of 10¹³/ml. Specific Vab-CH2-CH3, or Vab-HR-CH1 or Vab-HR-CH2 antibody against the antigenic peptide will be enriched by five consecutive rounds of panning using magnetic beads conjugated with antigenic peptide. Bound phage particles will be eluted with 100 mM TEA (pH 10.00), and immediately neutralized with 1M Tris.HCl (pH 7.2) and will be used to reinfect exponentially growing E. Coli TG1 cells.

The enrichment of phage particles carrying antigen-specific Vab-CH2-CH3, or Vab-HR-CH1 or Vab-HR-CH2 will be assessed by ELISA before and after five rounds of panning. After the fifth panning, individual colonies will be picked up to analyze the presence of the virion binding by anti-M13-HRP conjugate.

### Example 5

### Phage-Displayed Micro-Antibody Library with Amino Acid 45 Substituted by Hydrophilic Amino acids

Phage-Displayed Micro-Antibody Library will also be developed to substitute amino acid at position 45 with Lys, His, Ser, Asn, Gln, Arg, Gln, Glu, Cys, Asp, Thr, and will be screened as described above. These changes will be made by substituting appropriate nucleotide codons in place of leu45 codons. This will help to study the structure-activity relationship.

### Example 6

### Expression and Purification of the Heavy-Chain Single Domain Nano-Antibody Fragments (Vab-HR-CH1 Human IgG)

The selected positive clones will be used to infect a new bacterial strain, HB 2151, a non-suppressor strain that recognizes the amber codon as a stop codon for soluble protein production. The HB2151 cell harboring the recombinant phagemids will be grown at 28° C. in 250 ml 2×YT-ampicillin, 1% glucose in culture flasks until OD₆₀₀ 0.7. The cells will be washed and resuspended in 250 ml 2×YT-ampicillin, supplemented with 1 mM isopropyl-BD-thiogalactopyranoside (IPTG), and will be incubated over night at 22° C. to induce protein expression. Before adding IPTG to the cultures, a portion will be spotted on an LB/ampicillin plate for future analysis of the clones. The culture will be then be centrifuged at 4000 RPM for 15 minutes to pellet the bacterial cells. The culture supernatant will then be screened by ELISA for antigen-specific binding.

### Example 7

### Recombinant Production of Micro-Antibody 1a, and Sub-Nano-Antibody 1b

The procedure will be similar to the one described above for micro-nano-antibody except the PCR primers will be different. Briefly, mRNA will be isolated from peripheral blood mononuclear cells (PBMs) and cDNA will be cloned by reverse transcriptase-PCR with oligo-dT promoter. Vab gene carrying the hinge region and CH2 will be amplified with PCR primers SEQ ID NO: 5 and 98 specific for amplifying Vab and CH2 domain to obtain amplicon encoding for sub-nano-antibody Vab-HR-CH2, 1b.

| | |
|---|---|
| Forward primer: | |
| 5′-gcaggagtctggaggaggc-3′ | (SEQ ID NO: 5) |
| Reverse primer: | |
| 5′-ggtccagctgctcgagtctgg-3′ | (SEQ ID NO: 98) |

To amplify the DNA for micro-antibody 1a, primers pairs SEQ ID NO: 5 and 99 will be used to produce PCR product encoding the Vab-CH2-CH3 gene

| | |
|---|---|
| Forward primer: | |
| 5′-gcaggagtctggaggaggc-3′ | (SEQ ID NO: 5) |
| Reverse primer: | |
| 5′-tagtgcaccaccatcaccatcactaa-3′ | (SEQ ID NO: 99) |

The PCR products will be purified on agarose gel, reamplified using nested PCR primers containing restriction site on both ends of the gene. The final PCR product will be cloned into the phagemid vector and transformed in electro-competent E. coli TGI cells. The sub-nano Vab repertoire of structure 3 will be expressed on phage after infection with M13K07 helper phages. Specific virions against the antigen will be enriched by three consecutive rounds of in-vitro selection on 96-well plates coated with antigen. The bound virions from antigen coated plated will be eluted with 0.1M Tris.HCl, pH 7.4, and will be used to infect exponentially growing E. coli cells. After three round of panning, polyclonal phage ELISA will be performed to monitor the success of selection. Pools of virions from each round will be incubated on antigen coated and non-coated wells. Binding will be detected using an anti-M13-HRP conjugate. Monoclonal phage ELISA will be used to identify individual positive clones, which will be then resequenced to identify Vab-Henge-CH2 gene. This gene will then be cloned and expressed in the periplasm. Purification of Vab-Hinge-CH2, 3, sub-nano-antibody will be done by affinity chromatography. The antibody will then be tested for recognition in ELISA.

Procedure for the production of Nano-antibody without the human CH1 Domain will be the same except the human CH1 domain will not be ligated to the PCR amplified Vab-HR DNA.

### Example 8

### Expression and Purification of the Heavy-Chain Single-Domain Micro-Antibody, 1a, Fragments and Analogs

The selected positive clones of Vab-CH2-CH3, Vab-HR, Vab-HR-CH1 with substituted amino acid at position 45, Vab-CH2-CH3 with substituted amino acid at position 45, Vab-HR with substituted amino acid at position 45 will be used to infect a new bacterial strain, HB 2151, a non-suppressor strain that recognizes the amber codon as a stop codon for soluble protein production. The HB2151 cell harboring the recombinant phagemids will be grown at 28° C. in 250 ml 2×YT-ampicillin, 1% glucose in culture flasks until OD₆₀₀ 0.7. The cells will be washed and resuspended in 250 ml 2×YT-ampicillin, supplemented with 1 mM isopropyl-BD-thiogalactopyranoside (IPTG), and incubated over night at 22° C. to induce protein expression. Before adding IPTG to the cultures, a portion was spotted on an LB/ampicillin plate for future analysis of the clones. The culture will be then be centrifuged at 4000 RPM for 15 minutes to pellet the bacterial cells. The culture supernatant will then be screened by ELISA for antigen-specific binding.

### Example 9

### Production of Bivalent Nano-Antibody (Vab-CH1-CH1-Vab) using Recombinant DNA Technology

The recombinant nano-antibody, Vab-CH1, from example 6 will be digested with restriction nucleaseNco1 to obtain

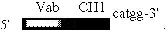

The restriction site Nco1 will be built in the PCR primers.

Meanwhile, the gene encoding the CH1-Vab (human CH1-camelid Vab) will be constructed and digested with Nco1, the restriction site for which is built at the 5′-end of PCR primer spanning the CHI of human IgG to obtain

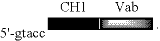

The ligation of these two gene constructs will give rise to Vab-CH1-CH1-Vab where Vab represents variable antigen-binding domain of camelid and or shark antibodies. Amplification of this bivalent construct will be done using PCR primers SEQ ID NO:5 and SEQ ID NO: 100.

| | |
|---|---|
| Forward primer: | |
| 5′-gcaggagtctggaggaggc-3′ | (SEQ ID NO: 5) |
| Reverse primer: | |
| 5′-tgcctaacggcgtgtcattatg-3′ | (SEQ ID NO: 100) |

The PCR product will be gel purified and inserted into the phagemid as described using restriction sites built in at both extremities of the amplified fragment as shown in FIG. 7.

Finally, the recombinant plasmid and the PCR fragment will be mixed and ligated using T4 DNA ligase. The ligated product, containing the bivalent nano-antibody gene construct will be used to transfect E. Coli WK6 electrocompetent cells. The clones containing the bivalent construct were screened by PCR using forward and reverse universal primers. Clone that will give rise to an amplified product of ˜1000 by will be sequenced using HCABI Prism 677) to confirm the bivalent gene insert. After expression, the encoded protein will be tested by ELISA.

### Example 10

### Chemical Synthesis of Analogs of Single-Domain Camel Antibodies

### Heavy-Chain Micro-Antibody 1a:

Micro-antibody 1a will be prepared by treating mini-antibody 1 (2 mg) with 1.0 ml of 10 mM TCEP (tris-carboxyethyl-phosphine) in 20 mM Phosphate/150 mM NaCl, pH7.4 at room temperature (RT) for one hour. The resulting micro-antibody 1a will be desalted on centricon-3 to remove the excess reagent and the buffer and stored at 4° C. in 1×PBS.

### Heavy-Chain Sub-Nano-Antibody 1b:

Micro-antibody 1a will be treated with trypsin or pepsin under controlled conditions to cleave the CH2-CH3 domains from the antibody. After deactivation of the proteolytic enzyme with fetal calf serum, the subnano-antibody 1b will be isolated using size exclusion chromatography.

### Heavy-Chain Nano-Antibody 1c:

Sub-nano-antibody 1b will be treated with pepsin at a low pH of 4.5 in 2M sodium acetate buffer under mild conditions for 1-8 hours to cleave the CH2-CH3 domains from the antibody. After deactivation of the proteolytic enzyme with fetal calf serum, the nano-antibody 1c will be isolated using size exclusion chromatography.

### Bivalent Nano-Antibody 1d:

Nano-antibody 1c will be treated with NHS-PEG-Mal, 17, in 50 mM MOPS/150 mM NaCl, pH 6.8, at RT for 1 hour to obtain the pegylated conjugate 11 (FIG. 8) which will be purified by dialysis on C-3 Amicon filters. Also, nano-antibody 1c will be modified with Traust's Reagent following manufacturer protocol to obtain thiolated nano-antibody 12. Conjugation of 11 with 12 in pH 6.5 buffer for 2 hours at RT will give, after dialysis on C-3 Centricon membranes, the dimeric conjugate 1d which will then be analyzed by ELISA and Western blot assays. An exemplary reaction schematics is shown in FIG. 8.

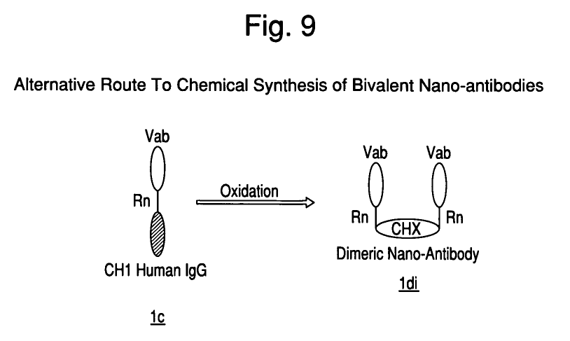

Alternatively, dimer 1d will be obtained by oxidation with iodine or sodium periodate as shown in below in FIG. 5. Briefly, the sub-nano-antibody, Vab-CH1, 1c, (1 mg), will be dissolved in 1.0 ml of 20 mM phosphate/NaCl, pH 7.4 (PBS), and treated with a solution of 10 mM sodium periodate in 90% PBS/10% DMF for 2 hours. The dimer 1d obtained will then be purified by size exclusion chromatography. An exemplary reaction schematics is shown in FIG. 9.

### Multivalent Analogs

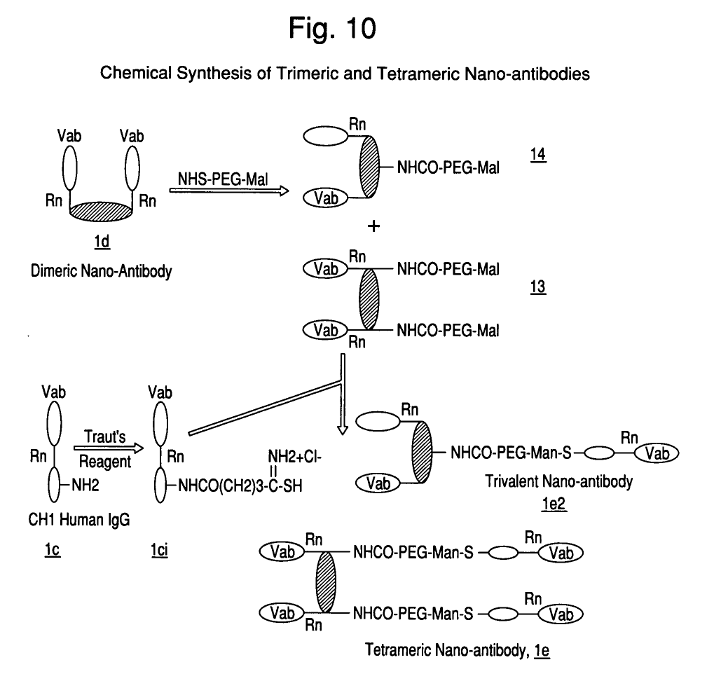

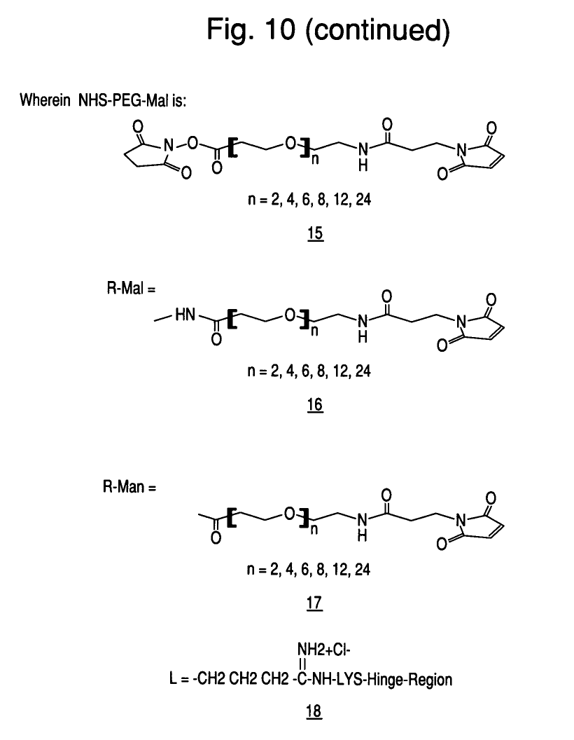

Chemical synthesis of trivalent, tetravalent and multivalent analogs of Vab domain of camelid antibodies lacking the light-chains will be developed as shown below in FIGS. 10, 11, 24.

### Protocol for Developing Trivalent and Tetravalent Vab Antibodies:

Bivalent Vab antibodies, 1d, prepared by oxidative dimerization or chemical ligation, will be conjugated with NHS-(PEG)₃-Mal (10 folds excess) in MOPS buffer at pH 7.0 for 1 hour at RT. Chemical ligation of the resulting dimeric and monomeric pegylated products 13 and 14 will with thiolated nano-antibody Vab-HR, 1c.i (FIG. 3) will be carried out by combining the two at pH 6.8 buffer containing 5 mM EDTA and allowing the reaction to occur at RT for at least 2 hours. The so formed trivalent, 1ei, and tetravalent Vab, 1e, constructs will be purified by size exclusion chromatography and stored at 4° C. in PBS containing 0.02% NaN₃.

Pentavalent and higher analogs of nano-antibodies (Vab domains of camel antibodies) can be similarly prepared.

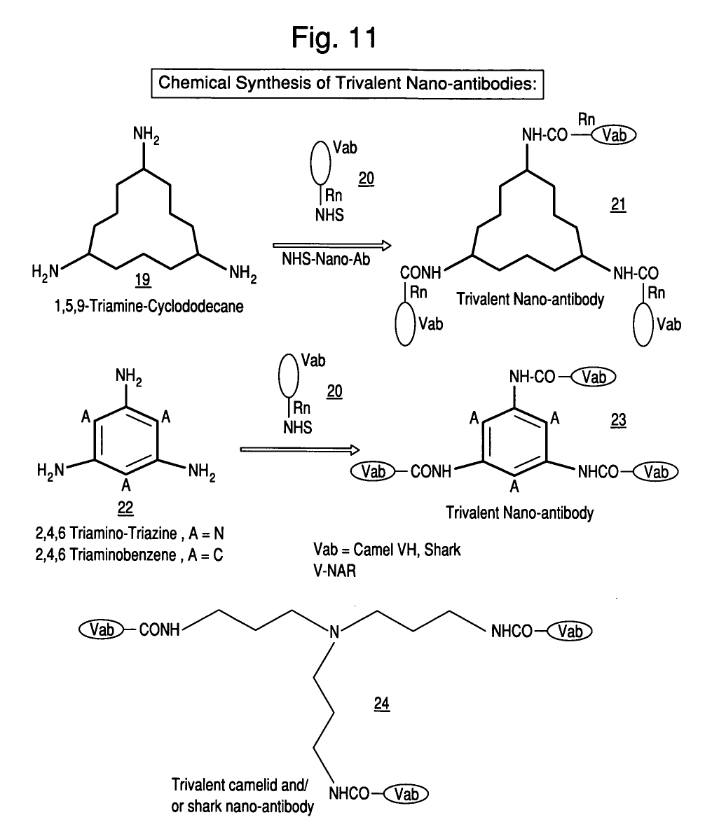

Trivalent nano-antibodies can also be readily prepared from symmetrical cyclic or acylic triamines, such as, 1, 5, 9-triamine-cyclododecane 19, or triazine like triamines of structure 22, or tris-isopropyl-amine like derivatives, 24, by treatment of the starting amine with n-hydroxy-succinimide (NHS)-analogs of nano-antibodies, such as 20 (FIG. 11). Purification can be accomplished by HPLC on a C4 or C8 column.

### Example 11

### Conjugates of Analogs of Single-Domain Camelid Antibodies

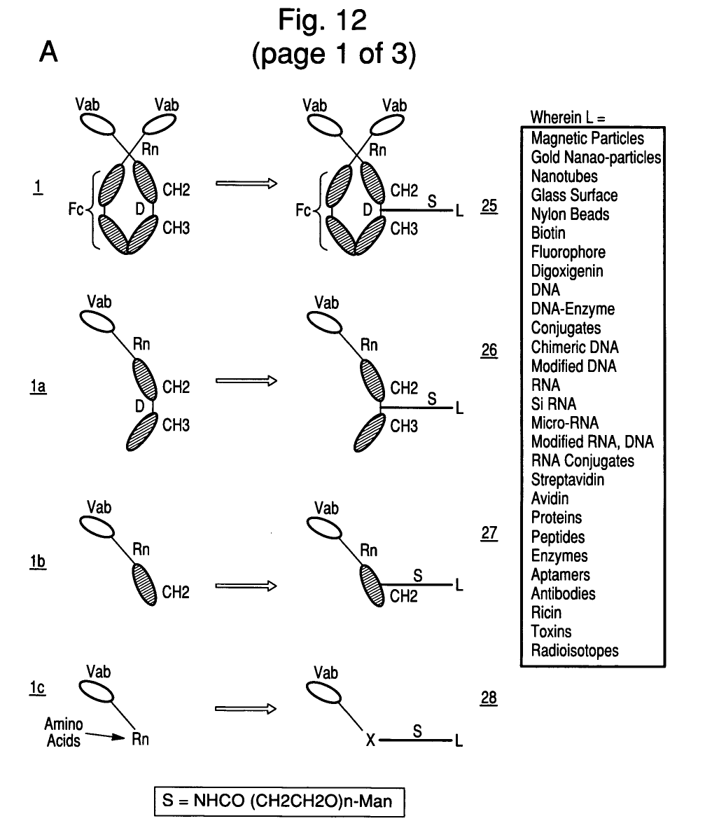

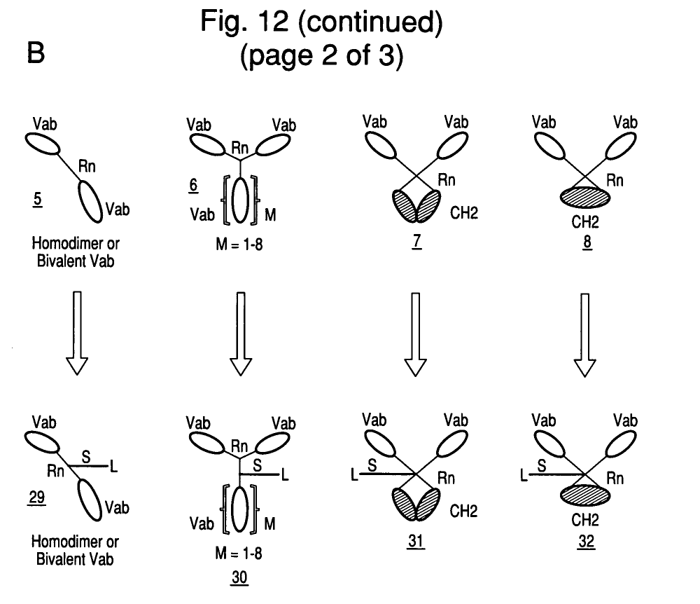

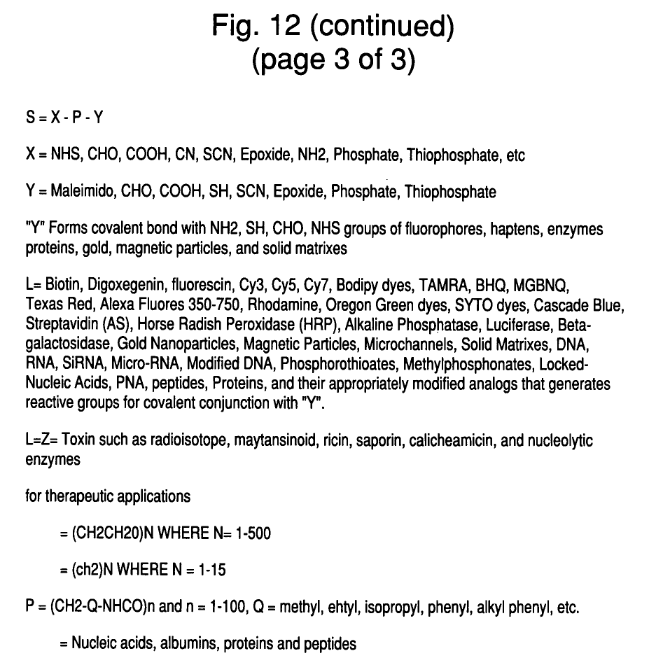

Conjugates of analogs of single-domain camelid antibodies can be prepared for diagnostic and therapeutic purposes. Examples of some conjugates are shown in FIG. 12.

Heavy-chain antibodies, 1, 1a, 1b, 1c, (1 mg each, approximately 22, 44, 66, 132 nmols, respectively) in 1.0 ml PBS, pH 7.0, will be treated with freshly prepared commercial.

10 mM NHS-PEG-Maleimide at room temperature for one hour with gentle rocking of the reaction mixture. After neutralizing the excess reagent with 10 ul of 0.1 M glycine, pH 7.0. The reaction mixture will be desalted by dialysis on centricon-10 to remove the hydrolysed reagent and excess glycine. The corresponding pegylated antibodies will then be conjugated with thiolated haptens, fluorophores, enzymes and proteins to obtain conjugates of haptens, proteins, enzymes and antibodies of structures 25, 26, 27, and 28, respectively (FIG. 12A). After desalting as described above, the conjugate can be further purified by affinity purification if needed.

Similarly, bivalent and trivalent constructs of nano-antibodies will be first pegylated and subsequently treated with thiolated haptens, fluorophores, enzymes and proteins to obtain their corresponding hapten, fluorophores, enzymes and protein conjugates of structures 29, 30, 31, and 32, respectively (FIG. 12B).

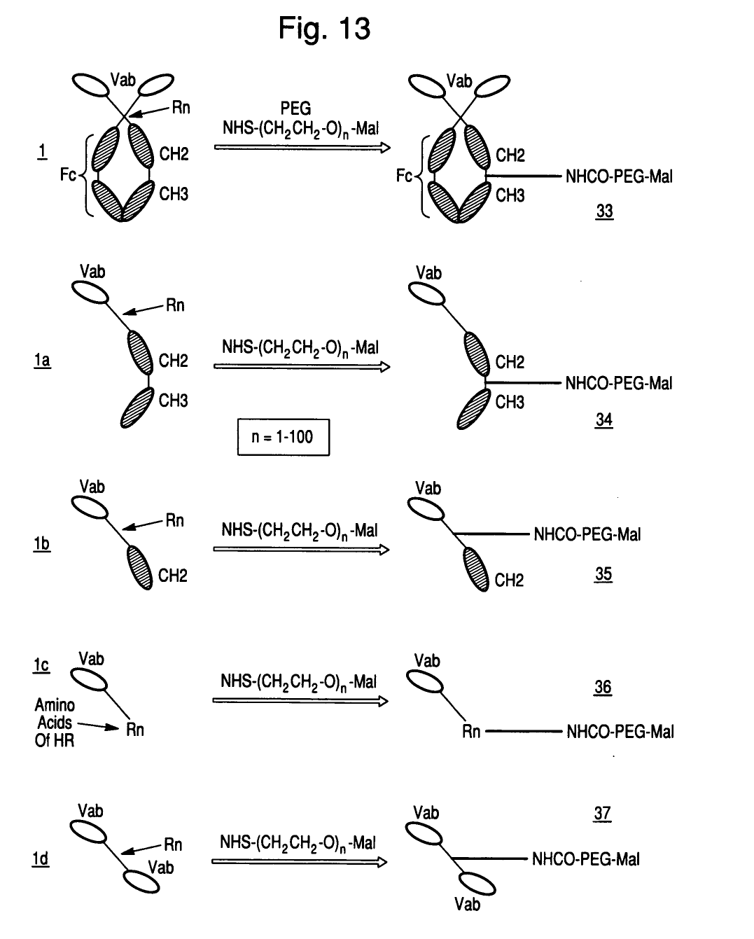

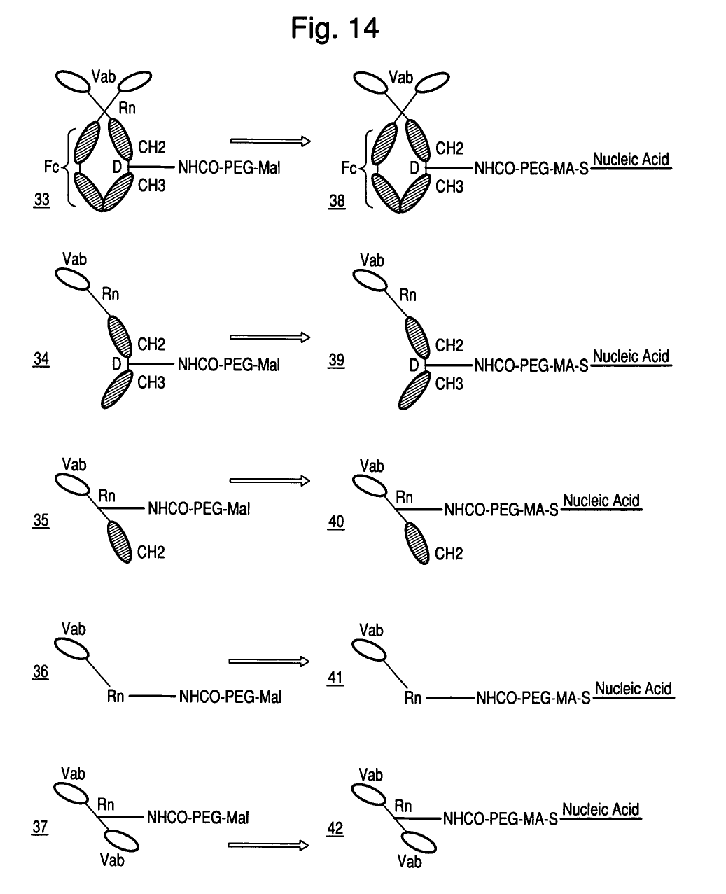

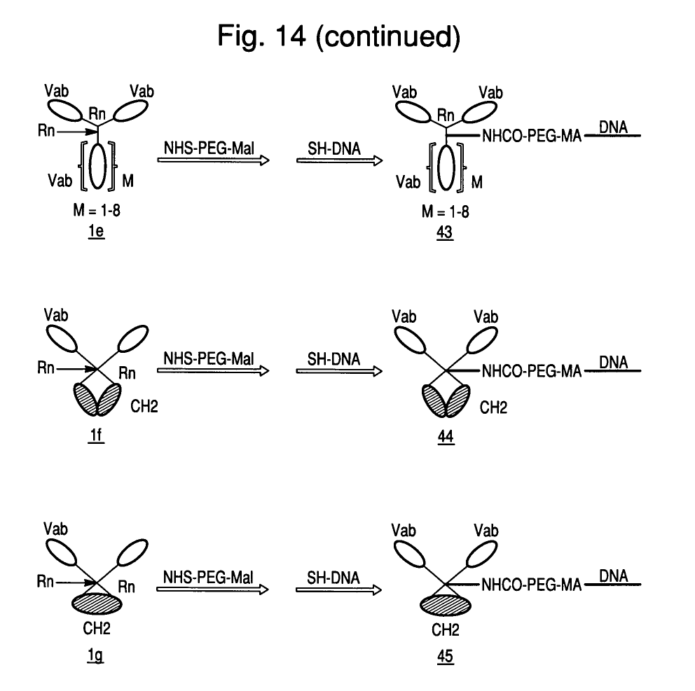

Nucleic acid conjugates of mini- and nano-antibodies 1a-1g will also be prepared using their pegylated conjugates followed by treatment with the thiolated-DNA/RNA molecules of interest. FIG. 13 shows the pegylation of mini-, micro-, sub-nano- and nano-antibodies to yield the corresponding pegylated products 33, 34, 35, 36, and 37, respectively as shown in FIG. 13. Though not shown, pegylation of 1e, 1f, and 1g will be performed as described above. FIG. 14 shows the conjugation of thiolated DNA/RNA (synthetic, recombinant or natural) to obtain mini- and nano-antibodies conjugated with DNA/RNA (nucleic acids) with the structures 38, 39, 40, 41, 42, 43, 44, and 45.

### Example 12

### Immobilization of Camelid Single-Domain Antibodies and Analogs onto Solid Matrixes

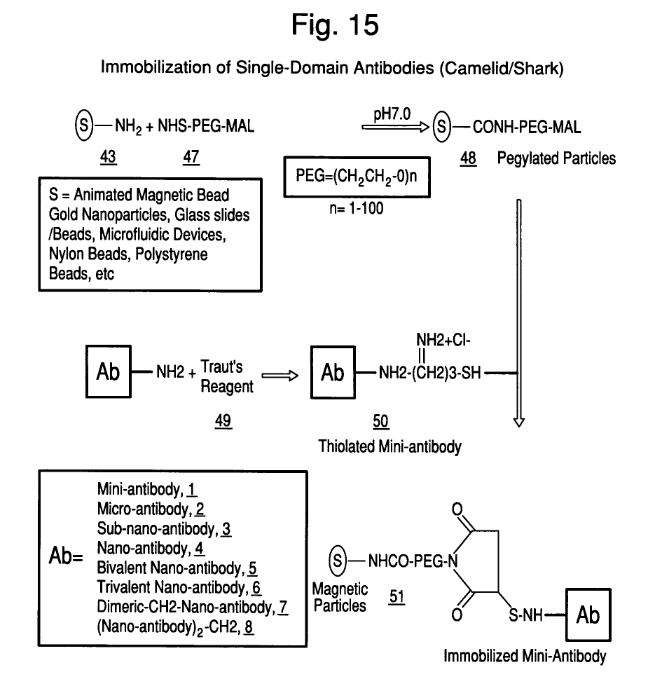

Immobilization of heavy-chain only antibodies, 1a through 1g, onto solid matrixes, such as gold particles, magnetic particles, microchannels, glass particles and other solid surfaces will be accomplished using the steps outlined in FIG. 15. Aminated solid matrix 46 will first be derivatized with NHS-PEGₙ-Mal 47, where n=1-100, (20 fold molar excess) at pH 7.0 for 1 hour at RT. The solid matrix will then be washed thoroughly with the same buffer (50 mM MOPS/150 mM NaCl, pH 7.0). Any unconjugated amine groups will be masked with sulfo-NHS-Acetate (Pierce) by incubated the solid matrix with 40 fold excess of the reagent at Ph 7.0 for 30 minutes. After washing off the excess masking reagent, the pegylated matrix 48 will then be conjugated with thiolated heavy-chain antibody 50 (2.5× excess) over the starting amine concentration. The conjugation will be performed at pH 6.5 for 2 hours at RT with gentle shaking of the matrix. The unused antibody will be recovered, and the matrix very well washed with 1×PBS/0.5% Tween-20 to obtain complex 51 in which nano-antibody is covalently bound to a solid matrix. The activity of the bound heavy-chain antibody will be measured using ELISA.

Direct labeling of heavy-chain antibodies with ligands and fluorophores can also be accomplished by treating the corresponding NHS analog of the hapten, such as, NHS-X-R, with the heavy-chain nano-antibody at neutral pH as described Above. Ligand can be aminated or thiolated or carboxylated. Anyone who is skilled in the art would know how to conjugate bifunctional linkers with amines, carboxyls and thiols.

### Example 13

### Production of Heavy-Chain Single-Domain Shark IgNAR (Structure 2)

Immunization of Sharks and Isolation of Shark IgNAR: Baby sharks will be immunized with the desired antigen(s), for example ALZAS, Tau, Abeta42 peptide which are the potential biomarkers for Alzheimer's disease, following the protocol described by Suran et al [J. Immunology, 99, 679 (1967)]. Briefly, the antigen (20 ug per kg animal weight), dissolved in 20 mg/ml keyhole limpet hemocyanin (KLH) supplemented with 4 mg/ml complete Freund's adjuvant, will be injected intramuscularly. Four booster shots every two weeks four weeks after the initial injection will be administered.

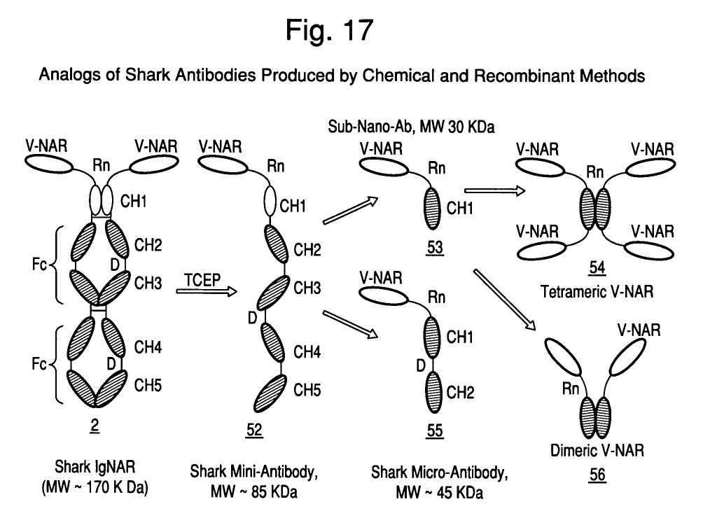

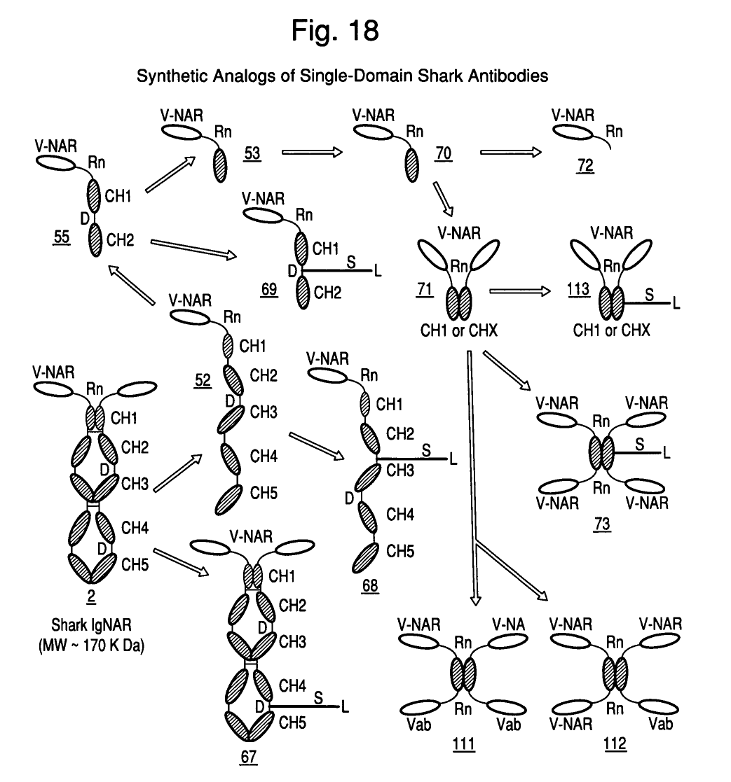

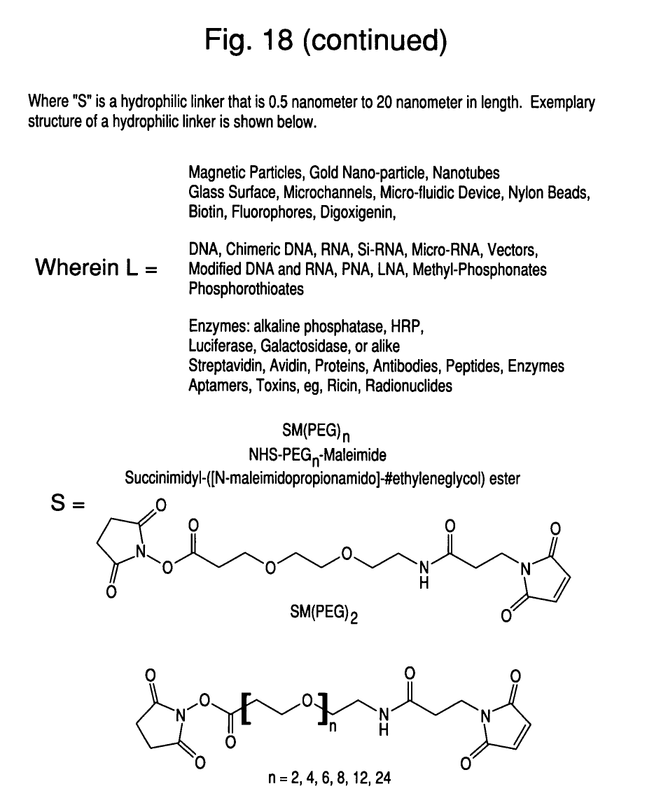

After immunization, 3-5 ml shark blood will be withdrawn from the animal and the total IgGs will be precipitated out using 50% ammonium sulfate, followed by centrifugation at 2000 RPM for 10 minutes. After discarding supernatant, the precipitate will be dissolved in 20 mM PBS/150 mM NaCl containing 0.02% sodium azide and size fractionated on Sephadex G-200. The conventional IgGs, MW ˜230 KDa, will be separated out from the shark IgNAR with MW of ˜180 K Da. Alternatively, the conventional IgG fraction will first be depleted with protein G bound to magnetic beads, followed by isolation of V-NAR protein with magnetic beads coated with protein-A. Affinity purification to obtain high affinity shark Ig-NAR 2 will be done by magnetic beads coated with antigenic peptide. After determining the amino acid sequence of the IgNAR, nucleic acid sequence will be derived based on amino acid sequence and recombinant DNA protocols will be established to produce the antibody on a large scale. A schematics for the method of developing several analogs are shown in FIGS. 17 and 18.

### Example 14

### Isolation of RNA from Immunized Shark's Lymphocytes and Cloning

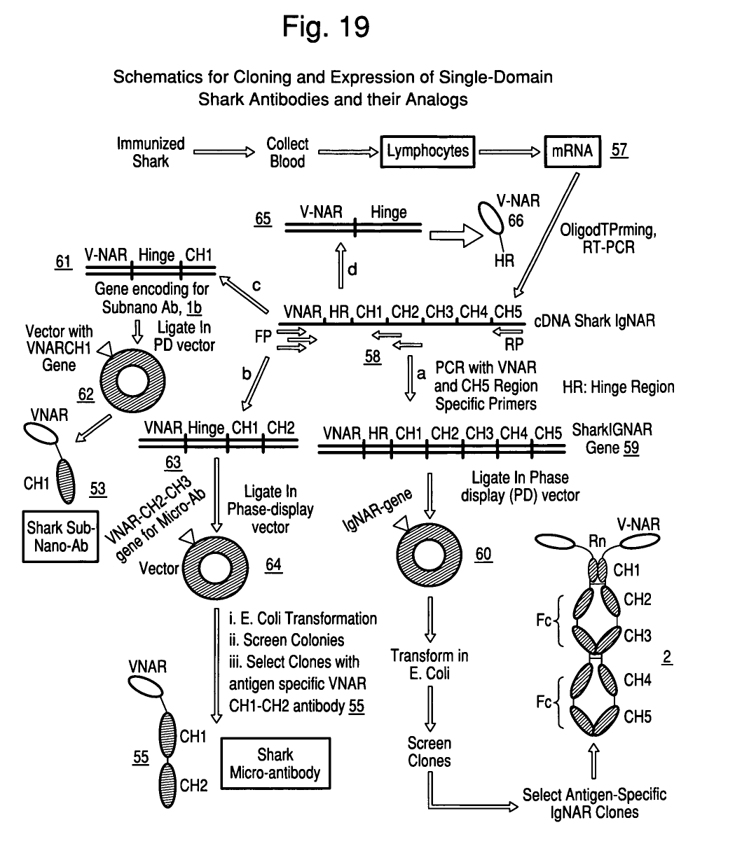

Isolation of total RNA from immunized sharks will be done from 3-5 ml of shark blood using commercially available RNA extraction kits such as Bio-Rad's AquaPure® RNA Isolation kit. Reverse transcription using oligo-dT primer will be achieved by PCR using high fidelity DNA polymerase to obtain the IgNAR cDNA 58 shown in FIG. 19.

### Example 15

### Recombinant Production of Shark Heavy Chain Only Antibodies and their Analogs

An exemplary cloning strategy is shown in FIG. 19. Amplicons for IgNAR cDNA 58 and its analogs will be performed using the following protocol:

IgNAR cDNA=1.0 ug

Primers Mix=10 pmol (forward and reverse primers)

1 mM dTNPs=10 ul

10 mM MgCl2=5 ul

10×PCR Buffer=5 ul

Taq DNA Polymerase=0.6 ul

Water to=50 ul

After first denaturation round of 94° C. for 10 minutes, 35 to 36 cycles of amplification will be performed under conditions as described below:

| | |
|---|---|
| Denaturation: | 20 seconds at 94° C. |
| Annealing: | 30 Seconds at 56° C. |
| Extension: | 50 seconds at 72° C. |
| Final Extension: | 10 min, 72° C. |

All or portions of IgNAR cDNA using different combinations of the following forward and reverse primers.

| | |
|---|---|
| Forward Primers | |
| 5′-gcatgggtag accaaacaccaag-3′ | (SEQ ID NO: 18) |
| 5′-gcgtcctcagagagagtcccta-3′ | (SEQ ID NO: 19) |
| 5′-gagacggacgaatcactgaccatc-3′ | (SEQ ID NO: 20) |
| 5′-gggtagaccaaacaccaagaacagc-3′ | (SEQ ID NO: 21) |
| Reverse Primers | |
| 5′-gttctagccaataggaacgtatag-3′ | (SEQ ID NO: 22) |
| 5′-gtttgcacaagagagtagtctttac-3′ | (SEQ ID NO: 23) |
| 5′-cctaaattgtcacagcgaatcatg-3′ | (SEQ ID NO: 24) |
| 5′-gtgcagttccctagaagtcttg-3′ | (SEQ ID NO: 25) |

After amplification, the amplicon will be purified on 1.5% agarose. The amplicon will be extracted from the gel and its 5′-end kinased with gamma-ATP for blunt-end ligation with the phage-display vector using T4 DNA-ligase following standard ligation protocols.

Library or Plasmid Construction: Prior to cloning, the PCR amplicon encoding IgNAR gene will be digested with Sfi1 and Not1 (Roche) following the cocktail:

| | V-NAR-CH2—CH3— | | Vector | |
|---|---|---|---|---|
| DNA | 5 | ug | 10 | ug |
| 10× Restriction Buffer | 5 | ul | 5 | ul |
| Sfi1 (10U/ul) | 8 | ul | 4 | ul |
| Water to | 50 | ul | 50 | ul |
| Incubate 50° C. for 8 hour | | | | |
| Not1 | 35 | U | 30 | U |
| Reaction Buffer | 4.5 | ul | 4.5 | ul |
| Water to | 60 | ul | 60 | ul |
| Incubate at 37° C. for 4-5 | | | | |
| hours. | | | | |
| Ethanol Precipitate at −70° C. | Pellet | | Pellet | |
| Water | 50 | ul | 50 | ul |

| | | |
|---|---|---|
| Agarose gel (1.5%) | Pure DNA | Pure Vector DNA |
| purification | Encoding micro-HCAb | |

Vector Ligation:

| | | |
|---|---|---|
| IgNAR DNA = | 200 | ng |
| Vector DNA = | 1000 | ng |
| 10× Ligase Buffer = | 5 | ul |
| T4 DNA Ligase = | 10 | U |
| Water to 50 ul | | |
| Incubate 15 hours at 4° C.. | | |
| Ethanol Precipitate at −70° C. | | |
| Suspend pellet in 10 ul. | | |

### Electroporation:

250 ul of E. Coli TG1 cells will be made electrocompetent with BRL Cell-Porator® following vendor protocol.

Panning of Phage-Displayed IgNAR-Antibody 2 Library: Electroporated TG1 cells will be transfected with the phagemid-IgNAR DNA insert. Approximately, 10¹⁰ cells will be grown to mid-logarithmic phase before injection with M13K07 helper phages. Virions will be prepared as described in the literature [Andris-Widhopf J., et al, J. Immunology Methods, 242, 159 (2002)] and used for panning at a titer of 10¹³/ml. Specific IgNAR antibody against the antigenic peptide will be enriched by five consecutive rounds of panning using magnetic beads conjugated with antigenic peptide. Bound phage particles will be eluted with 100 mM TEA (pH 10.00), and immediately neutralized with 1M Tris.HCl (pH 7.2) and will be used to reinfect exponentially growing E. Coli TG1 cells.

The enrichment of phage particles carrying antigen-specific IgNAR antibody will be assessed by ELISA before and after five rounds of panning. After the fifth panning, individual colonies will be picked up to analyze the presence of the virion binding by anti-M13-HRP conjugate.

### Example 16

### Expression and Purification of the Single-Domain IgNAR 2

The selected positive clones will be used to infect a new bacterial strain, HB 2151, a non-suppressor strain that recognizes the amber codon as a stop codon for soluble protein production. The HB2151 cell harboring the recombinant phagemids will be grown at 28° C. in 250 ml 2×YT-ampicillin, 1% glucose in culture flasks until OD₆₀₀ 0.7. The cells will be washed and resuspended in 250 ml 2×YT-ampicillin, supplemented with 1 mM isopropyl-BD-thiogalactopyranoside (IPTG), and incubated over night at 22° C. to induce protein expression. Before adding IPTG to the cultures, a portion will be spotted on an LB/ampicillin plate for future analysis of the clones. The culture will be then be centrifuged at 4000 RPM for 15 minutes to pellet the bacterial cells. The culture supernatant will then be screened by ELISA for antigen-specific IgNAR protein 2.

### Example 17

### Chemical Synthesis of Shark Heavy Chain Only Antibodies and Their Analogs

### TCEP Treatment:

Shark IgNAR Mini-antibody 52 will be prepared by treating parent antibody 2 (2 mg) with 1.0 ml of 10 mM TCEP (tris-carboxyethyl-phosphine) in 20 mM phosphate/150 mM NaCl, pH7.4 at room temperature (RT) for one hour as shown in FIG. 18. The resulting mini-antibody 52 will be desalted on centricon-10 filters to remove the excess reagent and the buffer and stored at 4° C. in 1×PBS containing 0.05% NaN₃.

### Chemical Synthesis of Micro-Antibody, 55:

Mini-antibody 52 will be treated with pepsin under controlled conditions to cleave the CH3-CH4-CH5 domain from the antibody. After deactivation of the proteolytic enzyme with fetal calf serum, the micro-antibody 55 will be isolated using size exclusion chromatography and stored at 4° C. in PBS containing 0.05% NaN3.

### Chemical Production of Shark Sub-Nano-Antibody, 53:

Micro-antibody 55 will be treated with trypsin under milder conditions at pH 4.5 to cleave the CH2 domain from the antibody 55. After deactivation of the proteolytic enzyme with fetal calf serum, the sub-nano-antibody 53 (V-NAR-HR-CH1) will be isolated using size exclusion chromatography and stored at 4° C. in PBS containing 0.05% sodium azide.

### Chemical Production of Shark Nano-Antibody, 72:

Mini-antibody 53 will be treated with tropism/papain/pepsin under milder conditions at pH 4.5 to cleave the CH1 domain from the antibody 53. After deactivation of the proteolytic enzyme with fetal calf serum, the nano-antibody 72 (V-NAR-HR-CH1) will be isolated using size exclusion chromatography and stored at 4° C. in PBS containing 0.05% sodium azide.

### Attachment of Hydrophilic Linker to Shark Antibodies 2, 52, 53, 54, 55, and 72:

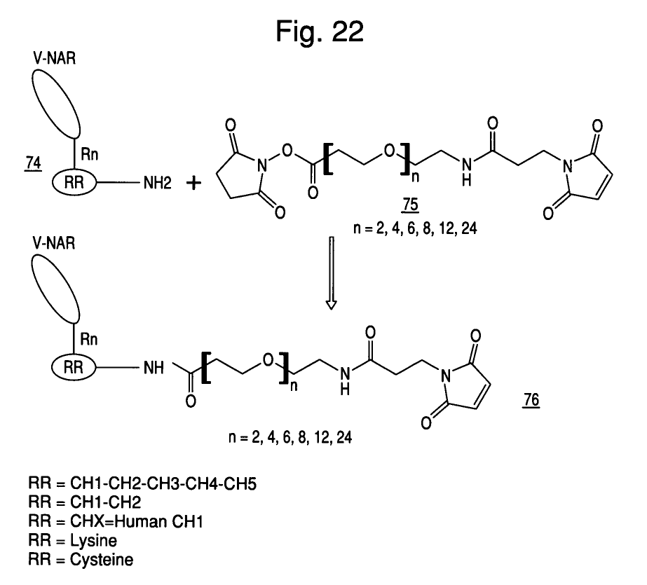

Shark antibody (5 nM), produced using cloning techniques and/or chemical methods, will be dissolved in 0.5 ml of 50 mM MOPS/150 mM NaCl, pH 7.0, and treated with NHS-(PEG)n-Maleimide (50 nM, 10×) at RT for 1 hour with gentle shaking of the reaction contents. The reaction mixture will then be dialyzed/concentrated on centricon-3 to remove excess of the reagent to obtain antibody analogs 67, 68, 69, 70, and 72 shown by their general structures in FIG. 18. The conjugation schematics are shown in FIG. 22.

### Chemical Synthesis of Bivalent V-NAR 78

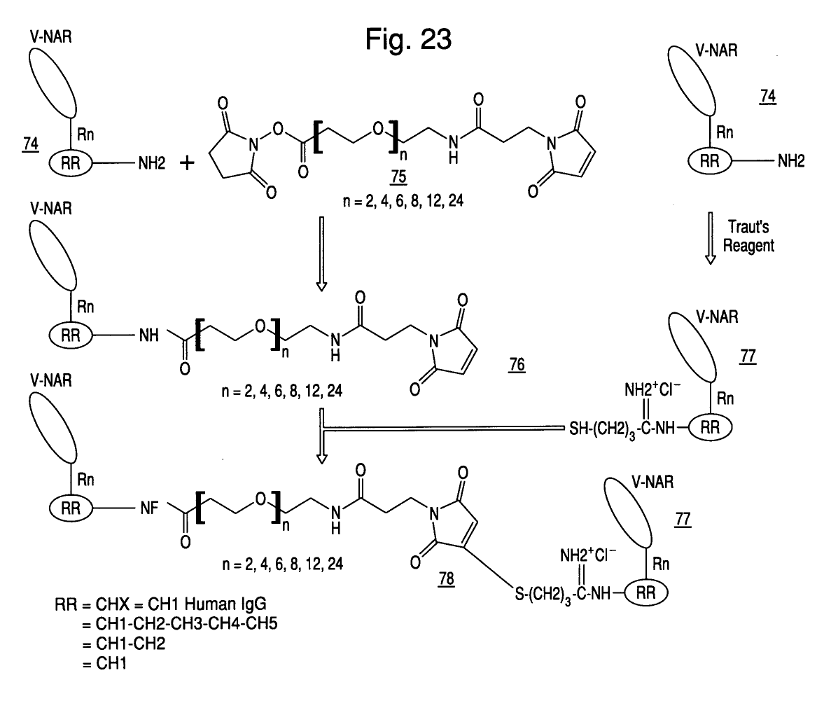

V-NAR 74 will be treated with NHS-PEG-Mal in 50 mM MOPS/150 mM NaCl, pH 6.8, at RT for 1 hour to obtain the pegylated conjugate 76 (FIG. 17) which will be desalted by dialysis on C-3 Amicon filters. Also, nano-antibody 74 will be modified with Traust's Reagent following manufacturer protocol to obtain thiolated nano-antibody 77. Conjugation of 76 with 177 in pH 6.5 buffer for 2 hours at RT will give, after dialysis on C-3 Centricon membranes, the dimeric conjugate 78 which will then be tested by ELISA and Western blot assays. The reaction schematics are shown in FIG. 23.

### Chemical Synthesis of Multivalent V-NARs:

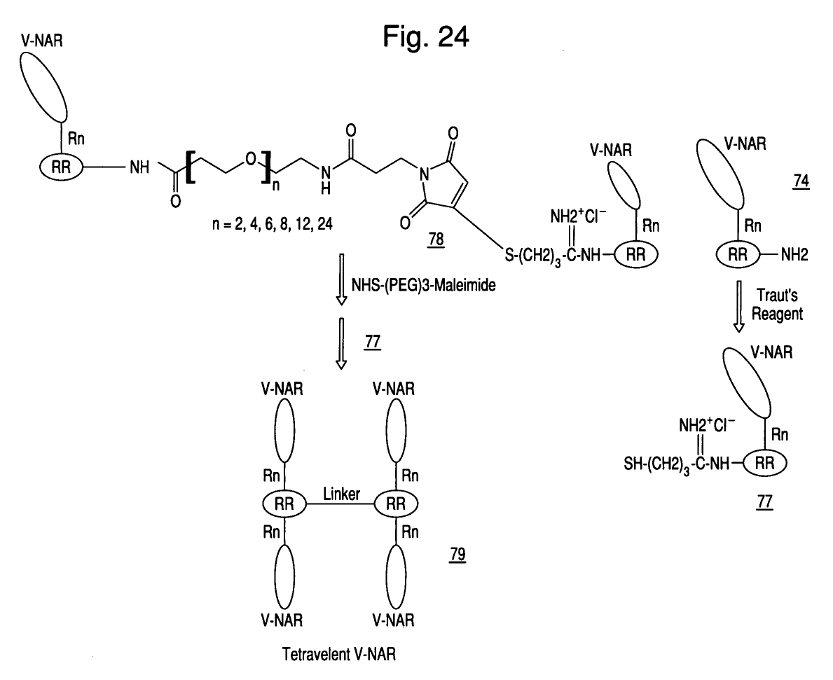

Tetravalent and pentavalent shark antibodies can be generated from the bivalent analog, 78. Bivalent V-NAR 78 will be treated with short NHS-PEG_{S}-Mal as described above. After the reaction is over, the pegylated dimer will be dialyzed on centricon-10, followed by treatment with the thiolated V-NAR nano-antibody 77. The tetravalent conjugate 79 so obtained will be purified by size exclusion chromatography. The reaction schematics are shown in FIG. 24.

### Example 18

### Capture and Detection of Pathogenic Antigens/Proteins Using Shark and Camel Single-Domain Antibodies (sdAbs)

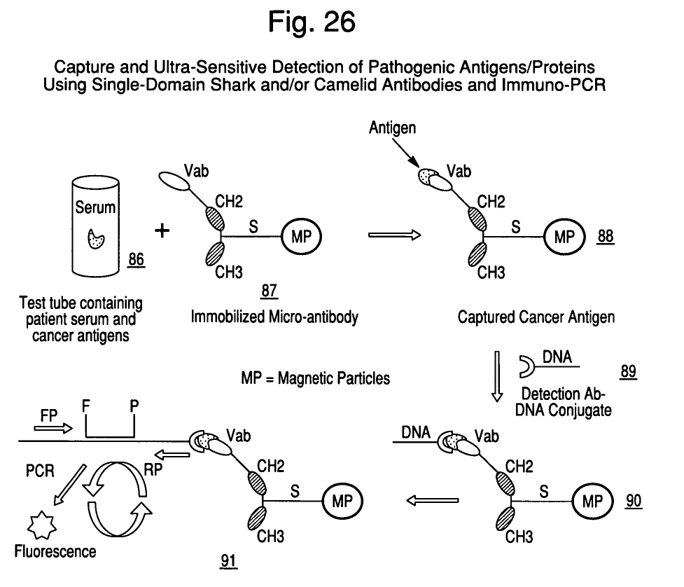

Serum from patient blood (10 ml), collected in EDTA tubes will be treated with shark and camelid heavy chain only antibodies and their analogs coated magnetic beads for 1-2 hours on a rotator with gentle rotation to bind the antigen. The beads will be separated using a magnetic rack and subsequently washed very well with PBS/1% BSA. The antigen-micro-antibody complex so formed will be treated with complex, detection antibody bound to an enzyme (AP, HRP, Luciferase, beta-galactosidase, gold particles) or DNA to sandwich the antigen between the shark and camelid heavy chain only antibodies and their analogs and the detection antibody forming the complex which will be detected either using an enzyme substrate or AgNO3 if the detection antibody is conjugated to gold particles. Exemplary schematics of the process is shown in FIG. 25. Alternatively, the detection antibody could be conjugated to DNA molecules which can then be amplified by PCR to obtain detection sensitivity equivalent to the detection of DNA by PCR as shown in FIG. 26.

### Example 19

### Capture and Detection of Rare Cells Using Shark and Camelid Heavy Chain Only Antibodies and their Analogs

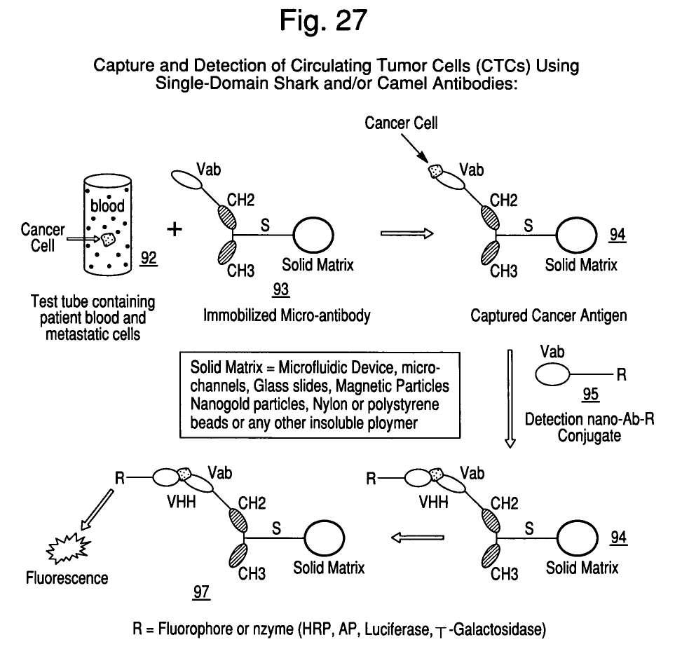

Fresh 5 ml patient blood will be diluted with 20 ml 1×PBS/1% BSA to 25 ml. To capture circulating tumor cells (CTCs), this sample will then be passed through a micro-fluidic device coated with an appropriate shark and camelid heavy chain only antibodies and their analogs, such as, anti-EpCAM-mini-antibody following flow rate recommended by the manufacturer. To ensure that antibody or its analogs does not lose any activity upon conjugation, all solid matrixes will first be coated with a hydrophilic polymer, such as, NHS-PEG-Mal (MW 5000). The conjugation of the thiolated shark and camelid heavy chain only antibodies and their analogs with maleimido-group of the polymer will be achieved at pH 6.8 in a buffer containing 5% EDTA. An exemplary schematics of the process is shown in FIG. 27.

Alternatively, magnetic beads coated with EpCAM can be used. EpCAM (epithelial cell adhesion molecules) is frequently over expressed by carcinomas of lung, colorectal, breast, prostate, head and neck, liver, and is absent from hematological cells. The captured cells will be washed with 1% PBS (no BSA). The cell will be fixed with methanol, and then DAPI stained following CK8 or CK18 and CD45. Identification and enumeration will be done by fluorescence microscopy based upon the morphological characteristics, cell size, shape, and nuclear size. DAPI⁺, CK⁺, and CD45⁻ cells will be classified as CTCs.

Method for capturing fetal cells will be the same except the antibody used for coating micro-fluidic device will be different, for example, anti-CD71-mini-antibody, an antibody that recognize transferrin receptor on fetal cells. The captured cells can be enumerated by FISH or PCR.

Alternative Strategies to Capture Circulating Tumor Cells (CTCs): CTCs will be captured as shown by the steps outlined in platform 3. Patient's blood (2-3 ml) (or urine 15-20 ml after centrifugation to pellet down the cells and suspending them in 1-2 ml HBSS media) will be incubated with an appropriate mini-HCAb (1.5 ug/ml blood sample) at 4° C. for one hour. For example, to capture epithelial cancer cells, such as from breast, prostate, and ovarian cancers, biotinylated-anti-EpCAM-mini-antibody (camel antibody against EpCAM antigens) will be used to label the circulating cancer cells in the blood. After diluting with HBSS or RPMI-1640 media or 1×PBS/2.5% BSA to lower the sample viscosity, the diluted blood is then passed through a microfluidic device coated with streptavidin at a flow rate allowing maximum cell capture. The captured CTCs can then be fixed by fixing with methanol, followed by fixing with 1% PFA using any standard cell fixing procedures. Enumeration will then be done by DAPI staining followed by immunohistochemical staining with commonly used mouse mHCAb such as CK-7 but more preferably mini-CK-7 for higher specificity. CTCs have to be CD45 negative.

Alternatively, most of the RBCs from the blood sample can be first lysed using ammonium chloride solution (155 mM NH₄Cl/10 mM NaHCO₃). After pelleting, the washed cells will be suspended in HBSS media (1-2 ml) and passed through the microfluidic device coated with heavy-chain antibody specific for the cell type one needs to capture and analyze.

Alternatively, the diluted blood sample after incubation with the biotinylated-anti-EpCAM-mini-antibody or micro-antibody will be treated with the streptavidin coated magnetic particles (Miltenyl) for 30 minutes at 4° C. while the sample is being gently rotated on a rotating wheel. After pulling down the magnetic particles with a magnet, the CTCs bound to the particles will be washed with PBS/1% BSA. The CTCs can then be enumerated by spreading them in a unilayer on a glass slide, drying them for one to two hours, followed by fixing with methanol, 1% PFA and staining the CTCs with CK-7.

Furthermore, these captured CTCs can be analyzed for the gene expression. For example, in case of prostate cancer patient, one can look for TMPRSS2-ERG translocation using PCR primers. TMPRSS2-ERG transcript is present in about 50% of the prostate cancer patients. Similarly, one can look for HER-2 expression in case of breast cancer.

### Capture of Fetal Cells:

Blood samples (10-15 ml) from pregnant mothers (7 to 12 weeks gestation) will be collected in Cytochex blood collection tubes and transported for overnight delivery at 4° C. Upon arrival, samples will be incubated with a mixture of biotinylated-mini- or micro-anti-CD34, CD133, Trop-1, CD71 and 6B5 antibodies (1 ug each/ml blood) at 4° C. for one hour during which time the sample will be gently rotated. After diluting with HBSS or RPMI-1640, the blood sample will be passed through a microfluidic device (microchannels) coated with hydrophilic polymer (PEG, MW 500 to 5000, or a short oligonucleotide fragment 4 to 20 bases long or a short peptide, etc) and streptavidin. The captured cells will be fixed with methanol followed by fixation with 1% PFA. After staining with epsilon- and gamma-hemoglobins, the cells will be subjected to fluorescence in-situ hybridization with Vysis FISH probes for chromosomes X, Y, 13, 18 and 21. Fetal male gender will be readily detected by the appearance of X and Y fluorescence signals under the fluorescence microscope. XX cells stained with epsilon-hemoglobin will be classified as female fetal cells. Trisomy signals will be evident from three identical FISH signals under the microscope.

Alternatively, most of the RBCs can either be carefully lysed using a mild treatment with ammonium chloride lysis reagent (155 mM NH₄C1/10 mM NaHCO₃) to enrich for fetal nucleated red blood cells (fnRBCs) before incubating the sample with a mixture of biotinylated antibodies.

Still another option will be the use of a density gradient such as Ficol 1.073 or Percol 1.073. The buffy coat can then be processed as above to yield fetal nRBCs.

### Example 20

### Detection of Chromosomal Translocations from Captured Circulating Tumor Cells (CTCs) Using Shark and Camelid Heavy Chain Only Antibodies and their Analogs

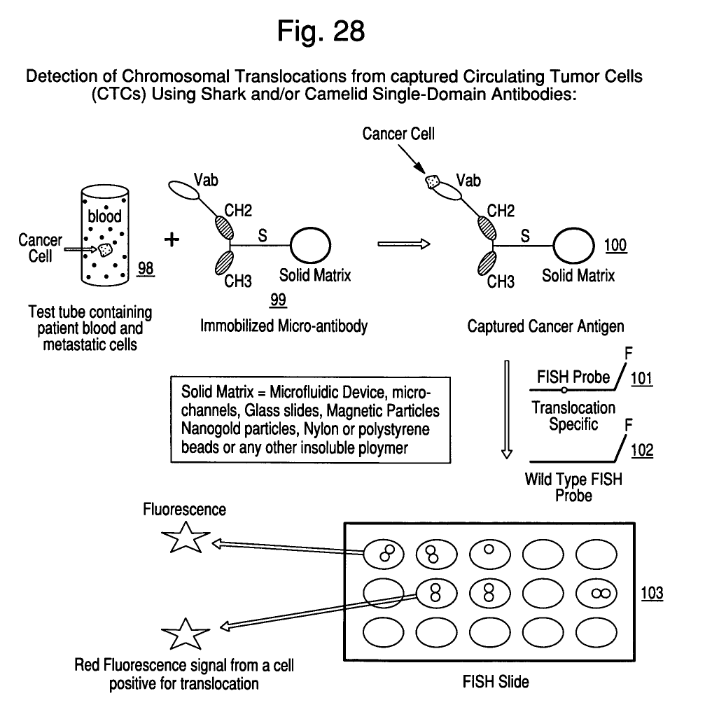

Cells will be captured as described above and also shown in FIG. 27-28. Enumeration will be done using an appropriate FISH probes. For example, while investigating TMPRESS2-ERG translocation in case of prostate cancer, FISH probes designed to hybridize with the junction region will be used. Similarly, in case of CML, bcr-Abl FISH probe will be used. An exemplary schematics of the process is shown in FIG. 28.

### Example 21

### Detection of Non-Invasive Prenatal Genetic Disorders from Captured Circulating Fetal Cells (CFCs) Using Heavy-Chain Antibodies

Blood (10 ml) from a pregnant woman will be treated at RT for 1 hour with sdAb conjugated to magnetic beads, with gentle shaking. The beads will be allowed to settle down in a magnetic rack and then subsequently washed with a wash buffer containing 20 mM PO4⁻²/150 mM NaCl/0.1% Triton X-100 (3×2 ml) to ensure complete removal of blood and serum. The beads will then be washed with 1×PBS to remove triton. The bound DNA will then be eluted by hot 10 mM Tris.HCl, pH7.0 or by protease digestion.

This fetal DNA will then be analyzed by real-time PCR using Y-chromosome primers to test the gender and by chromosome 21 primers to test for Down syndrome.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

1-60. (canceled)

61. A polypeptide comprising all or a portion of at least one variable antigen-binding (Vab) domain of camelid and/or shark single-domain heavy chain antibodies lacking light-chains, at least ten contiguous amino acids derived from a source other than camelid and/or shark single-domain heavy chain antibodies lacking light-chains, wherein said polypeptide comprises at least one binding site for an antigen.

62. The polypeptide of claim 61, wherein said polypeptide is linked to at least one entity other than an antibody.

63. The polypeptide of claim 62, wherein said entity is selected from a group consisting of solid support, radioisotope, an enzyme, detectable label, ligand, fluorophore, biotin, digoxegenin, avidin, streptavidin, Fc region of IgGs, a therapeutic agent, toxin, hormone, peptide, protein, antibody, vector, siRNA, micro-RNA and nucleic acid.

64. A polypeptide comprising all or a portion of at least two variable antigen-binding (Vab) domains of camelid and/or shark single-domain heavy chain antibodies lacking light-chains, at least ten contiguous amino acids derived from a source other than camelid and/or shark single-domain heavy chain antibodies lacking light-chains, all or a portion of at least one hinge region of camelid and/or shark single-domain heavy chain antibodies lacking light-chains in a single polypeptide chain, wherein said at least two Vab domains bind to at least two different antigens, and wherein said polypeptide has improved biodistribution and retention.

65. The polypeptide of claim 64 comprising all or a portion of at least three and more variable antigen-binding (Vab) domains of camelid and/or shark single-domain heavy chain antibody, wherein said polypeptide has improved biodistribution and retention.

66. A composition comprising at least two polypeptides, wherein each of said polypeptides comprises variable domain (Vab) of camelid and/or shark single-domain heavy chain only antibody, all or a portion of at least one hinge region of camelid and/or shark heavy chain only antibody, wherein at least one of said polypeptide comprises at least one binding site for an antigen, wherein said composition has improved biodistribution and retention, and wherein said polypeptides are linked to each other through at least one linker.

67. A nucleic acid encoding all or portion of said polypeptide of claim 61.

68. A method for producing a polypeptide of claim 61, said method comprising transforming a host cell with a recombinant nucleic acid encoding said polypeptide, and expressing said polypeptide in said host cell.

69. A method for producing a polypeptide of claim 64, said method comprising transforming a host cell with a recombinant nucleic acid encoding said polypeptide, and expressing said polypeptide in said host cell.

70. A method for diagnosing an individual with a disease, said method comprising:
a) obtaining a sample from said individual
b) detecting the presence or absence of a biomarker for said disease, wherein said detecting comprises utilizing a polypeptide claim 61, wherein said polypeptide binds specifically to said biomarker;
c) determining the level of said biomarker if present in said individual's sample;
d) comparing said level to a reference value; and
e) identifying said individual as having said disease when the level of said biomarker in said individual's sample is higher than said reference value.

71. A method for detecting the presence or absence of circulating tumor cells in a sample comprising
a) obtaining a sample suspected of having circulating tumor cell,
b) detecting the level of one or more tumor cell surface receptors in said sample utilizing a polypeptide claim 61, wherein at least one of said Vab domain binds specifically to tumor cell surface receptor, wherein the said receptor is selected from a group consisting of MUC-1, VCAM-1, EpCAm-1, CD44, CD133, E-Cadherin, VEGF, bFGF, sFASL, CD95, p53, Bcl-2 CyclinD1, Cyclin E, TNF-alfa, TGF-beta1, Her-2, EGFR, IGF-1 and IGF-1R, IL-2R, Ras, and cMyc, wherein the level of said tumor cell surface receptor in said sample is indicative of the presence or absence of circulating tumor cells.

72. A method for detecting the presence or absence of circulating rare fetal cells in a sample comprising
a) obtaining a sample suspected of having circulating rare fetal cells,
b) detecting the level of one or more tumor cell surface receptors in said sample utilizing a polypeptide claim 61, wherein at least one of said Vab domain binds specifically to fetal cells cell surface receptors, wherein the said receptors is selected from a group consisting of GPA, CD71, CD133, CD34, CD44, ITCAM, ITGB1 (Integrin beta-1), Trop-1, Trop-2, HLA-G233, and 6B5, wherein the level of said fetal cell surface receptor in said sample is indicative of the presence or absence of circulating fetal cells.

73. A method for detecting the presence or absence of an antigen associated with a disease in a sample comprising:
a) obtaining a sample suspected of having said antigen,
b) detecting the level of said antigen in said sample utilizing a composition of claim 61, wherein said polypeptide binds specifically to said antigen, wherein the level of said antigen in said sample is indicative of the presence or absence of said antigen.

74. A method for diagnosing an individual with a disease, said method comprising:
a) obtaining a sample from said individual;
b) detecting the presence or absence of a biomarker for said disease, wherein said detecting comprises utilizing a composition of claim 64, wherein at least one of said polypeptides binds specifically to said biomarker;
c) determining the level of said biomarker if present in said individual's sample;
d) comparing said level to a reference value; and
e) identifying said individual as having said disease when the level of said biomarker in said individual's sample is higher than said reference value.

75. The polypeptide of claim 64, wherein said polypeptide is linked to an entity other than an antibody.

76. The polypeptide of claim 75, wherein said entity is selected from a group consisting of solid support, radioisotope, an enzyme, detectable label, a therapeutic agent, toxin, hormone, and nucleic acid.

77. The composition of claim 66, wherein at least one of said polypeptide is linked to an entity other than an antibody.

78. A method of simultaneously diagnosing, preventing, treating, and/or alleviating symptoms associated with a disease in an individual, said method comprising:
a) administering to said individual in need thereof said polypeptide of claim 61,
b) detecting the presence or absence of a biomarker for said disease, wherein said detecting comprises utilizing a polypeptide claim 61, wherein said polypeptide binds specifically to a biomarker associated with said disease;
c) determining the level of said biomarker if present in said individual's sample;
d) comparing said level to a reference value;
e) identifying said individual as having said disease when the level of said biomarker in said individual's sample is higher than said reference value; and
f) preventing, treating, and/or alleviating symptoms associated with said disease in said individual when said polypeptide specifically binds to said biomarker.

79. A method of simultaneously diagnosing, preventing, treating, and/or alleviating symptoms associated with a disease in an individual, said method comprising:
a) administering to said individual in need thereof said polypeptide of claim 64,
b) detecting the presence or absence of a biomarker for said disease, wherein said detecting comprises utilizing a polypeptide claim 64, wherein said polypeptide binds specifically to a biomarker associated with said disease;
c) determining the level of said biomarker if present in said individual's sample;
d) comparing said level to a reference value;
e) identifying said individual as having said disease when the level of said biomarker in said individual's sample is higher than said reference value; and
f) preventing, treating, and/or alleviating symptoms associated with said disease in said individual when said polypeptide specifically binds to said biomarker.

80. A method of simultaneously diagnosing, preventing, treating, and/or alleviating symptoms associated with a disease in an individual, said method comprising:
a) administering to said individual in need thereof said composition of claim 66,
b) detecting the presence or absence of a biomarker for said disease, wherein said detecting comprises utilizing said composition of claim 66, wherein said polypeptide binds specifically to a biomarker associated with said disease;
c) determining the level of said biomarker if present in said individual's sample;
d) comparing said level to a reference value;
e) identifying said individual as having said disease when the level of said biomarker in said individual's sample is higher than said reference value; and
f) preventing, treating, and/or alleviating symptoms associated with said disease in said individual when said polypeptide specifically binds to said biomarker.


SEQUENCE LISTING (106 sequences)

SEQ ID NO: 1 - protein, 129 aa, Camelus dromedarius
DVQLQESGGGSVQAGGSLKLSCAISGYDNDNYCMGWFRQTPGKEREKVAALNIGGGSPVYADFVRGRFTISLDSSKDTLYLLMNAVTPEDTAMYYCAAIRKPQFYTCRMWKPRADFDIWGQGTQVTVSS

SEQ ID NO: 2 - protein, 35 aa, Camelus dromedarius
EPKIPQPQPKPQPQPQPQPKPQPKPEPECTCPKCP

SEQ ID NO: 3 - DNA, 387 bp, Camelus dromedarius
gatgtgcagctgcaggagtctggaggaggctcggtgcaggctggaggctctctgaaactctcctgtgcaatttctggatacgacaacgataactactgcatgggctggttccgccaaacgccagggaaggagcgtgagaaagtcgcggcccttaatattggaggtggtagcccagtctacgccgatttcgtgaggggccgattcaccatctccctggactccagcaaggacacactgtacctcctgatgaacgcagtgacacccgaggacacggccatgtattactgtgcggcaatccgtaagccccaattctatacttgccgtatgtggaaaccaagagctgactttgatatctggggccaggggacccaggtcaccgtctccagc

SEQ ID NO: 4 - DNA, 104 bp, Camelus dromedarius
ggacagaagacaccgcaccaacggccaagaccccacccccaacagcgaccgcagccgagacagcggcagagacacgaaccggagtgcacgtgtcccagatgtcc

SEQ ID NO: 5 - DNA, 19 bp, Artificial Sequence
  Description of Artificial Sequence Synthetic primer
gcaggagtctggaggaggc

SEQ ID NO: 6 - DNA, 21 bp, Artificial Sequence
  Description of Artificial Sequence Synthetic primer
ggacatctgggacacgtgcac

SEQ ID NO: 7 - DNA, 294 bp, Homo sapiens
gcctccaccaagggcccatcggtcttccccctggcaccctcctccaagagcacctctgggggcacagcggccctgggctgcctggtcagggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtt

SEQ ID NO: 8 - DNA, 27 bp, Artificial Sequence
  Description of Artificial Sequence Synthetic oligonucleotide
ctcgaggcctccaccaagggcctcgag

SEQ ID NO: 9 - DNA, 30 bp, Artificial Sequence
  Description of Artificial Sequence Synthetic oligonucleotide
gcctccaccaagggcccatcggtcttcccc

SEQ ID NO: 10 - DNA, 42 bp, Artificial Sequence
  Description of Artificial Sequence Synthetic oligonucleotide
gcctccaccaagggcccatcggtcttccccctggcaccctcc

SEQ ID NO: 11 - DNA, 60 bp, Artificial Sequence
  Description of Artificial Sequence Synthetic oligonucleotide
gcctccaccaagggcccatcggtcttccccctggcaccctcctccaagagcacctctggg

SEQ ID NO: 12 - DNA, 60 bp, Artificial Sequence
  Description of Artificial Sequence Synthetic oligonucleotide
ggcacagcggccctgggctgcctggtcagggactacttccccgaaccggtgacggtgtcg

SEQ ID NO: 13 - DNA, 60 bp, Artificial Sequence
  Description of Artificial Sequence Synthetic oligonucleotide
tggaactcaggcgccctgaccagcggcgtgcacaccttcccggctgtcctacagtcctca

SEQ ID NO: 14 - DNA, 60 bp, Artificial Sequence
  Description of Artificial Sequence Synthetic oligonucleotide
ggactctactccctcagcagcgtggtgaccgtgccctccagcagcttgggcacccagacc

SEQ ID NO: 15 - DNA, 54 bp, Artificial Sequence
  Description of Artificial Sequence Synthetic oligonucleotide
tacatctgcaacgtgaatcacaagcccagcaacaccaaggtggacaagaaagtt

SEQ ID NO: 16 - protein, 661 aa, Orectolobus maculatus
AWVDQTPRTATKETDESLTINCVLRESPYELYNTGWYRTKLGSTKEQRISIGGRYVETVDKESKSFSLRISDLRIEDSGTYKCGACDEPDGGYGKYSCFTYKKGTGTGLTVKPGVQPSPPVISLLYSATEEQRGNGFVQLICLISGYYPENIAVSWQKNRNDISSGFTTSPSMKTSTNDFSSTSLLNVPLQEWSSGSVYSCRVSHSATNSNQRKEIRSTSEIAVFLRDPSVEEIWINKSATVVCEVLSTVSTGVVISWMVDGKVRTEGVRIEAAKMDGNQYLTISRLSSSVEEWQSGVEYTCSAKQDQSSTPVSKRTGKTKVEPMKPYLRLLPPSPEEIQNISSAILTCLIRGFYPDKIRVSWEKDGAAVSGNITSFPTALEQDLTFSTRSLLILPAVEWKSGAKYTCIASHPPSQSTVKRVIRSPKGDCGQPDISVNLLNPPFEEIWTQKTATIVCEIVYSDLENVNVFWQVNGSERTEGVETQNPEWSGSKSTIVSKLKVTSSEWDSGVEYVCLVEDSELPTPVKSSIRKAKDREMYPPKVYVLHPSTDEIDTENSATLVCLATGFSPAEIYVGWMANDTLLNSGYRSQVENEKGNGSNFIINRLRLTAAEWDSDTTYSCLVGHPSLSRDLIRSINKSHGKPTLVNLSVVLSDTVKSCT

SEQ ID NO: 17 - DNA, 2328 bp, Orectolobus maculatus
gcatgggtagaccaaacaccaagaacagcaacaaaagagacggacgaatcactgaccatcaattgcgtcctcagagagagtccctacgaattgtacaacacgggctggtatcggacaaaattgggttcaacaaaggagcagagaatatcaattggcggacgatatgttgaaacagtcgacaaagaatcaaagtccttttctctgagaattagtgatctgagaattgaagacagtggcacgtataagtgtggagcatgtgatgaacctgacgggggctacggtaagtatagctgtttcacctacaagaaaggaactggcaccggactgaccgtgaaacctggagtacagccttctccaccagtcatcagtctactttactctgcaactgaagaacagagaggaaacgggtttgtgcagctgatttgtctaatcagcggatactatcctgaaaacattgcagtgagctggcaaaagaacagaaacgacataagttctggctttacaacttcaccctcaatgaaaacatcgaccaatgactttagctctacaagtttacttaatgtgcccctgcaggaatggagcagcggttctgtgtacagttgtcgagtttctcattctgcaaccaacagtaaccaaaggaaagaaattagatcaacatcagagattgctgtattcctaagagatccatcagttgaagaaatctggatcaataaaagtgccactgtggtttgcgaagttctttctacagtttccactggagtcgtcatctcttggatggtggatggaaaggtaaggaccgaaggcgttcgaatcgaagcagctaaaatggatggaaaccaatatctgaccatcagccgcttgagcagcagcgtggaagagtggcagagtggggtggaatacacatgctccgcaaaacaggatcaatcatcgaccccggtatcaaaacgaacaggaaaaacaaaagtcgagccaatgaagccatatcttcgcctcctgcccccatcaccagaggagattcaaaacatcagttctgctattctcacatgtttgataagaggattctaccctgacaagatacgcgtttcctgggagaaggacggagctgctgtgagtgggaacatcaccagtttcccgactgctctggaacaggacctgaccttcagcacccggagcctcctcattttgcctgcagtggaatggaagagcggagcaaaatacacctgtatcgcctcacatccaccgtcacaatccactgtgaagagggtcatcaggagcccgaaaggtgattgcggtcagccagacatttctgtcaatctactgaaccctccgtttgaagagatttggacacaaaagacagcgaccattgtttgtgaaatcgtttacagtgacttagaaaacgtcaacgtgttctggcaggtgaatgggagtgagagaacggagggagtcgagacacaaaatcctgagtggagtggaagcaaatccaccattgtcagcaaactaaaagtaacgtcttcggagtgggacagtggtgtggaatatgtctgcttggtagaagacagtgaattaccaacaccagtgaaatcgtccatcaggaaggcaaaggaccgcgaaatgtaccctcctaaggtttatgtcctgcatccatcgacggacgagattgacactgagaattcggctaccctggtgtgtctagccaccggcttttccccagctgagatttacgtcggttggatggccaatgacacacttttgaattccgggtaccggagccaagtagagaacgagaaagggaatggttccaatttcattatcaacagattaagactcacagcggcggaatgggacagtgacaccacttactcctgtttagtgggtcacccgtccctcagccgggatttaatcagaagtataaataaatctcacggtaaaccgacattagttaatctttcagttgtactaagcgacactgttaaatcctgtacataatttgcagtgattgactaattgttttctatagataagttcatgttgttctggcaataacggtttaagcaaccgaaccaatgcgttttcaattcaacgcaaggcacagtcacatttctgatgagagaacacgtttgtaaaaataattagctactatttgaaattttatctgtcaatgaaggacaattatgattagaatctgatatgcaaggataacctgatcttgtcagtgcaatcgttctgaagatcccttgacgtgtttcacgccgttattgaaagaacagaaaatgatgctttagtgtgtatgtggcgccgatagttgcaataaacgcagaatgaaaactt

SEQ ID NO: 18 - DNA, 23 bp, Artificial Sequence
  Description of Artificial Sequence Synthetic primer
gcatgggtagaccaaacaccaag

SEQ ID NO: 19 - DNA, 22 bp, Artificial Sequence
  Description of Artificial Sequence Synthetic primer
gcgtcctcagagagagtcccta

SEQ ID NO: 20 - DNA, 24 bp, Artificial Sequence
  Description of Artificial Sequence Synthetic primer
gagacggacgaatcactgaccatc

SEQ ID NO: 21 - DNA, 25 bp, Artificial Sequence
  Description of Artificial Sequence Synthetic primer
gggtagaccaaacaccaagaacagc

SEQ ID NO: 22 - DNA, 24 bp, Artificial Sequence
  Description of Artificial Sequence Synthetic primer
gttctagccaataggaacgtatag

SEQ ID NO: 23 - DNA, 25 bp, Artificial Sequence
  Description of Artificial Sequence Synthetic primer
gtttgcacaagagagtagtctttac

SEQ ID NO: 24 - DNA, 24 bp, Artificial Sequence
  Description of Artificial Sequence Synthetic primer
cctaaattgtcacagcgaatcatg

SEQ ID NO: 25 - DNA, 22 bp, Artificial Sequence
  Description of Artificial Sequence Synthetic primer
gtgcagttccctagaagtcttg

SEQ ID NO: 26 - DNA, 23 bp, Artificial Sequence
  Description of Artificial Sequence Synthetic primer
gcatgggtagaccaaacaccaag

SEQ ID NO: 27 - DNA, 22 bp, Artificial Sequence
  Description of Artificial Sequence Synthetic primer
gcgtcctcagagagagtcccta

SEQ ID NO: 28 - DNA, 24 bp, Artificial Sequence
  Description of Artificial Sequence Synthetic primer
gagacggacgaatcactgaccatc

SEQ ID NO: 29 - DNA, 25 bp, Artificial Sequence
  Description of Artificial Sequence Synthetic primer
gggtagaccaaacaccaagaacagc

SEQ ID NO: 30 - DNA, 22 bp, Artificial Sequence
  Description of Artificial Sequence Synthetic primer
cacctcttccgacatgaggtcc

SEQ ID NO: 31 - DNA, 24 bp, Artificial Sequence
  Description of Artificial Sequence Synthetic primer
gattaaagaaaggaaaccaatgac

SEQ ID NO: 32 - DNA, 22 bp, Artificial Sequence
  Description of Artificial Sequence Synthetic primer
cagctacaaaagtaactcccac

SEQ ID NO: 33 - DNA, 25 bp, Artificial Sequence
  Description of Artificial Sequence Synthetic primer
gagagacaagaagtcaacgtctcat

SEQ ID NO: 34 - DNA, 23 bp, Artificial Sequence
  Description of Artificial Sequence Synthetic primer
gcatgggtagaccaaacaccaag

SEQ ID NO: 35 - DNA, 22 bp, Artificial Sequence
  Description of Artificial Sequence Synthetic primer
gcgtcctcagagagagtcccta

SEQ ID NO: 36 - DNA, 24 bp, Artificial Sequence
  Description of Artificial Sequence Synthetic primer
gagacggacgaatcactgaccatc

SEQ ID NO: 37 - DNA, 25 bp, Artificial Sequence
  Description of Artificial Sequence Synthetic primer
gggtagaccaaacaccaagaacagc

SEQ ID NO: 38 - DNA, 22 bp, Artificial Sequence
  Description of Artificial Sequence Synthetic primer
ggtgagacggtgagaaggtgcg

SEQ ID NO: 39 - DNA, 23 bp, Artificial Sequence
  Description of Artificial Sequence Synthetic primer
ggaagccaggaatggaaaggtag

SEQ ID NO: 40 - DNA, 22 bp, Artificial Sequence
  Description of Artificial Sequence Synthetic primer
ccaaaggtaggtaaaatcgacg

SEQ ID NO: 41 - DNA, 24 bp, Artificial Sequence
  Description of Artificial Sequence Synthetic primer
ggtctaaagaagttgactacctag

SEQ ID NO: 42 - DNA, 23 bp, Artificial Sequence
  Description of Artificial Sequence Synthetic primer
gcatgggtagaccaaacaccaag

SEQ ID NO: 43 - DNA, 22 bp, Artificial Sequence
  Description of Artificial Sequence Synthetic primer
gcgtcctcagagagagtcccta

SEQ ID NO: 44 - DNA, 23 bp, Artificial Sequence
  Description of Artificial Sequence Synthetic primer
gcatgggtagaccaaacaccaag

SEQ ID NO: 45 - DNA, 23 bp, Artificial Sequence
  Description of Artificial Sequence Synthetic primer
ccacctcttccgacatgaggtcc

SEQ ID NO: 46 - DNA, 23 bp, Artificial Sequence
  Description of Artificial Sequence Synthetic primer
ctcatttcatctgactactgacc

SEQ ID NO: 47 - DNA, 25 bp, Artificial Sequence
  Description of Artificial Sequence Synthetic primer
tcttccgaacatgaggtccaaagtg

SEQ ID NO: 48 - protein, 382 aa, Homo sapiens
MALQGISVVELSGLAPGPFCAMVLADFGARVVRVDRPGSRYDVSRLGRGKRSLVLDLKQPRGAAVLRRLCKRSDVLLEPFRRGVMEKLQLGPEILQRENPRLIYARLSGFGQSGSFCRLAGHDINYLALSGVLSKIGRSGENPYAPLNLLADFAGGGLMCALGIIMALFDRTRTGKGQVIDANMVEGTAYLSSFLWKTQKLSLWEAPRGQNMLDGGAPFYTTYRTADGEFMAVGAIEPQFYELLIKGLGLKSDELPNQMSMDDWPEMKKKFADVFAEKTKAEWCQIFDGTDACVTPVLTFEEVVHHDHNKERGSFITSEEQDVSPRPAPLLLNTPAIPSFKRDPFIGEHTEEILEEFGFSREEIYQLNSDKIIESNKVKASL

SEQ ID NO: 49 - protein, 220 aa, Homo sapiens
MTASSSSDYGQTSKMSPRVPQQDWLSQPPARVTIKMECNPSQVNGSRNSPDECSVAKGGKMVGSPDTVGMNYGSYMEEKHMPPPNMTTNERRVIVPADPTLWSTDHVRQWLEWAVKEYGLPDVNILLFQNIDGKELCKMTKDDFQRLTPSYNADILLSHLHYLRETPLPHLTSDDVDKALQNSPRLMHARNTGGAAFIFPNTSVYPEATQRITTRPVSYR

SEQ ID NO: 50 - protein, 758 aa, Homo sapiens [sequence omitted]

SEQ ID NO: 51 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 52 - protein, 79 aa, Homo sapiens
MDAEFRHDSGYEVHHQKLVFFAEDVGSNKGAIIGLMVGGVVIATVIVITLVMLKKKQYTSIHHGVVEVGKLDCMFPSGN

SEQ ID NO: 53 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 54 - protein, 265 aa, Homo sapiens
MGAAVFFGCTFVAFGPAFALFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSLLCRRQEDSRVMVYSALRIPPED

SEQ ID NO: 55 - protein, 501 aa, Homo sapiens
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 56 - protein, 418 aa, Homo sapiens [sequence omitted]

SEQ ID NO: 57 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 58 - protein, 146 aa, Homo sapiens
MAGPLRAPLLLLAILAVALAVSPAAGSSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

SEQ ID NO: 59 - protein, 617 aa, Rattus norvegicus
MKTFTLPASVLFCFLLLIRGLGAAPPGRSDVYPPPLGSEHNGQVAEDAVSRPKDDSVPEVRAARNSEPQDQGELFQGVDPRALAAVLLQALDRPASPPAVPAGSQQGTPEEAAEALLTESVRSQTHSLPASEIQASAVAPPRPQTQDNDPEADDRSEELEALASLLQELRDFSPSNAKRQQETAAAETETRTHTLTRVNLESPGPERVWRASWGEFQARVPERAPLPPSVPSQFQARMSENVPLPETHQFGEGVSSPKTHLGETLTPLSKAYQSLSAPFPKVRRLEGSFLGGSEAGERLLQQGLAQVEAGRRQAEATRQAAAQEERLADLASDLLLQYLLQGGARQRDLGGRGLQETQQERENEREEEAEQERRGGGEDEVGEEDEEAAEAEAEAEEAERARQNALLFAEEEDGEAGAEDKRSQEEAPGHRRKDAEGTEEGGEEDDDDEEMDPQTIDSLIELSTKLHLPADDVVSIIEEVEEKRKRKKNAPPEPVPPPRAAPAPTHVRSPQPPPPAPARDELPDWNEVLPPWDREEDEVFPPGPYHPFPNYIRPRTLQPPASSRRRHFHHALPPARHHPDLEAQARRAQEEADAEERRLQEQEELENYIEHVLLHRP

SEQ ID NO: 60 - protein, 317 aa, Homo sapiens [sequence omitted]

SEQ ID NO: 61 - protein, 233 aa, Homo sapiens
MPCRREEEEEAGEEAEGEEEEDDSFLLLQQSVTLGSSGEVDRLVAQIGETLQLDAAQDSPASPCAPPGVPLRAPGPLAAAVPADKARPPAVPLLLPPASAETVGPAPSGALRCALGDRGRVRGRAAPYCVAEVAAGPSALPGPCRRGWLRDAVTSRRLQQRRWTQAGARAGDDDPHRLLQQLVLSGNLIKEAVRRLQRAVAAVAATGPASAPGPGGGRSGPDRIALQPSGSLL

SEQ ID NO: 62 - protein, 757 aa, Homo sapiens
MLLRLLLAWAAAGPTLGQDPWAAEPRAACGPSSCYALFPRRRTFLEAWRACRELGGDLATPRTPEEAQRVDSLVGAGPASRLLWIGLQRQARQCQLQRPLRGFTWTTGDQDTAFTNWAQPASGGPCPAQRCVALEASGEHRWLEGSCTLAVDGYLCQFGFEGACPALQDEAGQAGPAVYTTPFHLVSTEFEWLPFGSVAAVQCQAGRGASLLCVKQPEGGVGWSRAGPLCLGTGCSPDNGGCEHECVEEVDGHVSCRCTEGFRLAADGRSCEDPCAQAPCEQQCEPGGPQGYSCHCRLGFRPAEDDPHRCVDTDECQIAGVCQQMCVNYVGGFECYCSEGHELEADGISCSPAGAMGAQASQDLGDELLDDGEDEEDEDEAWKAFNGGWTEMPGILWMEPTQPPDFALAYRPSFPEDREPQIPYPEPTWPPPLSAPRVPYHSSVLSVTRPVVVSATHPTLPSAHQPPVIPATHPALSRDHQIPVIAANYPDLPSAYQPGILSVSHSAQPPAHQPPMISTKYPELFPAHQSPMFPDTRVAGTQTTTHLPGIPPNHAPLVTTLGAQLPPQAPDALVLRTQATQLPIIPTAQPSLTTTSRSPVSPAHQISVPAATQPAALPTLLPSQSPTNQTSPISPTHPHSKAPQIPREDGPSPKLALWLPSPAPTAAPTALGEAGLAEHSQRDDRWLLVALLVPTCVFLVVLLALGIVYCTRCGPHAPNKRITDCYRWVIHAGSKSPTEPMPPRGSLTGVQTCRTSV

SEQ ID NO: 63 - protein, 190 aa, Homo sapiens
MATHHTLWMGLALLGVLGDLQAAPEAQVSVQPNFQQDKFLGRWFSAGLASNSSWLREKKAALSMCKSVVAPATDGGLNLTSTFLRKNQCETRTMLLQPAGSLGSYSYRSPHWGSTYSVSVVETDYDQYALLYSQGSKGPGEDFRMATLYSRTQTPRAELKEKFTAFCKAQGFTEDTIVFLPQTDKCMTEQ

SEQ ID NO: 64 - protein, 1426 aa, Homo sapiens
MLLRRRNGPCPFPLLLLLLAHCICIWPASAARDRYARQNNRQRHQDIDRDRDRDRFLYRSSSAQNRQRGGANFALGLGANGVTIPTSLEDKNKNEFVKGKICIGTKSRLSVPSNKEHHYRNLRDRYTNCTYVDGNLKLTWLPNENLDLSFLDNIREVTGYILISHVDVKKVVFPKLQIIRGRTLFSLSVEEEKYALFVTYSKMYTLEIPDLRDVLNGQVGFHNNYNLCHMRTIQWSEIVSNGTDAYYNYDFTAPERECPKCHESCTHGCWGEGPKNCQKFSKLTCSPQCAGGRCYGPKPRECCHLFCAGGCTGPTQKDCIACKNFFDEAVSKEECPPMRKYNPTTYVLETNPEGKYAYGATCVKECPGHLLRDNGACVRSCPQDKMDKGGECVPCNGPCPKTCPGVTVLHAGNIDSFRNCTVIDGNIRILDQTFSGFQDVYANYTMGPRYIPLDPERREVFSTVKEITGYLNIEGTHPQFRNLSYFRNLETIHGRQLMESMFAALAIVKSSLYSLEMRNLKQISSGSVVIQHNRDLCYVSNIRWPAIQKEPEQKVWVNENLRADLCEKNGTICSDQCNEDGCWGAGTDQCLTCKNFNFNGTCIADCGYISNAYKFDNRTCKICHPECRTCNGAGADHCQECVHVRDGQHCVSECPKNKYNDRGVCRECHATCDGCTGPKDTIGIGACTTCNLAIINNDATVKRCLLKDDKCPDGYFWEYVHPQEQGSLKPLAGRAVCRKCHPLCELCTNYGYHEQVCSKCTHYKRREQCETECPADHYTDEEQRECFQRHPECNGCTGPGADDCKSCRNFKLFDANETGPYVNSTMFNCTSKCPLEMRHVNYQYTAIGPYCAASPPRSSKITANLDVNMIFIITGAVLVPTICILCVVTYICRQKQKAKKETVKMTMALSGCEDSEPLRPSNIGANLCKLRIVKDAELRKGGVLGMGAFGRVYKGVWVPEGENVKIPVAIKELLKSTGAESSEEFLREAYIMASEEHVNLLKLLAVCMSSQMMLITQLMPLGCLLDYVRNNRDKIGSKALLNWSTQIAKGMSYLEEKRLVHRDLAARNVLVQTPSLVKITDFGLAKLLSSDSNEYKAAGGKMPIKWLALECIRNRVFTSKSDVWAFGVTIWELLTFGQRPHENIPAKDIPDLIEVGLKLEQPEICSLDIYCTLLSCWHLDAAMRPTFKQLTTVFAEFARDPGRYLAIPGDKFTRLPAYTSQDEKDLIRKLAPTTDGSEAIAKPDDYLQPKAAPGPSHRTDCTDEMPKLNRYCKDPSNKNSSTGDDERDSSAREVGVGNLRLDLPVDEDDYLMPTCQPGPNNNNNMNNPNQNNMAAVGVAAGYMDLIGVPVSVDNPEYLLNAQTLGVGESPIPTQTIGIPVMGGPGTMEVKVPMPGSEPTSSDHEYYNDTQRELQPLHRNRNTETRV

SEQ ID NO: 65 - protein, 334 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 66 - protein, 191 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRA

SEQ ID NO: 67 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 68 - protein, 208 aa, Homo sapiens [sequence omitted]

SEQ ID NO: 69 - protein, 436 aa, Homo sapiens
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIK

SEQ ID NO: 70 - protein, 426 aa, Homo sapiens
MFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 71 - protein, 233 aa, Homo sapiens [sequence omitted]

SEQ ID NO: 72 - protein, 33 aa, Human papillomavirus
TRSTNMSLCAAISTSETTYKNTNFKEYLRHGEE

SEQ ID NO: 73 - protein, 562 aa, Homo sapiens
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 74 - protein, 51 aa, Homo sapiens
MFLHISSPFKYPHTQEAQKEAQRSLGEMPGRHLGSSMSLALCLVPLVREGH

SEQ ID NO: 75 - protein, 177 aa, Homo sapiens
SVLGTTPSPVPTTSSISVLTTSTTSASTTSTTSGPGTTPSPVPTTSTTSAPTTSTTSAPTTSTTSAPTTSTPSAPTTSTTLAPTTSTTSAPTTSTTSTPTSSTSTTSAPTTSTTSTTSTPQTSTTSASTTSITCGPGTTPSPVPTTSTTSAPTTSTTSAATTSTISAPTTSTTSAPT

SEQ ID NO: 76 - protein, 345 aa, Homo sapiens
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 77 - protein, 1065 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 78 - protein, 102 aa, Mus musculus
MKLLAMVALLVTICSLEGALVKRQADGPDMQSLFTQYFQSMTDYGKDLMEKAKTSEIQSQAKAYFEKTHEQLTPLVRSAGTSLVNFFSSLMNLEEKPAPAAK

SEQ ID NO: 79 - protein, 600 aa, Rattus norvegicus
MKTFTLPASVLFCFLLLIRGLGAAPPGRSDVYPPPLGSEHNGQVAEDAVSRPKDDSVPEVRAARNSEPQDQGELFQGVDPRALAAVLLQALDRPASPPAVPAGSQQGTPEEAAEALLTESVRSQTHSLPASEIQASAVAPPRPQTQDNDPEADDRSEELEALASLLQELRDFSPSNAKRQQETAAAETETRTHTLTRVNLESPGPERVWRASWGEFQARVPERAPLPPSVPSQFQARMSENVPLPETHQFGEGVSSPKTHLGETLTPLSKAYQSLSAPFPKVRRLEGSFLGGSEAGERLLQQGLAQVEAGRRQAEATRQAAAQEERLADLASDLLLQYLLQGGARQRDLGGRGLQETQQERENEREEEAEQERRGGGEDEVGEEDEEAAEAEAEAEEAERARQNALLFAEEEDGEAGAEDKRSQEEAPGHRRKDAEGTEEGGEEDDDDEEMDPQTIDSLIELSTKLHLPADDVVSIIEEVEEKRKRKKNAPPEPVPPPRAAPAPTHVRSPQPPPPAPARDELPDWNEVLPPWDREEDEVFPPGPYHPFPNYIRPRTLQPPASSRRRHFHHALPPARHHPDLEAQARRAQEEADAEERRLQ

SEQ ID NO: 80 - protein, 180 aa, Human immunodeficiency virus 1
MENRWQVMIVWQVDRMRINTWKSLVKYHMHVSKKAKRWVYRHHYDSNHPRISSEVHIPLREAKLVVTTYWGLHTGEREWHLGQGVSIEWRMKSYRTQVDPSLADQLIHMHYFDCFSESAIRKAILGQIVSPRCDYQAGHNKVGSLQYLALTALINPKRTKPPLPSVKKLVEDRWNKPQKT

SEQ ID NO: 81 - protein, 530 aa, Homo sapiens
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQAATKQSNASSDVEVEEKETSVSKEDTDHEEKASNEDVTKAVDITTPKAARRGRKRKAEKQVETEEAGVVTTATASVNLKVSPKRGRPAATEVKIPKPRGRPKMVKQPCPSESDIITEEDKSKKKGQEEKQPKKQPKKDEEGQKEEDKPRKEPDKKEGKKEVESKRKNLAKTGVTSTSDSEEEGDDQEGEKKRKGGRNFQTAHRRNMLKGQHEKEAADRKRKQEEQMETEQQNKDEGKKPEVKKVEKKRETSMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSLAEQRQHEEANKTKDQGKKGPNKKLEKEQTGSKTLNGGSDAQDGNQPQHNGESNEDSKDNHEASTKKKPSSEERETEISLKDSTLDN

SEQ ID NO: 82 - protein, 342 aa, Caligus clemensi
MLSISIQNLITSVSYRNPRQTKTELLNVMKHYRSLHAKVEYFSFNDGVRKKLIVLKGTIPIKYKGSYYNIPLSFWLLDTHPANAPICYVNPTNSMSIKVSRNVDSSGKIYLPYLHEWNQNRSDLLSLIQICIITFSESSPVYAKSPFSGGEGSQPVDPVFSSLLEQQHPSSSNSTIPTLHLLNSVRSAVQDKLNVALREEFEIKGCELQSLRKMRDELNSGKVSIETAITESKRNTDALEKNVEDLEGKLKELEEANEKLEGEKEKDLDEALVGTAPLYNQLFHTFAKEASIDDAIYYLGEGLRRGVIDCDSFLKNVRKLSRQQFELRSLMIKCREKAGLRV

SEQ ID NO: 83 - protein, 138 aa, Human immunodeficiency virus 1
VPVWKDAETTLFCASDAKAHETEVHNVWATHACVPTDPNPQEIQLKNVTENFNMWKNNMVEQMQEDVISLWDQSLKPCVKLTPLCVTLNCTDATLTNSTYITNVSKIIGDITEEVRNCSFNMTTELRDKKQKVHALFL

SEQ ID NO: 84 - protein, 352 aa, Human immunodeficiency virus 1 [sequence omitted]

SEQ ID NO: 85 - protein, 1435 aa, Human immunodeficiency virus 1
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLREDLAFLQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 86 - protein, 671 aa, Homo sapiens
MAEFKEKPEAPTEQLDVACGQENLPVGAWPPGAAPAPFQYTPDHVVGPGADIDPTQITFPGCICVKTPCLPGTCSCLRHGENYDDNSCLRDIGSGGKYAEPVFECNVLCRCSDHCRNRVVQKGLQFHFQVFKTHKKGWGLRTLEFIPKGRFVCEYAGEVLGFSEVQRRIHLQTKSDSNYIIAIREHVYNGQVMETFVDPTYIGNIGRFLNHSCEPNLLMIPVRIDSMVPKLALFAAKDIVPEEELSYDYSGRYLNLTVSEDKERLDHGKLRKPCYCGAKSCTAFLPFDSSLYCPVEKSNISCGNEKEPSMCGSAPSVFPSCKRLTLETMKMMLDKKQIRAIFLFEFKMGRKAAETTRNINNAFGPGTANERTVQWWFKKFCKGDESLEDEERSGRPSEVDNDQLRAIIEADPLTTTREVAEELNVNHSTVVRHLKQIGKVKKLDKWVPHELTENQKNRRFEVSSSLILRNHNEPFLDRIVTCDEKWILYDNRRRSAQWLDQEEAPKHFPKPILHPKKVMVTIWWSAAGLIHYSFLNPGETITSEKYAQEIDEMNQKLQRLQLALVNRKGPILLHDNARPHVAQPTLQKLNELGYEVLPHPPYSPDLLPTNYHVFKHLNNFLQGKRFHNQQDAENAFQEFVESQSTDFYATGINQLISRWQKCVDCNGSYFD

SEQ ID NO: 87 - protein, 166 aa, Homo sapiens
HHHHMRVSFMVAMDENRVIGKDNNLPWRLPSELQYVKKTTMGHPLIMGRKNYEAIGRPLPGRRNIIVTRNEGYHVEGCEVAHSVEEVFELCKNEEEIFIFGGAQIYDLFLPYVDKLYITKIHHAFEGDTFFPEMDMTNWKEVFVEKGLTDEKNPYTYYYHVYEKQQ

SEQ ID NO: 88 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 89 - protein, 884 aa, Plasmodium falciparum
MEYFNCVNNLCCKFICTIRKYVKYFLYLKDSSYEIYNINLYNNNNMNIINNKNITNNKNITNNINNSFSNDYINYNHNYNHLNNSSSSKHNNYNVNNIDEKNIKNDYNTYHNIYEQIFFKYNPSFYEYLMFTLMKKLIHYKNYIFNKTKKINNSYNNNDIKNIDGFLIFQNINFEEIFLNTFYSSFPFKLFLHSLYMIFICFIYFVVLYFMLLKKIYTHPFIFHLSVLKFLFDIIFFLSFILYPLFLRLKRIDKIIYSSYISSYIFVCVTFLYSFIIFKCSSYSVKMNSNTYQNNFVFQNMLFLLINIIYICIFCFLKNYMILYSFLYNCRFSIFCILFIFLYYYLFFSLDFYRIIHLPLDNFFFPFLCFLFFSFLFIFKIIMSLYYEYVYEKKYRILFVKKNNLIEKRITKRKNTNINNAYFTKYFSIDNTIPTSPIEDILNNFKHILETINIIEENPNHNLTTNIMKIKEKIKNCDNILRTKNINQVQIGKYRKFEKVYNIWCLDKMYLNYPLNQEETKSFLSNSLNRISFNSFSNMHSLLSSKFQEHYNDIYDWNGNIENIYKANTFISIGYKLLYPLGVLEANFDKEKLKKFLFRICSYYNDIPYHTSLHAAQVAHFSKSMLFMLDMNHKISAIDEFCLHISSLCHDTGHPGLNNYFLINSENNLALTYNDNSVLENYHCSLLFKTLKNPNYNIFEHYPYHIFISCKKNIIKAILSTDMKNHFEYISDFRTSKEFIDYDNLSNDQIWQIFCLILKASDIGHSTLEWNKHLEWTLKINEEFYLQGLLEKSLNIQNSFLCDINTMNKLALSQIDFLKHLCIPLFNELNYICKNNDVYTHCIQPIENNIERWESHKNDNQNLGLHEKYKEENLLSKLELIKFE

SEQ ID NO: 90 - protein, 190 aa, Borrelia burgdorferi
EGAIKEVSELLDKLVKAVKTAEGASSGTAAIGEVVADADAAVRKDKASVKGIAKGIKEIVEAAGGSEKLKAVAAAKGENNKGAGKLFGKAGAAAHGDSEAASKAAGAVSAVSGEQILSAIVTAADAAEQDGKKPEEAKNPIAAAIGDKDGGAEFGQDEMKKDDQIAAAIALRGMAKDGKFAVKDGEKEKA

SEQ ID NO: 91 - protein, 805 aa, Homo sapiens [sequence omitted]

SEQ ID NO: 92 - protein, 150 aa, Homo sapiens
MYGKIIFVLLLSAIVSISASSTTGVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 93 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 94 - protein, 494 aa, Homo sapiens
MPRVYIGRLSYQARERDVERFFKGYGKILEVDLKNGYGFVEFDDLRDADDAVYELNGKDLCGERVIVEHARGPRRDGSYGSGRSGYGYRRSGRDKYGPPTRTEYRLIVENLSSRCSWQDLKDYMRQAGEVTYADAHKGRKNEGVIEFVSYSDMKRALEKLDGTEVNGRKIRLVEDKPGSRRRRSYSRSRSHSRSRSRSRHSRKSRSRSGSSKSSHSKSRSRSRSGSRSRSKSRSRSQSRSRSKKEKSRSPSKEKSRSRSHSAGKSRSKSKDQAEEKIQNNDNVGKPKSRSPSRHKSKSKSRSRSQERRVEEEKRGSVSRGRSQEKSLRQSRSRSRSKGGSRSRSRSRSKSKDKRKGRKRSREESRSRSRSRSKSERSRKRGSKRDSKAGSSKKKKKEDTDRSQSRSPSRSVSKEREHAKSESSQREGRGESENAGTNQETRSRSRSNSKSKPNLPSESRSRSKSASKTRSRSKSRSRSASRSPSRSRSRSHSRS

SEQ ID NO: 95 - protein, 623 aa, Homo sapiens
MPPKVTSELLRQLRQAMRNSEYVTEPIQAYIIPSGDAHQSEYIAPCDCRRAFVSGFDGSAGTAIITEEHAAMWTDGRYFLQAAKQMDSNWTLMKMGLKDTPTQEDWLVSVLPEGSRVGVDPLIIPTDYWKKMAKVLRSAGHHLIPVKENLVDKIWTDRPERPCKPLLTLGLDYTGISWKDKVADLRLKMAERNVMWFVVTALDEIAWLFNLRGSDVEHNPVFFSYAIIGLETIMLFIDGDRIDAPSVKEHLLLDLGLEAEYRIQVHPYKSILSELKALCADLSPREKVWVSDKASYAVSETIPKDHRCCMPYTPICIAKAVKNSAESEGMRRAHIKDAVALCELFNWLEKEVPKGGVTEISAADKAEEFRRQQADFVDLSFPTISSTGPNGAIIHYAPVPETNRTLSLDEVYLIDSGAQYKDGTTDVTRTMHFGTPTAYEKECFTYVLKGHIAVSAAVFPTGTKGHLLDSFARSALWDSGLDYLHGTGHGVGSFLNVHEGPCGISYKTFSDEPLEAGMIVTDEPGYYEDGAFGIRIENVVLVVPVKTKYNFNNRGSLTFEPLTLVPIQTKMIDVDSLTDKECDWLNNYHLTCRDVIGKELQKQGRQEALEWLIRETQPISKQH

SEQ ID NO: 96 - protein, 906 aa, Homo sapiens
MCRIAGALRTLLPLLAALLQASVEASGEIALCKTGFPEDVYSAVLSKDVHEGQPLLNVKFSNCNGKRKVQYESSEPADFKVDEDGMVYAVRSFPLSSEHAKFLIYAQDKETQEKWQVAVKLSLKPTLTEESVKESAEVEEIVFPRQFSKHSGHLQRQKRDWVISPINLPENSRGPFPQELVRIRSDRDKNLSLRYSVTGPGADQPPTGIFIINPISGQLSVTKPLDREQIARFHLRAHAVDINGNQVENPIDIVINVIDMNDNRPEFLHQVWNGTVPEGSKPGTYVMTVTAIDADDPNALNGMLRYRIVSQAPSTPSPNMFTINNETGDIITVAAGLDREKVQQYTLIIQATDMEGNPTYGLSNTATAVITVTDVNDNPPEFTAMTFYGEVPENRVDIIVANLTVTDKDQPHTPAWNAVYRISGGDPTGRFAIQTDQNSNDGLVTVVKPIDFEANRMFVLTVAAENQVPLAKGIQHPPQSTATMSVTVIDVNENPYFAPNPKIIRQEEGLHAGTMLTTFTAQDPDRYMQQNIRYTKLSDPANWLKIDPVNGQITTIAVLDRESPNVKNNIYNATFLASDNGIPPMSGTGTLQIYLLDINDNAPQVLPQEAETCETPDPNSINITALDYGIDPNAGPFAFDLPLSPVTIKRNWTITRLNGDFAQLNLKIKFLEAGIYEVPIIITDSGNPPKSNISILRVKVCQCDSNGDCTDVDRIVGAGLGTGAIIAILLCIIILLILVLMFVVWMKRRDKERQAKQLLIDPEDDVRDNILKYDEEGGGEEDQDYDLSQLQQPDTVEPDAIKPVGIRRMDERPIHAEPQYPVRSAAPHPGDIGDFINEGLKAADNDPTAPPYDSLLVFDYEGSGSTAGSLSSLNSSSSGGEQDYDYLNDWGPRFKKLADMYGGGDD

SEQ ID NO: 97 - protein, 362 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 98 - DNA, 21 bp, Artificial Sequence
  Description of Artificial Sequence Synthetic primer
ggtccagctgctcgagtctgg

SEQ ID NO: 99 - DNA, 26 bp, Artificial Sequence
  Description of Artificial Sequence Synthetic primer
tagtgcaccaccatcaccatcactaa

SEQ ID NO: 100 - DNA, 22 bp, Artificial Sequence
  Description of Artificial Sequence Synthetic primer
tgcctaacggcgtgtcattatg

SEQ ID NO: 101 - DNA, 10 bp, Artificial Sequence
  Description of Artificial Sequence Synthetic oligonucleotide
catgccatgg

SEQ ID NO: 102 - DNA, 10 bp, Artificial Sequence
  Description of Artificial Sequence Synthetic oligonucleotide
ccatggnnnn

SEQ ID NO: 103 - protein, 116 aa, Mus sp.
QVQLQESGAEVMKPGASVKISCKATGYTFSTYWIEWVKQRPGHGLEWIGEILPGSGSTYYNEKFKGKATFTADTSSNTAYMQLSSLTSEDSAVYYCARGDGNYGYWGQGTTVTVSS

SEQ ID NO: 104 - protein, 133 aa, Camelus sp.
DVQLQASGGGSVQAGGSLRLSCAASGYTIGPYCMGWFRQAPGKEREGVAAINMGGGITYYADSVKGRFTISQDNAKNTVYLLMNSLEPEDTAIYYCAADSTIYASYYECGHGLSTGGYGYDSWGQGTQVTVSS

SEQ ID NO: 105 - protein, 125 aa, Lama glama
QVQLQESGGGLVQPGGALRLSCAASGLTLADTATGWFRQAPGKEREWVACISRRKAGTFYADSLKDRFTISRDDSVNTVYLQIISLKPDDTANYVCVQIPFCNEFMRGDLDRAILGQGTQVTVAS

SEQ ID NO: 106 - protein, 112 aa, Unknown
  Description of Unknown Shark V-NAR polypeptide
RVDQTPRSVTKETGESLTINCVLRDASYALGSTCWYRKKSGEGNEESISKGGRYVETVNSGSKSFSLRINDLTVEDGGTYRCGLGVAGGYCDYALCSSRYAECGDGTAVTVN